SEQUENCE LISTING (71 sequences)

SEQ ID NO: 1 - DNA, 20 bp, Artificial sequence
  Synthetic PCR amplification primer
agagtttgatcmtggctcag

SEQ ID NO: 2 - DNA, 19 bp, Artificial sequence
  Synthetic PCR amplification primer
tccgtgccagcagccgccg

SEQ ID NO: 3 - DNA, 20 bp, Artificial sequence
  Synthetic PCR amplification primer
cggytaccttgttacgactt

SEQ ID NO: 4 - DNA, 22 bp, Artificial sequence
  Synthetic PCR amplification primer
gacgttgtaaaacgacggccag

SEQ ID NO: 5 - DNA, 21 bp, Artificial sequence
  Synthetic PCR amplification primer
gaggaaacagctatgaccatg

SEQ ID NO: 6 - protein, 11 aa, Artificial sequence
  Target (no.914) peptide available from Sigma-Aldrich-Fluka
RPKPQQFFGLM

SEQ ID NO: 7 - DNA, 49 bp, Artificial sequence
  Target (no. 1256) DNA resolvase substrate available from Sigma Genosys
gacgctgccgaattctggcttgctaggacatctttgcccacgttgaccc

SEQ ID NO: 8 - DNA, 35 bp, Artificial sequence
  Target (no. 1261) DNA substrate available from sigma Genosys
taagctccggattgtccgggaggtaaagccctgat

SEQ ID NO: 9 - DNA, 25 bp, Artificial sequence
  Target (no. 1262) DNA substrate available from sigma Genosys
cacaggaagctctacaggtactccg

SEQ ID NO: 10 - DNA, 70 bp, Artificial sequence
  Target (no. 1263) DNA substrate available from Sigma Genosys
tggtcatcagggctttacctcccggacaatccggagcttacggagtacctgtagagcttcctgtgcaagc

SEQ ID NO: 11 - DNA, 20 bp, Artificial sequence
  Target (no. 1265) DNA Helicase substrate dsDNA available from Sigma Genosys
gcactggccgtcgttttacc

SEQ ID NO: 12 - protein, 4 aa, Artificial sequence
  Target (no. 1921) peptide substrate available from Sigma-Aldrich-Fluka
AAPF

SEQ ID NO: 13 - DNA, 405 bp, Unknown
  Isolated from unknown organism from coastal seawater of Kolguev Island, Barents sea, Russia (KOL)
atgtggatacctcgcaacaaggctaagtcgagtgccagtaaaggcaaaaccggtgatagcagtcatcgtgccgcaggcgaacgccgagaattagacgccgaagagtggctgatacaacgtggctttgtacccataacccgcaattaccgcacgcgcggcggcgaaatcgatctgattatgcgcgacgccgatacccttgtgtttgtagaagtacgttatcgtaaaaccacggagcacggcacgggggcagaaaccattacctatcacaaacagcagcgactacgtcgtgctgccctacactacctgcaaaagcattttggtagccgcgaaccgccttgtcgatttgatgttatgtcaggtactggcgacccagttatcttcgattggattagtaatgcgttttaa

SEQ ID NO: 14 - protein, 134 aa, Unknown
  Synthetic Construct
MWIPRNKAKSSASKGKTGDSSHRAAGERRELDAEEWLIQRGFVPITRNYRTRGGEIDLIMRDADTLVFVEVRYRKTTEHGTGAETITYHKQQRLRRAALHYLQKHFGSREPPCRFDVMSGTGDPVIFDWISNAF

SEQ ID NO: 15 - protein, 8 aa, Unknown
  Isolated from unknown organism from coastal seawater of Kolguev Island, Barents Sea, Russia (KOL)
TTEHGTGA

SEQ ID NO: 16 - protein, 8 aa, Unknown
  Isolated from unknown organism from coastal seawater of Kolguev Island, Barents Sea, Russia (KOL)
PPCRFDVM

SEQ ID NO: 17 - DNA, 23 bp, Artificial sequence
  Synthetic PCR amplification primer
acnacngargarggnacnggngc

SEQ ID NO: 18 - DNA, 24 bp, Artificial sequence
  Synthetic PCR amplification primer
catnacntcnaanckrcanggngg

SEQ ID NO: 19 - DNA, 30 bp, Artificial sequence
  Synthetic PCR amplification primer
atgtggatacctcgcaacaaggctaagtcg

SEQ ID NO: 20 - DNA, 36 bp, Artificial sequence
  Synthetic PCR amplification primer
ttaaaacgcattactaatccaatcgaagataactgg

SEQ ID NO: 21 - DNA, 519 bp, Unknown
  Isolated from unknown organism from coastal seawater of Kolguev Island, Barents Sea, Russia (KOL)
atgcatcagttggcagtcaccggcaagcaggcagctgatgtggctgtgctgctgggcggtcaacagcttgaggtgcaccggatagagcgggatgacgtcttgatccagcacctgatcgagctggaacgtcagttctggcattacgttgaaaccgacacaccgccaccagccgacggctctgattccgctgacatggcactgcgtctgctctacccggaagacacaggtatggttattgacctctctcaggatcagtccctgaacgagtcctataccgagctcaagcaggtacggcagtcactctctgatctgagcacccgagaatctgttctcaagcagcgtcttcaagagtccatggggtcagccagtaaagccgtgtttgccaatggctctatcacttggaagaaagccaaggatggcatcgccatggatatggaggccctgttcaaggcgcaccctgacctgaaaacccaataccagatcagcaagcctggcagcaggcgctttctggtcaattaa

SEQ ID NO: 22 - protein, 172 aa, Unknown
  Synthetic Construct
MHQLAVTGKQAADVAVLLGGQQLEVHRIERDDVLIQHLIELERQFWHYVETDTPPPADGSDSADMALRLLYPEDTGMVIDLSQDQSLNESYTELKQVRQSLSDLSTRESVLKQRLQESMGSASKAVFANGSITWKKAKDGIAMDMEALFKAHPDLKTQYQISKPGSRRFLVN

SEQ ID NO: 23 - protein, 11 aa, Unknown
  Isolated from unknown organism from coastal seawater of Kolguev Island, Barents Sea, Russia (KOL)
GVPKYVEVQVM

SEQ ID NO: 24 - protein, 6 aa, Unknown
  Isolated from unknown organism from coastal seawater of Kolguev Island, Barents Sea, Russia (KOL)
ASKAVF

SEQ ID NO: 25 - DNA, 33 bp, Artificial sequence
  Synthetic PCR amplification primer
ggngtnccnaantangtngargtncargtnatg

SEQ ID NO: 26 - DNA, 18 bp, Artificial sequence
  Synthetic PCR amplification primer
raanacngcnttnswngc

SEQ ID NO: 27 - DNA, 24 bp, Artificial sequence
  Synthetic PCR amplification primer
atgcatcagttggcagtcaccggc

SEQ ID NO: 28 - DNA, 27 bp, Artificial sequence
  Synthetic PCR amplification primer
ttaattgaccagaaagcgcctgctgcc

SEQ ID NO: 29 - DNA, 888 bp, Unknown
  Isolated from unknown organism from coastal seawater of Kolguev Island, Barents Sea, Russia (KOL)
attgtaatacgactcactatagggcgaattgggcccgacgtcgcatgctcccggccgccatggcggccgcgggaattcgattatgtggccagctatcgcttcggccatcagatttacacccccgatgacatcgaaatagcgtcattgatagaagacgatcgcccttatgcgggcctagcctatgctggcctgtcgatcttccaatcccgggaccaggggcaatggcgcgatagtcgtgcctggcacatggatctgggcctagtcggcccgggtgccggaggccagcgcttccagagtgctgtccatggtgctaccggcagtgatgagcccaaaggttgggacaatcagcttgagaatgaacccttcttcaacgtggcctacgggcagcgttggtggcggcagtcccgcctgggatctttggagcttgaatacgggcctgcaatgggcgcggcggctggcaatctttacacctatgcttccagcggccttggcttgcgctttggcaaagggctggaacacagcttggggttaccgtctatcaacccgggctatggtagcggcgcctacttcgagccggggcaatcgttcgcctggttcggttacgtcaatgttgatggccgttacatggctcataacatgctgctggacggaaacaccttcagcaacagccattccgtagaccgggagcaatgggtcggcgatctgcaggcgggtatcgccctgacctgggagcgctggcaagtgagctttgccagtgtgtggcgcacccgggagttcaagggccagactgaacccgatcaatttgggtcgctggtggaatcactagtgaattcgcggccgcctgcaggtcgaccatatgggagagctcccaacgcgttggatgcatga

SEQ ID NO: 30 - protein, 295 aa, Unknown
  Synthetic Construct
IVIRLTIGRIGPDVACSRPPWRPREFDYVASYRFGHQIYTPDDIEIASLIEDDRPYAGLAYAGLSIFQSRDQGQWRDSRAWHMDLGLVGPGAGGQRFQSAVHGATGSDEPKGWDNQLENEPFFNVAYGQRWWRQSRLGSLELEYGPAMGAAAGNLYTYASSGLGLRFGKGLEHSLGLPSINPGYGSGAYFEPGQSFAWFGYVNVDGRYMAHNMLLDGNTFSNSHSVDREQWVGDLQAGIALTWERWQVSFASVWRTREFKGQTEPDQFGSLVESLVNSRPPAGRPYGRAPNALDA

SEQ ID NO: 31 - protein, 9 aa, Unknown
  Isolated from unknown organism from coastal seawater of Kolguev Island, Barents sea, Russia (KOL)
PYAGLAYAG

SEQ ID NO: 32 - protein, 10 aa, Unknown
  Isolated from unknown organism from coastal seawater of Kolguev Island, Barents sea, Russia (KOL)
WVGDLQAGIA

SEQ ID NO: 33 - DNA, 26 bp, Artificial sequence
  Synthetic PCR amplification primer
ccntangcnggnytngcntaygcngg

SEQ ID NO: 34 - DNA, 26 bp, Artificial sequence
  Synthetic PCR amplification primer
aanganganganmgnccntaygcngg

SEQ ID NO: 35 - DNA, 28 bp, Artificial sequence
  Synthetic PCR amplification primer
gcnatnccngcytgnarytcnccnaccc

SEQ ID NO: 36 - DNA, 24 bp, Artificial sequence
  Synthetic PCR amplification primer
atgtggccagctatcgcttcggcc

SEQ ID NO: 37 - DNA, 24 bp, Artificial sequence
  Synthetic PCR amplification primer
tcaaaacgcccgagccaccaccag

SEQ ID NO: 38 - DNA, 474 bp, Unknown
  Isolated from unknown organism from acidic of Porto Levante, Vulcanic Island, Italy (VUL)
atgttgcgcttggtgcaggggggattcagtaatatggcagcaccgattcagaaagtcatccgacaggaacccgtcaaaaatccgcttgagtctgggagcccgtcggcttcggaggaacttcaactattggttgaagaactgcatcagagcggggttctcgaggctgcacggtcgatgcttggagcaaaggattccatcgccaaaatcctggtcgagcaactgctgagaaaggatgtactgacgcttatcaacaatttgatggcggcaggcaccgttctgacaaagctcgatccagcgcagctcgagcgcttgaccgaaggactgagtgccggggtaacagaggcacatcagacaatcgaagcgaatcagtcaatcagtatcatgggacttttgaagacattgcaagaccccgatgtaaaccgggcactccagtttgccatcgggttcctcaggggtctcgggaaaactatctga

SEQ ID NO: 39 - protein, 157 aa, Unknown
  Synthetic Construct
MLRLVQGGFSNMAAPIQKVIRQEPVKNPLESGSPSASEELQLLVEELHQSGVLEAARSMLGAKDSIAKILVEQLLRKDVLTLINNLMAAGTVLTKLDPAQLERLTEGLSAGVTEAHQTIEANQSISIMGLLKTLQDPDVNRALQFAIGFLRGLGKTI

SEQ ID NO: 40 - protein, 8 aa, Unknown
  Isolated from unknown organism from acidic of Porto Levante, Vulcanic Island, Italy (VUL)
VEELHQSG

SEQ ID NO: 41 - protein, 6 aa, Unknown
  Isolated from unknown organism from acidic of Porto Levante, Vulcanic Island, Italy (VUL)
EAARSM

SEQ ID NO: 42 - protein, 6 aa, Unknown
  Isolated from unknown organism from acidic of Porto Levante, Vulcanic Island, Italy (VUL)
PDVNRA

SEQ ID NO: 43 - protein, 6 aa, Unknown
  Isolated from unknown organism from acidic of Porto Levante, Vulcanic Island, Italy (VUL)
QFAIGF

SEQ ID NO: 44 - DNA, 23 bp, Artificial sequence
  Synthetic PCR amplification primer
gtnganganntncancarwsngg

SEQ ID NO: 45 - DNA, 18 bp, Artificial sequence
  Synthetic PCR amplification primer
gangcngcnmgnwgnatg

SEQ ID NO: 46 - DNA, 16 bp, Artificial sequence
  Synthetic PCR amplification primer
cncknttnacrtcngg

SEQ ID NO: 47 - DNA, 18 bp, Artificial sequence
  Synthetic amplification primer
raanccnatngcraaytg

SEQ ID NO: 48 - DNA, 21 bp, Artificial sequence
  Synthetic PCR amplification primer
atgttgcgcttggtgcagggg

SEQ ID NO: 49 - DNA, 21 bp, Artificial sequence
  Synthetic PCR amplification primer
ctagatagttttcccgagacc

SEQ ID NO: 50 - DNA, 486 bp, Unknown
  Isolated from unknown organism from brine-seawater interface above hypersaline anoxic basin L′Atalante, Eastern Mediterranean Sea (L′A)
ttattgatggcagagatgacagaagaaaaattagctgagtatttagaacctttatcagaaattttatcacgctatgaaaaaaaagagagatatttaattccggttttacaggaagctcaggaggaatatggttatttaccggaagaagtaatgaaagaaatagcattgggcttaaatctttctttaagtcaggtatatggggttgtaacattttacagtcagtttcatcaggagccaagaggtaataatattattcgggtttgtctgggaacagcctgtcatgttagaggtggagatggaatcttaaatgctattaaagatgaactgggaattgatgcaggagaaacaactgatgatttagaatttacacttgaatctgtggcctgtattggtgcctgtggtctggctccagttataatggtcaatgatgatacccacggccgtttaactccggaaaaagttcctgaaattatggcaaagtataaa

SEQ ID NO: 51 - protein, 162 aa, Unknown
  Synthetic Construct
LLMAEMTEEKLAEYLEPLSEILSRYEKKERYLIPVLQEAQEEYGYLPEEVMKEIALGLNLSLSQVYGVVTFYSQFHQEPRGNNIIRVCLGTACHVRGGDGILNAIKDELGIDAGETTDDLEFTLESVACIGACGLAPVIMVNDDTHGRLTPEKVPEIMAKYK

SEQ ID NO: 52 - protein, 11 aa, Unknown
  Isolated from unknown organism from brine-seawater interface above hypersaline anoxic basin L′Atalante, Eastern Mediterranean Sea (L′A)
LLMAEMTEEKL

SEQ ID NO: 53 - protein, 14 aa, Unknown
  Isolated from unknown organism from brine-seawater interface above hypersaline anoxic basin L′Atalante, Eastern Mediterranean Sea (L′A)
KEIALGLNLSLSQV

SEQ ID NO: 54 - protein, 12 aa, Unknown
  Isolated from unknown organism from brine-seawater interface above hypersaline anoxic basin L′Atalante, Eastern Mediterranean Sea (L′A)
PEKVPEIMAKYK

SEQ ID NO: 55 - DNA, 40 bp, Artificial sequence
  Synthetic PCR amplification primer
gacgacgacaagatgatgttattgatggcagagatgacag

SEQ ID NO: 56 - DNA, 44 bp, Artificial sequence
  Synthetic PCR amplification primer
gaggagaagcccggttatttatactttgccataatttcaggaac

SEQ ID NO: 57 - DNA, 5210 bp, Unknown
  Multifunctunal alpha/beta-hydrolase library of a microbial community in petroleum hydrocarbons
cgctcccgcacgaatatgaacgctgtgaaattccggtgtatatgcgccaacaaggaccaaatcatgccagtcaagccaaactatggcctaataaggtctgggtttacgagtaccgcctaagtactattggccatcgtcgaatcctaacaggtgactacacatctgtttatccacccatcgataacaataaggtttactgatgaatctcgttaaaagcctgataatactgcttgtgatcgcagtgatcggaatatttgctagcgtccactttgctccgttcgaaacagcttcttgtcttgtcgatattaaacgcaatatcgccggcctagagcgcaaaagcatttccctcgccgatggtaaccaatatgtttatcttgaaggcggcaagggtgaaaccttggtattacttcacggttttggtgccgataaagataactttaccgaagttagcccttacttaacgggcgacttccatgttattgctcccgaccacattggctttggtgaatcctctaagccaacaggcgcagattatagccctatcgctcaagcgcagcgtttgcatgaattggttgcgcgcctcggtttagagcgcttccacttaggtggaagctcaatgggtggccacatagcgatgacatacgccacgctatacccgcatgaagttaagagcttatggttactcgacccaggtggtgtttggtccgctccggaagccgaaatgcgcactatcattcgtaaaactggggtgaatccgttaacagctaaaaccccagaagagttccgtaaagtattcgatatcgtgatgagcaagccccctttcatcccaggctttgtactcgatgaaatggcgaaaaagcgtatcgctaacttcgatttagaacagaatatttttgcccaactatctgcagacaatgtcgaagaacgcgtccgcggcctaaccacccctaccttgctagtatggggcgctgaagatcgagtactcaacccagaagcagcacccattctggaaggacttttgaccaatgttaaaacgattatcatgccaggtattggtcacctgcctatgctagaagcgccaaagcaaacagcaaccgacttaaaagcatttattgctgatttgcctgaataaaactaataggcgttgaatttaacagcgcccagaaaaaagcggccctctgcaaataaacaggggccgctttttttatacttaaaaggtcggattaattagcttcaatgctcacatctaaagacatttcaccaccgttctcatccgtaccggacgccgaaaaagccagttgatcatcactcgttgcggtaatttcaaacgcataagtcgccgtgctctgacaggcatcatcactctcatcacacgtaccttcgccataaatatcactttgaccggaaataatatcctgcgagaaggtgttgtcgtaaaccaaagttacgttcaatccgattgtgctcgcatcgccagaaaacgctgccttctcccccgccagcgtcatgatttcagcgtaattgacagcggatatagaggtgttgctattcaccacaccggcaccaactcccttcgaagcaccaacacacgctaacgaatcacttaattctgcaaaggtggcactggcggaacccacagtaaaatcgacacctatcgcgtattcactagcggcatcggtgaacgaagtggcatcgctaaccttcacacccaccatgcgcgtattttgcgcaatgctgtacccgtcccccgtcgcatcagtctgcaagctgccagacaagctgcggttgttgggttactatcagactccaattgtaagagtcctcaaccgagccacttaatgtgccatcagcaagcgttaacgtgatatcggtaaaatctaattcgtgagcaatattacatgcccaaaagcggtactcaccttcggtttctaatgcgctcagtaataccagttggcgaccttgatagtagttggtggtatcactgctatcggtcacttcataatcaaacttcgaactggtatttatcagccataagccttcaggggtatctgaacttaatgccgcatccgctgctggaagatctgcccccaattcagctaaggtttgttgcgcaagttgcgtggtagggtcaacgtcgatcacggtagcgacattggtactgctactgttaccactgcaggccgataccagtgcagcaacgaccaagcaataggccgactttttgaattgggagtgcatcatgatttccttttgatattcttatttaatcctagaaggggatcatagcagacgcaattgcagacctaagggcaattcgggcccattaccaatacgattatccccacttaccttggcctcctagcgttcacgccgttataatcgcccgcagaaattcatcctcctatcctcacgggactatctcatgacctctaaaaccgtcgtttccgaacgcctgtatgatggctacgcccaatccttcactgtgagcgaactgctgtttgaagtgaaaacggaacaccaacatctggaaatcttcgaaacgccgtttctaggccgcgttatgctgctggatggagtggtacaaaccaccgagaaagacgaatttatttaccacgaaagcatggtccatgtgcctttgtttgcccacccagcccctaacgtgtgctaattattggtggcggcgacggcggcatcttgcgtgaagtgttgcgccacaaaaacgtagaacacgtaacccaagttgaaatcgacggcagcgtcatcgacatgtgcaaagaatacttcccacgtcattctaatggtgccttcgatgacccgcgcgccactatcgtgatcgccgatggcaaagaattcgtcgccaactgccaagacaaatacgacgtcatcatctccgactccaccgaccctatcggcccaggcgaagtgctgtttacctccgatttctatgccgacgaaaaaacctgcctgaacgaaggcggcatcatggtggcacagaacggcgtgccgtttatgcaaggccaagaaatcaccaataccttccagcgcctaagcaaactgtacgcggacaacagcttctacgttgcccccgtgccaacctatgcaggtggttttatgaccttagcctgggcaaccgacgacgcctcattgcgcaagcaaagcgttgaacagattcaagcgcgttacgacgccgcagggtttagcacacgttattacaacccagagattcatgttgcggcatttgcattgccgaattatgtgaaagctttgatggtgtgattttatgcttgaagtcaaatgncttcgaccaatcttgtaggagggagtcagcgaacgttttttgttcgtactcccgattactgcagttttcgtcactgctcgctgtcgctccctgagtgactcctacagaaagatccacccttggttttaagtccgtgttgcgaaaagaactcgcggcgtcagccgcaaaacccaccaatagctacaaaatcccaccgtcactgttataatctcgcgtctttaaattttcgaacgttgcataacgcacgcaaacaggaatcctcgctgtgagccagagtcgccataacgtcaaaacattccaaggcttaatcgctgccttgcaggaatactggtccgaacagggctgtgtaatcaaccaaccactcgatatggaagtcggtgccggtactttccataccgcgacgtttttgcgtgctattggcccagaaaactggagtgctgcttatgttcaaccaagccgccgtcctactgatggccgctatggtgaaaacccgaaccgcttgcaacattactaccaatttcaggtagtgatgaagccaaacccagtggatattcaagaaaagtaccttgagtcgctgcgcgtgatgggcgttgatcctttggttcacgatattcgtttcgttgaagacaactgggaatcaccaacgctaggcgcttggggtttgggctgggaagtttggcttaacggtatggaagtgactcagttcacttacttccagcaagttggcggtttggaatgtttccccgtaaccggcgaaatcacttacggtcttgagcgtatcgccatgtacctgcaagaagtggattctgtctacgacttagtttggacttacggcccagacggcaaagctgtgacctacggcgatgtgttccatcaaaacgaagtagaacagtcggcctataacttcgaacacgccgatgtcgatttcttattcaaagccttcgaccaatacgagaaagactgcaaacgtctgatcgaagttggcttgccgctacccgcttacgagcaggtattgaaaggctcgcatacctttaacttactcgatgctcgcggcgcgatttctgtgactgagcgtcagggctatatcttgcgtgtacgtaccttagcgcgctcggttgctgaagcttacttcaacagccgtgccgaaaaaggcttcccgctggcgaccgaagcaaaccgcgccgaagtattagcgaagtacgaagcagccaaggcgaaaaaagccgataaagacgctgcccagcaggagaccaaataatgagcactcgtgatttcttagtagagctaggcaccgaagagctgccaccgaaagcgcttaaaaatctgtctaacgcgtttgcccagggcattgaacaaggtttgaaagacgccggtttaaccatgggtgcgattgaacaattcgccgcgccacgtcgtttagccgtgcgcatttcggagttaccagagcagcaagccgatcaagaagaagtgctatacggcccgccagccaacatcgcctttgacgccgatggtaagccaaccaaagccgccttaggctttgcggcccgcgccggtgccgatgcgtctgaattaaaaacagcgccagattctgacaaaaagaatgccggtaagctaatgctcgaacgtacgatcaaaggcaaaaataccactgagctattggccgctattgtgcaaaacagcttagataagttgccgattcctaagcgtatgcgctggggttcgtcacgtattgaattcgtgcgccccgtacagtggttagtcatgctgtttggtaacgacgtggtcgatgccgaagcgcttggcttaaaagccggcaacaccagccgtggtcaccgcttccatgcgccgggcgagatccaaagaattcaaaaagcttctcgagagtacttctagagcggccgcgggcccatcg

SEQ ID NO: 58 - DNA, 933 bp, Unknown
  Multifunctunal alpha/beta-hydrolase mined from a metagenome library of a microbial community in seawater contaminated with petroleum hydrocarbons
atgaatctcgttaaaagcctgataatactgcttgtgatcgcagtgatcggaatatttgctagcgtccactttgctccgttcgaaacagcttcttgtcttgtcgatattaaacgcaatatcgccggcctagagcgcaaaagcatttccctcgccgatggtaaccaatatgtttatcttgaaggcggcaagggtgaaaccttggtattacttcacggttttggtgccgataaagataactttaccgaagttagcccttacttaacgggcgacttccatgttattgctcccgaccacattggctttggtgaatcctctaagccaacaggcgcagattatagccctatcgctcaagcgcagcgtttgcatgaattggttgcgcgcctcggtttagagcgcttccacttaggtggaagctcaatgggtggccacatagcgatgacatacgccacgctatacccgcatgaagttaagagcttatggttactcgacccaggtggtgtttggtccgctccggaagccgaaatgcgcactatcattcgtaaaactggggtgaatccgttaacagctaaaaccccagaagagttccgtaaagtattcgatatcgtgatgagcaagccccctttcatcccaggctttgtactcgatgaaatggcgaaaaagcgtatcgctaacttcgatttagaacagaatatttttgcccaactatctgcagacaatgtcgaagaacgcgtccgcggcctaaccacccctaccttgctagtatggggcgctgaagatcgagtactcaacccagaagcagcacccattctggaaggacttttgaccaatgttaaaacgattatcatgccaggtattggtcacctgcctatgctagaagcgccaaagcaaacagcaaccgacttaaaagcatttattgctgatttgcctgaataa

SEQ ID NO: 59 - protein, 310 aa, Unknown
  Synthetic Construct
MNLVKSLIILLVIAVIGIFASVHFAPFETASCLVDIKRNIAGLERKSISLADGNQYVYLEGGKGETLVLLHGFGADKDNFTEVSPYLTGDFHVIAPDHIGFGESSKPTGADYSPIAQAQRLHELVARLGLERFHLGGSSMGGHIAMTYATLYPHEVKSLWLLDPGGVWSAPEAEMRTIIRKTGVNPLTAKTPEEFRKVFDIVMSKPPFIPGFVLDEMAKKRIANFDLEQNIFAQLSADNVEERVRGLTTPTLLVWGAEDRVLNPEAAPILEGLLTNVKTIIMPGIGHLPMLEAPKQTATDLKAFIADLPE

SEQ ID NO: 60 - DNA, 36 bp, Artificial sequence
  Synthetic PCR amplification primer
gacgacgacaagatggcaacctgcggcgaagtactg

SEQ ID NO: 61 - DNA, 35 bp, Artificial sequence
  Synthetic PCR amplification primer
gaggagaagcccggtcaacccaacaccgcggcctg

SEQ ID NO: 62 - DNA, 1633 bp, Pseudomonas putida
atggcaacctgcggcgaagtactggtcaaactccttgaaggctatggcgtcgatcatgtcttcggcatccccggtgtgcataccgtggagctctatcgtggcctggcgggctcttccattcgccacatcaccccgcgtcatgagcaaggtgccgggttcatggctgacggctatgcgcgcacccgcggcaaacccggggtatgcttcatcatcactggcccgggcatgaccaatatcaccactgccatgggccaggcctatgccgactcgatcccgatgctggtgatttccagcgtgcagtcccgtgaccaactgggcggtggccgtggcaagttgcacgagctgcccaaccaagccgcgctggtatcgggggtggcggcgttttcccacaccctgatgagtgcagccgacttgccgcaggtgctggcccgggcatttgccgtgttcgacagtgcccggccacgcccggtgcatatcgaaatcccgctcgatgtgctggttgaaccggccgacttcctgctgccgggccgccctgtacgtggcagtcgggctggggctgcaccgcaggccgtggcgcagatggctgagcgactggccagtgcacgtcggccgctgattctggccggtggcggggcgttggctgcgggcgctgcgctggcacgcctggccgaacaccttcaggccccggtggcgctgaccatcaatgccaaaggcttgctgccagccagccacccattgcagatcggctcgacccagtcgttacccgccacacgggcgctggtggccgaggccgacgtggtgcttcggtaccgaactggctgaaaccgattatgacgtgaccttcaagggaggcttcgagatcccgggcaggctgctgcgtattgacatcgacccggaccagaccgtgcgcaattatctgccggagctggcgctggtagccgatgccgagctggcagccgaagcgctgctcggtgccgtgcaagcccagccgcagccagtgcacgaaagcacttggggcgtggcccgcgtggccgacctgcgcaaggtcctggcagcagactgggaccagccaaccctgagccagacacgtttgctgagcgccatactggagcgcctgcctgatgcgattctggtgggcgactcgacccaacctgtgtacaccggcaacctgacactcgacatgcagcagccacgccgctggttcaacgcctcgaccggttacggcaccttgggttacgcgttgccagcagccatgggcgcctggttgggcagtgccgagcaagccgtcgaacgcgctccggcggtgtgcctgattggtgatggtggtttgcagttcaccctgccggaactggccagtgcagtggaggcgcaggtaccgctgatcgtactgctgtggaataaccaggggtacgaggaaatcaagaaatacatggtcaatcgggcgatcgagccagtcggggtggatatccataccccggatttcatcggcgtggcgcgggcgttgggcgcggcagcagagaacgtggccgatatcgcccaattgcaggccgcgctcgggcaagcggtggagcgcaaggggccgaccttgattcaggtggaccagaatcagtggcaggccgcggtgttgggttga

SEQ ID NO: 63 - DNA, 39 bp, Artificial sequence
  Synthetic PCR amplification primer
gacgacgacaagatgttcgaatctgccgaaatcggccac

SEQ ID NO: 64 - DNA, 40 bp, Artificial sequence
  Synthetic PCR amplification primer
gaggagaagcccggctatttcttgtgcttggcaaacgccg

SEQ ID NO: 65 - DNA, 1494 bp, Pseudomonas putida
atgttcgaatctgccgaaatcggccacagcatcgacaaggaggcttacgacgccgaggtacccgctttacgcgaggccctgctcgaagcccagtacgaactcaagcagcaggcgcgttttccggtgatcgtgctgatcaacggcattgaaggcgccggcaagggtgagacggtaaaactgctcaacgagtggatggacccgcgcatgatcgatgtgctcaccttcgaccagcagaccgacgaagagctggcccgcccgcccgcctggcgctattggcgggccttgccgcccaaggggcgaatgggcgttttctttggcaactggtacagccagatgctgcaggggcgggtgcacggggtgttcaaggatgccgtgctcgatcaggccattaccggtgccgaacgcctggaggagatgctgtgcgatgaaggtgcgctgatcatcaagttctggttccacctgtccaagaagcagatgaaggcacggctgaaatcgctcaaggacgacccgctgcacagctggaagatcagcccgctggattggcagcagtcgcaaacctacgaccgtttcgtgcgctttggcgagcgcgtgctgcgccgcaccagccgtgactatgcgccatggcacatcatcgaaggggtagacccgaattaccgcagcctggcggtggggcgcattctgctggaaagcctgcaagccgcgctggcccacaatcccaagggcaagcaccagggcaacgtggctccattgggccgcagcatcgacgaccgcagcctgcttggcgccctggacatgaccttgcgcctggacaaggccgactatcaggaacagttgatcaccgaacaggcgcgtctggccggcctgctgcgtgacaagcgcatgcgccggcacgccctggtggcggtgttcgaaggcaacgacgccgccggcaaggggggcgccatccgccgcgtggcggcggcgctggacccgcgtcagtaccgtatcgtaccgattgccgcgccgaccgaagaagagcgcgcccagccgtacctgtggcggttctggcggcacatcccggcacgcggcaagttcaccatcttcgaccgttcctggtatggccgggtgctggtggagcgggtggaaggcttctgcagccccgctgactggatgcgtgcctatagcgagatcaacgactttgaagagcagttggtggatgccggcgtggtggtggtcaagttctggctggcgatcgaccagcagacccaactggagcgcttcgaagagcgtgagcagattccgttcaaacgctacaagatcaccgaggatgactggcgcaaccgcgacaagtgggacgaatactcccaggcggtgggcgacatggtcgaccgcaccagcagcgagattgccccttggacgctggtggaggccaatgacaagcgctgggcgcgggtgaaggtggtgcgcacaatcaaccaggcgcttgaggcggcgtttgccaagcacaagaaatag

SEQ ID NO: 66 - DNA, 39 bp, Artificial sequence
  Synthetic PCR amplification primer
gacgacgacaagatggaaaaatcagcaaccgggtggatc

SEQ ID NO: 67 - DNA, 41 bp, Artificial sequence
  Synthetic PCR amplification primer
gaggagaagcccggttaggcatatttcctggccccgtaaac

SEQ ID NO: 68 - DNA, 867 bp, Pseudomonas putida
atggaaaaatcagcaaccgggtggatcaacggtttcataggcgtcgccatttttgcgggttcgttgccggcaacccgagtggcagtggccgacttcgaaccgacgttcctcacctgtgcccgggcaacaattgccgccatgctgggcgcactttttctgatcgtgctacgccagcctcgacccaaacggcgggatttgtcgcctttggctgtaactgcgctcggcgttgttatcggtttcccgctactgacagcatttgcccttcagcacataagctctgctcactccattgtttttgtcgggctcctgccattgtgtaccgcaggatttgcggttctgcggggcggtgaacgacctcggccattgttctggctgttctcgttgacaggtgccgggttggtcgctggctatgcgttgatgaatggaggcgaggcgtcggcggtgggcgatctgctgatgttggctgcggttgtggtctgtgggttgggctatgccgaaggagcgcgcctctcgcggacattgggtggttggcaggtgatcagctgggcgttgctggtagcgttgccgttcatgctgctgctgacggtggtcaatcttccagcccctgatgactttgccagggtaagtgcccctgcgtggttcagctttggctacgtttcactgttcagcatgctgatcgggtttgtgttctggtaccgagggctcgtccagggggggattgcggcagtgggccaattgcaactctttcagccgttcatggggcttgggctggcagcgttgcttctgcacgagcatgtcagctggacgatgctgctcgtgacgctgggtgctgtcatctgtgtttacggggccaggaaatatgcctaa

SEQ ID NO: 69 - DNA, 39 bp, Artificial sequence
  Synthetic PCR amplification primer
gacgacgacaagatgcgtgattaccagtggttgcatgag

SEQ ID NO: 70 - DNA, 36 bp, Artificial sequence
  Synthetic PCR amplification primer
gaggagaagcccggtcaatggttgacggtgtgcgcc

SEQ ID NO: 71 - DNA, 672 bp, Pseudomonas putida
atgcgtgattaccagtggttgcatgagtattgcctgaaccgctttggttcggcccaggcgctggaggctttcctgccgcagccgcgcacgccggcgcaactgcgcgacatcagtgacgaccgctacctgtcgacattggccctgcgcgtgttccgcgcggggctcaagcacagcctggtggatgccaagtggccggcgttcgagcaggtgttctttggcttcgacccggagaaagtggtgctgatgggcgccgagcatctggagcggttgatgcaggatgagcgcattatccgccacctgggcaagctcaagagcgtgccacgcaatgcgcaaatggtgctggatatcgccaaggcgcatggcagttttggtgcattcatcgccgattggccagtgaccgacattgtcggcttgtggaagtacctggccaagcacggcaaccagttgggcgggttgtcggcgccacggttcttgcgcatggtcggcaaggacacgttcatcccgaccgatgacatggcagcggcgttgattgcgcagaaggtgatcgacaagcagccaaccagccagcgcgacctggctctggtgcagcaggcgttcaaccagtggcatgcagagagcgggcggccgctgtgccagttgtcggtgatgctggcgcacaccgtcaaccattga

(19) United States
(12) Patent Application Publication
(10) Pub. No.: US 2012/0231972 A1
(43) Pub. Date: Sep. 13, 2012

(54) Probe Compound for Detecting and Isolating Enzymes and Means and Methods Using the Same

(76) Inventors: Peter N. Golyshin, Wolfenbuettel (DE); Olga V. Golyshina, Wolfenbuettel (DE); Kenneth N. Timmis, Wolfenbuettel (DE); Tatyana Chernikova, Braunschweig (DE); Agnes Waliczek, Braunschweig (DE); Manuel Ferrer, Madrid (ES); Ana Beloqul, Madrid (ES); Maria E. Guazzaroni, Madrid (ES); Jose M. Vieltes, Madrid (ES); Florencio Pazos, Madrid (ES); Antonio Lopez De Lacey, Madrid (ES); Victor M. Fernandez, Madrid (ES)

(21) Appl. No.: 13/257,383

(22) PCT Filed: Mar. 19, 2010
(86) PCT No.: PCT/EP10/01770

§ 371 Date: Apr. 27, 2012

Related U.S. Application Data
(60) Provisional application No. 61/210,482, filed on Mar. 19, 2009.

(30) Foreign Application Priority Data
Mar. 19, 2009 (EP) EP 09 003 977.7

(51) Int. Cl. C40B 30/08; C40B 50/14; G01N 21/64; C07F 15/06; C07D 403/14; C40B 40/04; C12N 9/96; B82Y 15/00; B82Y 5/00

(52) U.S. Cl. 506/11; 506/15; 506/30; 435/4; 435/188; 562/594; 558/169; 548/312.1; 977/914; 977/920

(57) ABSTRACT

The present invention relates to a probe compound that can comprise any substrate or metabolite of an enzymatic reaction in addition to an indicator component, such as, for example, a fluorescence dye, or the like. Moreover, the present invention relates to means for detecting enzymes in form of an array, which comprises any number of probe compounds of the invention which each comprise a different metabolite of interconnected metabolites representing the central pathways in all forms of life. Moreover, the present invention relates to a method for detecting enzymes involving the application of cell extracts or the like to the array of the invention which leads to reproducible enzymatic reactions with the substrates. These specific enzymatic reactions trigger the indicator (e.g. a fluorescence signal) and bind the enzymes to the respective cognate substrates. Moreover, the invention relates to means for isolating enzymes in form of nanoparticles coated with the probe compound of the invention. The immobilisation of the cognate substrates or metabolites on the surface of nanoparticles by means of the probe compounds allows capturing and isolating the respective enzyme, e.g. for subsequent sequencing.

### BACKGROUND OF THE INVENTION

The present invention relates to a probe compound for detecting and isolating enzymes, to a method for producing the probe compound, to means for detecting enzymes and means for isolating enzymes, to a method for producing the means for detecting and isolating enzymes, and to a method for detecting and isolating enzymes using the same.

In more detail, the present invention relates to a probe compound that can comprise any substrate or metabolite of an enzymatic reaction in addition to an indicator component, such as, for example, a fluorescence dye, or the like. Moreover, the present invention relates to means for detecting enzymes in form of an array. The array according to the present invention comprises any number of probe compounds of the invention which each comprise a different metabolite of interconnected metabolites representing the central pathways in all forms of life. The probe compound and the array of the invention can be used for detecting specific enzyme-substrate interactions associated with the corresponding substrate(s) or metabolite(s), which allows to identify substrate-specific enzymatic activity in a sample. Moreover, the present invention relates to a method for detecting enzymes involving the application of cell extracts or the like to the array of the invention which leads to reproducible enzymatic reactions with the substrates. These specific enzymatic reactions trigger the indicator (e.g. a fluorescence signal) and bind the enzymes to the respective cognate substrates. Moreover, the invention relates to means for isolating enzymes in form of nanoparticles coated with the probe compound of the invention. The immobilisation of the cognate substrates or metabolites on the surface of nanoparticles by means of the probe compounds allows capturing and isolating the respective enzyme, e.g. for subsequent sequencing. In short, the probe compound of the invention provides two new aspects: first, that only an active protein (enzyme) triggers the indicator signal, and second, that the active protein subsequently binds to the probe compound.

In recent years, the new discipline of “functional genomics” has greatly accelerated the research on the genomic basis of life processes in health and disease, and has significantly improved our understanding of such processes, their regulation and underlying mechanisms. However, until relatively recently, functional genomics has been sequence-centric, that is, functional assignments and metabolic network reconstructions have mostly depended on the genome sequence of the organism in question, combined with bioinformatic analyses based on homologies to known gene/protein-relationships. In addition to the fact that a significant fraction of genes in databases available today has a questionable annotation, many are not annotated at all, which adds further uncertainties to analyses and predictions based on them. With the recent advent of “metabolomics”, functional insights into the metabolic state of a cell became possible independently of sequence information. However, problems of metabolite identification and quantification still exist, and the link with the cognate metabolic pathways is still heavily dependent upon sequence-based metabolic reconstructions. Furthermore, it is currently very difficult to derive global metabolic overviews of non-sequenced organisms or communities of organisms existing in an individual habitat or biotop (biocoenosis).

Therefore, there exists an urgent need to solve the twin problems of the identification of metabolites and the enzymes involved in their transformation and, simultaneously, to begin the critical process of rigorous annotation of yet un-annotated and incorrectly annotated open reading frames (genes), and thereby improving the utility of the exponentially growing body of genome sequence information. Thus, an activity-based, annotation-independent procedure for the global assessment of cellular responses is urgently needed.

“Microarrays” or “biochips” have proven to be an important and indispensable tool for the fast gain and processing of information required in the field. Here, the term ‘array’ refers to a collection of a large number of different test compounds preferably arranged in a planar plane, e.g. by attachment on a flat surface, such as a glass slide surface, or by occupying special compartments or wells provided on a plate, such as a micro-titre plate. The test compounds, which are also referred to as probes, probe compounds or probe molecules, are usually bound or immobilised on the flat surface or to the walls of a compartment, respectively. The use of arrays allows for the rapid, simultaneous testing of all probe molecules with respect to their interaction with an analyte or a mixture of analytes in a sample. The analytes of the sample are often referred to as target molecules. The advantage of a planar array over a test (assay) having immobilised probe molecules on mobile elements, such as, for example, beads, is that in an array the chemical structure and/or the identity of the immobilised probe molecules is precisely defined by their location in the array surface. A specific local test signal, which is produced, for example, by an interaction between the probe molecule and the analyte molecule, can accordingly be immediately assigned to a type of molecule or to a probe molecule. As evidence of an interaction between a probe molecule and an analyte molecule, it is also possible to use the enzymatic conversion of the probe by the biomolecule, with the result that a local test signal can also disappear and accordingly serves as direct evidence. Particularly in miniaturised form, arrays having biological probe molecules are also known as “biochips”.

Usually, the surface of the microarray having the bound probe molecules is brought into contact, over its entire area, with the solution of the analyte molecules from a sample. Then, the solution is usually removed after a predetermined incubation time. Alternatively, appropriate amounts of the sample solution are filled into the respective compartments (wells) of the array. When the specific and selective interaction between the probe molecule and an analyte molecule is complete, a signal is generated at the location of the probe molecule. That signal can either be produced directly, for example by binding of a fluoresence-labelled biomolecule, or can be generated in further treatments with detection reagents, for example in the form of an optical or radioactive signal. Many different technical details relating to procedure and detection are well known and completely described in the art. There are numerous array protocols and processes which are adapted for automatic handling by corresponding (robotic) apparatuses, thus allowing for high reliability and reproducibility of information gain and processing.

Examples of known arrays in the prior art are nucleic acid arrays of DNA fragments, cDNAs, RNAs, PCR products, plasmids, bacteriophages and synthetic PNA oligomers, which are selected by means of hybridisation, with formation of a double-strand molecule, to give complementary nucleic acid analytes. In addition, protein arrays of antibodies, proteins expressed in cells or phage fusion proteins (phage display) play an important part. Furthermore, compound arrays of synthetic peptides, analogues thereof, such as peptoids, oligocarbamates or generally organic chemical compounds, are known, which are selected, for example, by means of binding to affinitive protein analytes or other analytes by means of enzymatic reaction. Moreover, arrays of chimaeras and conjugates of the said probe molecules have been described.

DNA microarray technology has a vast potential for improving the understanding of microbial systems. Microarray-based genomic technology is a powerful tool for viewing the expression of thousands of genes simultaneously in a single experiment. While this technology was initially designed for transcriptional profiling of a single species, its applications have been dramatically extended to environmental applications in recent years. The use of microarrays to profile metagenomic libraries may also offer an effective approach for characterizing many clones rapidly. As an example, a fosmid library was obtained and further arrayed on a glass slide. This format is referred to as a metagenome microarray (MGA). In the MGA format, the ‘probe’ and ‘target’ concept is a reversal of those of general cDNA and oligonucleotide microarrays: targets (fosmid clones) are spotted on a slide and a specific gene probe is labelled and used for hybridization. This format of microarray may offer an effective metagenome-screening approach for identifying clones from metagenome libraries rapidly without the need of laborious procedures for screening various target genes.

However, one of the greatest challenges in using microarrays for analyzing environmental samples is the low detection sensitivity of microarray-based hybridization in combination with the low biomass often present in samples from environmental settings. Microarrays for expression profiling can be divided into two broad categories, microarrays based on the deposition of preassembled DNA probes (cDNA microarrays) and those based on in situ synthesis of oligonucleotide probes (e.g. Affymetrix arrays, oligonucleotide microarrays). Applications employing DNA microarrays include, for example, the characterization of microbial communities from environmental samples such as soil and water. Various types of DNA microarrays have been applied to study the microbial diversity of various environments. Those include, for example, oligonucleotides, cDNA (PCR amplified DNA fragments), and whole genome DNA.

One of the major problems associated with nucleic acid-based micro-arrays is derived from the short half-life of mRNA, and that mRNA in bacteria and archaea usually comprise only a small fraction of total RNA. Moreover, the study of the gene expression from an environmental sample using DNA microarrays is a challenging task. First, the sensitivity may often be a part of the problem in PCR-based cDNA microarrays, since only genes from populations contributing to more than 5% of the community DNA can be detected. Second, samples often contain a variety of environmental contaminants that affects the quality of RNA and DNA hybridization and makes it difficult to extract undegraded mRNA. The specificity of the extraction method plays a central role and should vary depending on the site of sampling, as there must be sufficient discrimination between probes. However, there is a promising perspective for microarrays in determining the relative abundance of a microorganism bearing a specific functional gene in a complex environment.

However, specificity is a key issue, since one needs to distinguish the differences in hybridization signals due to population abundance from those due to sequence divergence. Furthermore, annotation and the comprehensive functional characterization of proteins or RNA molecules remain difficult, error-prone processes, but systems microbiology relies heavily on a thorough understanding of the functions of gene products.

At the moment, after DNA micro-arrays, the peptide arrays are the most popular. In this kind of arrays, peptides with different chemical composition are synthesised and immobilized on glass slides. The peptides may also contain a marker, such as a fluorescence dye marker (e.g. a fluorescent cyanine dye known under the name ‘Cy3’), but here the detection method is only based on the lowered fluorescence obtained with a protein bound to the molecule. There is no enzymatic reaction necessary for the signal, so un-specific bindings may occur and trigger a signal, which may lead to incorrect assignments. Further, there is no possibility to reconstruct metabolic networks.

Another array alternative is to bind proteins to a slide. Such system is usually not based on the detection of a fluorescence signal, but rather on the utilization of surface Plasmon resonance. This system has been exploited for the analysis of molecular interactions, i.e. protein-protein or molecule-protein interactions.

In view of the problems encountered in the prior art, the present invention is therefore based on the object of providing a novel probe compound, which allows for the testing of a reactive interaction of an enzyme with a small molecule or enzymatic substrate. The probe compound should allow for the easy linkage of all small molecules or substrates necessary for the life functions of an organism or communities living in a habitat (biocoenosis). Thus, a plurality of probe compounds should allow for the construction of a ‘reactome array’ or microarray which allows for the testing of all life supporting enzymatic reactions of an organism or community simultaneously. Particularly, the probe compound should provide a highly sensitive, accurate, reproducible, and robust high-throughput tool for a genome-wide analysis of the metabolic status of an organism or community. In this context, the term “genome-wide analysis” means an analysis that is independent of genome sequence. Moreover, the probe compound should also allow for use in the isolation of an enzyme so that said enzyme may be further analysed or identified in a subsequent step. Moreover, the probe compound should also allow for the identification of small molecules, substrates and/or metabolites which are metabolised by an organism or community, thus allowing the identification of biologic pathways or the direct comparison of the reactomes of different organisms, which might be applied in the search for new targets for drug-screening.

### SUMMARY OF THE INVENTION

The object of the invention is solved by a probe compound comprising a transition metal complex and a reactive component comprising a test component and an indicator component, wherein the test component and the indicator component are linked to form the reactive component, and wherein the reactive component is linked to the transition metal complex. The probe compound of the invention provides a means for testing of a reactive interaction of an enzyme with a small molecule or enzymatic substrate. The probe compound may be readily used in combination with all small molecules or substrates necessary for the life functions of an organism or communities living in a habitat (biocoenosis). Further, the probe compound provides a highly sensitive, accurate, reproducible, and robust high-throughput tool for a genome-wide analysis of the metabolic status of an organism or community. It allows the fast and reliable detection of a substrate specific enzyme interaction. Moreover, the probe compound may be readily used to detect the involvement of one enzymatic substrate in different metabolic pathways. Moreover, the probe compound provides a means to immobilise a substrate-specific enzyme, which can be advantageously used to isolate this enzyme from a sample.

Preferably, the object of the invention is solved by a probe compound for detecting specific enzyme-substrate interactions comprising a transition metal complex and a reactive component of general formula (X):

His-L_{His-TC}-TC-L_{TC-IC}-IC-L_{IC-His}-His formula (X)

wherein His represents a histidine residue, TC represents a test component, IC represents an indicator component, and each of L_{His-TC}, L_{TC-IC} and L_{IC-His} independently represents optional linker components,
wherein the reactive component is linked to the transition metal complex by the two histidine residues.

A preferred embodiment of the probe compound of the invention can be illustrated by the following general formula (1):

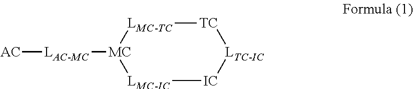

wherein AC represents an optional anchoring component, MC represents the transition metal complex, TC represents the test component, IC represents the indicator component, and L_{AC-MC}, L_{MC-TC}, L_{TC-IC} and L_{MC-IC} each independently represents an optional linker component between the respective components indicated by the subscripts, wherein it is preferred that L_{MC-TC} and L_{MC-IC} each independently comprise a histidine residue.

In more detail, the present invention relates to a probe compound that can comprise any substrate or metabolite of an enzymatic reaction in addition to an indicator component, such as, for example, a fluorescence dye, or the like. In short, the probe compound of the invention provides two new aspects: first, that only an active protein (enzyme) triggers the indicator signal, and second, that the active protein subsequently binds to the probe compound.

The object of the invention is also solved by a method for preparing the probe compound of the invention. The inventive method provides a versatile method for preparing all embodiments of the probe compound. Moreover, the method of the invention can be used for the identical and reproducible production of probe compounds comprising different enzymatic substrates, which allows for the ready use in automatic processes, such as parallel synthesis or the like.

The object is also solved by an array which comprises a plurality of different probe compounds of the invention. The array (which is sometimes referred to as “reactome array” in the following) can be used for the simultaneous detection of all reactive interactions between the probe compounds and analyte molecules (enzymes) from a sample. The array also provides a fast and reliable way to detect all metabolic pathways active in an organism or community, and may be used advantageously for an activity-based, annotation-independent procedure for the global assessment of cellular responses. The array can include a number of interconnected metabolites representing central pathways in all forms of life. The application of cell extracts to the array leads to reproducible enzymatic reactions with substrates that trigger the indicator signal and bind enzymes to cognate substrates.

The invention also provides a method for producing an array according to the invention, which allows for a versatile, fast and reproducible production of arrays according to the invention.

Moreover, the object is also solved by an isolation means comprising a probe compound according to the invention and a nanoparticle, preferably a magnetic nanoparticle. The isolation means according to the invention allows for the substrate specific interaction and binding of an enzyme which can then be isolated by means, such as, for example, filtration, gravitation force (centrifugation), an external magnetic force, or the like. The invention also provides a method for producing an isolation means according to the invention. The immobilisation of the cognate substrates or metabolites on the surface of nanoparticles by means of the probe compounds allows capturing and isolating the respective enzyme, e.g. for subsequent sequencing.

Moreover, the object is solved by a method for detecting enzymes using the probe compound according to the invention, or the array according to the invention, as well as by a method for isolating enzymes, using the isolation means according to the invention.

The particular subject-matter of the invention and its preferred embodiments will be described in more detail in the following description as well as in the examples and figures attached thereto.

### BRIEF DESCRIPTION OF THE FIGURES

FIG. 1 shows a schematic overview of the array strategy, including a summary of the four major steps in the construction and analysis of arrays. Steps 1 and 2: extensive data and synthetic mining effort to produce a library of metabolites than can be arrayed on glass slides in a spatially-addressable manner; steps 3 and 4: detection and analysis of enzymatic reactions following application of cell lysates to the array, and metabolic reconstructions.

FIG. 2 shows an overview of the array strategy, including a summary of the four major steps in the construction of metabolite complexes. (1) 2-step synthesis of bi-functional, non-fluorescent Cy3 dye component; (2) preparation of the His-tagged substrate (1-indanone is shown as a model substrate), (3) preparation of Co²⁺ linker molecules; and (4) synthesis of Cy3-metabolite complexes containing an amine with a nitrogen-to-metabolite ‘labile’ bond proximal to the catalysis reaction site.

FIG. 3 illustrates the reactome strategy. The generic structure of reactome metabolites involves three linked components, the enzyme substrate-metabolite, the quenched dye, and the linker used to immobilize the complex on the array or on nanoparticles. The substrate-metabolite is linked to the quenched dye though a labile nitrogen bond, and both the dye and the substrate are anchored to the Co(II)-containing poly (A) linker by histidine ‘tags’. Details of the synthetic strategy are provided in FIGS. 2 and 8.

An enzyme-catalysed chemical change in the substrate at a position adjacent to the weakly amine region causes rupture of the labile nitrogen:metabolite bond, and release of the quenched Cy3 dye. This in turn provokes release of the reaction product and the histidine ‘tags’ anchored to the Co(II), thereby exposing an active cobalt cation which ligates and immobilizes the enzyme on the array spot. The released dye is no longer quenched and gives a fluorescent signal. The nature of the reaction and the catalysis product is defined by the position to which the quenched dye and the substrate are linked (see table 2).

FIG. 4 shows Dose-response curves determined with pure E. coli β-Gal and Cy3-linked X-Gal. (A) Substrate dose response with fixed amount of β-Gal (5 ng/ml); (B) β-Gal dose response with fixed amount of Cy3-modified X-Gal (2.52 pmol/ml). For each experiment, normalized intensity values and fit curves were scaled relative to the maximum asymptotic values of the fit.

FIG. 5 shows the Receiver Operating Characteristic (ROC) curve of the array. The ROC shows the capacity of the array to discriminate compounds potentially metabolised by P. putida from those which are not metabolised. The “true positive rate” (TRP) is plotted on the Y-axis against the “false positive rate” (FRP) on the X-axis. The diagonal line represents the discriminative power of a random method.

FIG. 6 shows an overall comparison of metabolites transformed by lysates of the three communities KOL, VUL and L'A. (A) Pairwise comparisons of the compounds metabolized by lysates of the KOL, VUL and L'A metagenome libraries. (B) Overall comparison of compounds metabolized by the three libraries. (C) Pairwise comparisons of the compounds metabolized by lysates of the individual metagenome libraries and that of P. putida.

FIG. 7 shows dose-response curves determined with purified P. putida KT2440 proteins (A) and metagenomic proteins (B). Left and right figures represent protein and molecule dose responses. Results shown are the average of three independent assays, and were corrected for background signal. Results are not fitted to any model. The spotting process was carried out using a MicroGrid II micro-arrayer (Biorobotics) by spotting 0.25 nL droplets of SMs-Cy3 solutions (spot size 400 μm diameter with concentrations ranging from 0 to 0.25 pmol/ml) and further arrayed with 60 μl of a solution of pure enzyme (from 16 to 90 ng/ml in PBS buffer, depending on the enzyme used) (left column) or by spotting 0.25 nL droplets of SMs-Cy3 solution (spot size 400 μm diameter with concentration of 0.4 pmol/ml) and further arrayed with 60 μl of solution of pure enzyme at different concentrations (from 0 to 6000 μg/ml in PBS buffer). Signals were analyzed and quantified using GenePix pro 4.1 software (Axon). As shown, Cy3 fluorescence emission increased with increasing the amount of both protein and substrate, whereas inactive proteins did not (see below). (C) FTIR spectrum of L'A62 hydrogenase. Inset shows the H₂-uptake activity using methyl viologen as acceptor.

FIG. 8 summarizes the major steps used for the construction of metabolite complexes corresponding to the 26 different synthetic methods. Abbreviations used are as follows: 1,8-BDN (1,8-bis-(dimethylamino)-naphthalene); REBr (hybrid halogenase/dehalogenase; MeOH (methanol), (E) compound (Cy3 intermediate containing histidine and linkers).

FIG. 9 shows representative molecules of the different synthetic methods used to link metabolites with histidine and the dye molecule.

### DETAILED DESCRIPTION OF THE INVENTION

The present invention provides a probe compound comprising a transition metal complex and a reactive component comprising a test component and an indicator component, wherein the test component and the indicator component are linked to form the reactive component, and wherein the reactive component is linked to the transition metal complex. The probe compound of the invention thus comprises the following components: a transition metal complex and a reactive component. The reactive component in turn comprises the following components: a test component and an indicator component, which are linked to form the reactive component. Further, the reactive component is linked to the transition metal complex.

A preferred embodiment of the probe compound of the invention can be illustrated by the following general formula (1):

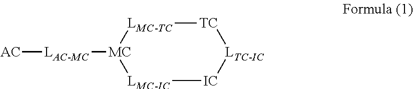

wherein AC represents an optional anchoring component, MC represents the transition metal complex, TC represents the test component, IC represents the indicator component, and L_{AC-MC}, L_{MC-TC}, L_{TC-IC} and L_{MC-IC} each independently represents an optional linker moiety between the respective components indicated by the subscripts.

Preferably, the probe compound for detecting specific enzyme-substrate interactions comprises a transition metal complex and a reactive component of general formula (X):

His-L_{His-TC}-TC-L_{TC-IC}-IC-L_{IC-His}-His formula (X)

wherein His represents a histidine residue, TC represents a test component, IC represents an indicator component, and each of L_{His-TC}, L_{TC-IC} and L_{IC-His} independently represents optional linker components,
wherein the reactive component is linked to the transition metal complex by the two histidine residues.

Herein, the term “linked” means that two components are connected with each other by means of at least one chemical bond, preferably by one, two, or three chemical bonds, and most preferred by exactly one chemical bond. A chemical bond indicates any chemical bond known in the art, such as, for example, an ionic bond, a covalent bond, including a single bond, a double bond, a triple bond, or another suitable multiple bond, and a hydrogen bond, and the like.

Preferably, the chemical bond between the individual components is a covalent single bond. Preferably, the “link” or chemical bond between two components is a direct bond between the two components; but it may also comprise a suitable linker atom or molecule, if necessary. In some cases, one or both of the components to be linked will have to be activated in order to allow for the formation of a chemical bond or link. The procedures and means to be applied for such activation and formation of chemical bonds are standard knowledge of the field, and any skilled person will immediately know how to proceed.

Since the individual components are required to be linked (or chemically bonded) to each other as specified, the term “component” is used to indicate the respective parts of the probe compound which are characterised by their respective function as determined in the following. Herein, the term “component” is meant to refer to a specified part or moiety of a molecule, which is identified by its function within this molecule. For example, the term “indicator component” refers to a part or moiety of the molecule, which comprises a signal-generating function, e.g. a fluorescence dye or the like, that allows for indicating the location of the probe compound. Similarly, the term “test component” refers to a part or moiety of the molecule, which comprises a substrate or metabolite, thus being the site of enzymatic interaction with the probe compound. The respective components are linked by chemical bonds to form the molecule, i.e. the probe compound. It should be noted that, in the context of this invention, the term “component” is used with the meaning of “part” or “moiety” of one compound or molecule, and not to refer to individual members of a multi-molecule system. The terms “component”, “part” and “moiety” may be used alternatively with this meaning. However, for reasons of consistency, the term “component” will also be used to refer to the respective molecules before they are reacted to form the respective part (component) of the probe compound, or after they are released from the probe compound, respectively. A person skilled in the art will readily understand the exact nature of the respective molecules, as well as their contribution to the probe compound.

The term “linked” indicates the presence of at least one chemical bond between the corresponding components. A chemical bond can be a hydrogen bond, an ionic bond or a covalent bond, including a bond between a transition metal atom and its surrounding ligand atoms in a coordination compound (also referred to as “coordination bond” in the following). Preferably, the chemical bond(s) between the components of the probe molecule are covalent bonds, further preferred covalent single bonds, wherein the bond(s) between the reactive component and the transition metal complex preferably are coordination bonds. The probe compound of the invention is required to have bonds between the transition metal complex and the reactive component, as well as between the test component and the indicator component forming the reactive component. However, the respective components may also be linked by additional bonds, as long as such bonds do not hinder the function of the probe compound. For example, the reactive component may be linked to the transition metal complex by at least one coordination bond formed between the test component moiety of the reactive component and the central metal atom of the transition metal complex and by at least one coordination bond formed between the indicator component moiety of the reactive component and the central metal atom of the transition metal complex. In a preferred embodiment, the reactive component is linked to the transition metal complex by exactly one coordination bond formed between the test component moiety of the reactive component and the central metal atom of the transition metal complex and by exactly one coordination bond formed between the indicator component moiety of the reactive component and the central metal atom of the transition metal complex. The respective coordination bonds can be direct bonds between the metal atom and a suitable atom of the test or indicator components, or a bond formed via a suitable linker moiety, which is linked to the test and/or indicator component, respectively. A preferred linker moiety is a histidine residue. By forming links via a linker moiety, it is possible to obtain a reproducible binding property for all test and indicator components.

The term “test component” is used for the part or component of the probe compound which comprises a substrate or metabolite. Herein, the terms “substrate” and “metobolite” refer to any molecule capable of specific interaction with the active site of an enzyme. The substrate or metabolite is comprised in the test component in such a manner that its characteristic structure and/or functional groups necessary for interaction with the active site of an enzyme are maintained within the test component. Therefore, the substrate or metabolite is preferably linked to the other components of the probe compound at positions of the substrate or metabolite molecule, which are not involved in enzyme-substrate interaction. Optionally, a spacer or linker moiety can be used to provide a suitable binding position on the test component, which allows for an unimpeded enzyme interaction. In this case, the term “test component” refers to the component comprising both the substrate and the spacer moiety. The test component may be linked to the probe compound by chemical bonds involving positions, i.e. atoms or functional groups, of the substrate or metabolite and/or an optional spacer or linker moiety.

The term “test component” is preferably used to indicate a component comprising a so-called “small organic molecule” that can interact with an enzyme. The term “small organic molecule” refers to a molecule comprising of carbon and hydrogen atoms, optionally including nitrogen, oxygen, phosphorous, sulfur, and/or halogen (F, Cl, Br, I) atoms, and having a molecular weight of 5000 Da or less, preferably of 2000 Da or less. Preferably, the test component comprises a known substrate of at least one enzyme. Moreover, the test component can also comprise a pseudo-substrate or inhibitor of a known enzyme. However, the test component may also comprise a molecule suspected to interact with the active site of an enzyme. The test component may also comprise a small organic molecules for the search for pharmaceutical active ingredients. According to one embodiment, the “test component” does not comprise a polymeric compound based on nucleic acids, such as DNA, cDNA, RNA, or the like, or a polymeric compound based on amino acids, such as peptides, proteins, or the like. According to another embodiment, the test component may comprise at least one nucleic acid and/or amino acid, if necessary. Preferably, the test component comprises one or two nucleic acids, or one or two amino acids. According to another embodiment, the test component comprises a polymeric compound, such as a polymeric compound based on natural occurring sugar units or the like, e.g. cellulose or the like. Preferable, a polymeric compound has a molecular weight of 5000 Da or less, i.e. 5 kDa or less. According to another embodiment, the test component comprises a polymeric compound based on nucleic acids, such as DNA, cDNA, RNA, or the like, and/or a polymeric compound based on amino acids, such as peptides, proteins, or the like. Moreover, the test component can be preferably functionalised with a linker component or moiety suitable for binding to the transition metal complex. Such functionalisation is especially advantageous for test components comprising substrates, which do not readily form coordination bonds. In a preferred embodiment, the test component is functionalised with a histidine molecule or residue (sometimes referred to as a “His-tag” in the following). The amino acid histidine was found to be especially versatile, because it may be linked to a test component or indicator component by either its amine function or its carboxylic acid function, while the imidazole ring provides for a good coordination bond to the transition metal atom. A histidine residue may be linked to the substrate or metabolite comprised in the test component or to another part of the test component, either directly or using a suitable linker moiety. Similarly, a histidine residue may be linked to the dye comprised in the indicator component or to another part of the indicator component, either directly or using a suitable linker moiety. A skilled person will know how to link a histidine residue to the desired site or position of a test or indicator component, whether a special activation and/or linker moiety will be required, as well as the starting materials and conditions necessary therefor, etc. A His-tag has the additional advantage to ensure an identical binding property of all possible substrates to the transition metal complex. Moreover, it was found that a link including a His-tag may be advantageously broken upon enzymatic reaction of a test component comprising a His-tag. Moreover, it was found that by selecting the site of histidine binding to the test component, it is possible to prepare probe compounds which allow for the identification of different metabolic pathways and the enzymes involved therein.

The term “indicator component” is used to indicate a molecule which can generate a signal, thus indicating the location of the probe molecule, e.g. on an array. That signal can either be produced directly, for example by absorbance or fluorescence, or can be generated in further treatments with detection reagents, for example in the form of an optical or radioactive signal. Preferably, the indicator component comprises a dye, further preferred a fluorescence dye. The term “fluorescence dye” indicates a molecule showing fluorescence upon irradiation with a suitable light source. Preferably, an indicator component comprises a fluorescent azo compound or a cyanine compound, or the like. Especially preferred is a cyanine compound, which is known in the art under the name of “Cy3”. If necessary, the moiety having the fluorescence property is further modified, e.g. by addition of a suitable linker moiety, in order to allow the binding to the test component, and/or to the transition metal complex. For example, a Cy3 dye available as its Cy3-NHS-ester may be reacted with both histidine and a 4-amino-3-butyric acid linker to allow for linking with both the transition metal complex and the test component, respectively. An additional linker moiety has the advantage to ensure an identical and reproducible binding to the indicator component and/or the transition metal complex, which allows for the ready use in parallel synthesis or the like.

The test component is linked to the indicator component to form the reactive component. Preferably, the test component is linked to the indicator component by at least one covalent chemical bond, further preferred by one, two, or three covalent bonds, and still further preferred by exactly one covalent bond. A preferred example of such a covalent bond is a carbon-oxygen single bond, which can be formed between the test component and the indicator component by various chemical reactions, such as, for example, a condensation reaction, an addition reaction, an oxidation reaction, and the like. A preferred example is a condensation reaction between a carboxylic acid and an alcohol, or between two alcohols, or an addition reaction wherein the oxygen atom of an alcohol function is added to an aliphatic or aromatic carbon atom, or the like. It should be noted that such bond forming reaction is not restricted to the examples given above, and that there is no prejudice regarding which individual function should be present in the respective molecules to become the respective components, etc. A person skilled in the art will immediately know which combinations of functional groups will be required to form a corresponding chemical bond between the individual molecules to become the respective components of the probe compound, and also which starting materials and conditions etc. are required to form the desired chemical bond between the components. Another preferred covalent bond is a carbon-nitrogen single bond. Preferably, the carbon-nitrogen single bond is part of a quaternary amine function (quaternary ammonium function) comprised in the link between test component and indicator component. The link or bond between the test component and the indicator component may also be formed between the test component and a linker moiety previously attached to the indicator component, or vice versa. An additional linker moiety has the advantage to ensure an identical and reproducible binding to the test component, which allows for the ready use in parallel synthesis or the like. In a preferred embodiment, the indicator component comprises an amino butyric acid linker moiety, and a bond or link to the test component is formed using the carboxylic acid function of this linker moiety. Preferably, the link between indicator component and test component comprises a linker moiety comprising an amino butyric acid linker residue attached to the indicator component, the carboxylic acid function of which is linked to another linker moiety comprising a quaternary amine function, which in turn is linked to the test component. Such linker moiety can be formed, for example, by reacting an indicator component, e.g. a fluorescence dye, first with 4-amino-3-butanoate and then with N,N-dimethylethanolamine. Linking with the test component under formation of a quaternary amine can then be obtained by reacting the so-prepared indicator component having a linker moiety comprising an amino butyric acid residue, the carboxylic acid function of which is esterised by N,N-dimethylethanolamine, with a iodine-containing test component in the presence of 1,8-bis-(dimethylamino)-naphthalene, or a similar method.

The reactive component comprising the test component and the indicator component, which are linked to each other, optionally by a linker moiety or molecule, is in turn linked to the transition metal complex. Preferably, the reactive component is linked to the transition metal complex by two, three or four coordination bonds, and further preferred by exactly two coordination bonds. That means that at least one atom of the reactive component is a direct ligand atom of the transition metal atom of the transition metal complex, thus being part of the immediate coordination sphere of the central transition metal atom of the transition metal complex. The term “at least one atom of the reactive component” also includes an atom of a linker moiety, which can optionally be attached to either the test component or the indicator component. For example, in a preferred embodiment, a histidine molecule is attached as a linker moiety (His-tag) to the reactive component. In this case, the “at least one atom of the reactive component” can also indicate an atom of the His-tag, preferably a nitrogen atom of the imidazole ring system. Preferably, the reactive component is linked to the transition metal complex by two coordination bonds, wherein one ligand atom is an atom of the test component and the other ligand atom is an atom of the indicator component. In a preferred embodiment, the reactive component comprises a His-tag linked to the test component and a His-tag linked to the indicator component. In this case, the reactive component is linked to the transition metal complex by two coordination bonds, wherein each bond includes one atom of one of the respective His-tags.

A preferred coordination bond between the reactive component and the transition metal complex is a M-O—R-bond, wherein M indicates the transition metal atom and O—R indicates an oxygen atom or function of a molecule R constituting the reactive component. Preferred examples of suitable oxygen functions are an alcoholate function (R—O⁻), a carboxylate function (RCOO⁻), a peroxide function (R—O—O⁻), or the like. Another preferred coordination bond between the reactive component and the transition metal complex is a M-N—R-bond, wherein M indicates the transition metal atom and N—R indicates a nitrogen atom or function of a molecule R constituting the reactive component. An example for such nitrogen function is an aliphaptic amine function, including a primary, secondary and tertiary amine function. The nitrogen atom can also be part of an aromatic or (partially) saturated ring system, such as, for example, a pyrrol, imidazole, diazole or triazole ring, or the like. A specially preferred coordination bond is a coordination bond comprising a nitrogen atom of an imidazole ring, which may be part of a histidine moiety or His-tag. Another preferred coordination bond is a M-S—R or a M-C—R single bond, wherein M indicates the transition metal atom and S and C, respectively, indicate a sulphur or carbon function of a molecule R constituting the reactive component. It should be noted that the coordination bond between reactive component and transition metal complex is not restricted to the examples given above, and that there is no prejudice regarding which individual function should be present in the respective molecules to form the desired coordination bond, etc. A person skilled in the art will immediately know which functional groups will be required to form a corresponding coordination bond, and also which starting materials and conditions etc. are required.

The transition metal complex preferably comprises at least one transition metal atom and at least one ligand molecule, wherein at least one coordination bond is formed between the ligand molecule and the transition metal atom. The at least one ligand molecule preferably comprises one or more atoms or functions which can form a coordination bond with a transition metal atom. Preferred examples for atoms or function are an oxygen atom, a nitrogen atom, a phosphorous atom, a sulphur atom, or the like. These atom or functions all can form a coordination bond with a transition metal atom, and are the same as exemplified above. Preferably, a ligand molecule comprises more than one atom or function that can form a coordination bond with a transition metal atom, further preferred two to eight of such atoms or functions, still further preferred three to five of such atoms or functions, and most preferred four or five of such atoms of functions. In a preferred embodiment of the present invention, the transition metal complex comprises exactly one transition metal atom and exactly one ligand molecule comprising more than one atom or function that can form a coordination bond with a transition metal atom. Herein, the term “transition metal atom” is used to indicate the central transition metal atom of the transition metal complex, which is linked to both the ligand molecule(s) and the reactive component by coordination bonds as defined above. Examples of suitable transition metal atoms are Ti, V, Mn, Fe, Co, Ni, Cu, Zn, Mo, W, Pt, Au, or the like. Preferred examples are Co, Ni, and Cu. The transition metal atom means any transition metal atom irrespective of its oxidation state. The term transition metal ion is also used for a transition metal atom that carries a net charge, which is sometimes referred to as a “transition metal ion” in the art, wherein a formal charge or oxidation state is assigned to the transition metal atom or ion. Preferred oxidation states of transition metal atoms of the present invention are from 0 to +4, preferably +2 to +3, and especially preferred +2. Preferred examples of transition metal atoms are Co(II), Ni(II), and Cu(II). Accordingly, the coordinating atoms or functions of the ligand molecule may be assigned with a formal charge, wherein preferred charges range from 0 to −2, wherein a charge of 0 or −1 is especially preferred. As used herein, the term “ligand molecule” refers to a molecule that comprises at least one atom or function which forms a direct coordination bond to the central atom of a transition metal complex, preferably one, two, three, four, five, six, seven, or eight such atoms or functions. It should be noted that the coordination bond between ligand molecule and transition metal atom is not restricted to the examples given above, and that there is no prejudice regarding which individual function should be present in the ligand molecule to form the desired coordination bond, etc. A person skilled in the art will immediately know which functional groups will be required to form a corresponding coordination bond, and also which starting materials and conditions etc. are required.

The term “probe compound” indicates a compound or molecule that can interact with an enzyme (or analyte molecule) in a reactive manner, as outlined in the following. The term “interaction with an enzyme in a reactive manner” indicates an interaction wherein the reactive component is not only bound to the enzyme, but also transformed in a reaction catalysed by the enzyme (metabolised). The reaction catalysed by the enzyme can be any reaction catalysed by an enzyme, such as, for example, an oxidation or reduction reaction, an addition reaction, a hydrolytic bond cleaving reaction, an elimination reaction, an isomerisation reaction, or a condensation reaction.

Preferably, the reaction catalysed by the enzyme is a hydrolytic cleaving reaction or an elimination reaction, wherein the enzyme cleaves the link between the test component, or its reaction product, respectively, and the indicator component and/or the transition metal complex.

Preferably, the interaction with the enzyme results in a cleavage of the link between the test component and the transition metal complex by the enzyme, whereupon a reaction product comprising the metabolised test component and the indicator component together is formed. Further, the interaction with the enzyme may also result in a cleavage of both the link between the test component and the transition metal complex and the link between the test component and the indicator component by the enzyme, whereupon more than one reaction products comprising the metabolised test component and/or the indicator component in separate molecules, may be released from the probe compound. For example, in a preferred embodiment wherein the test component is linked to the transition metal complex by a histidine moiety (His-tag), the link between the histidine is cleaved by the enzymatic reaction. As a result, the reaction product comprising both the test component and the indicator component remains linked to the transition metal complex, and thus to the remainder of the probe compound. The test component or the reaction product thereof may remain bound to the active site of the enzyme, thus immobilising the enzyme to the probe compound, or its reaction product, respectively. Moreover, the signal characteristic of the indicator component, which is triggered by the enzymatic reaction, also remains at the probe compound.

Alternatively, the interaction with the enzyme results in a cleavage of the link between the indicator component and the test component by the enzyme, resulting in a reaction product comprising the indicator component alone, or a reaction product comprising the metabolised test component and the indicator component together, or more than one reaction products comprising the metabolised test component and/or the indicator component in separate molecules. Here, the term “reaction product comprising the indicator component (or test component)” indicates a compound or molecule based on the indicator component or test component, respectively, which is metabolised and/or released by the corresponding reaction catalysed by the enzyme.

Alternatively, the interaction with the enzyme results in a cleavage of the both the link between the test component and the transition metal complex and the link between the test component and the indicator component by the enzyme, whereupon a reaction product comprising the metabolised test component, or more than one reaction products comprising the metabolised test component and/or the indicator component in separate molecules, may be released from the probe compound. For example, in a preferred embodiment wherein the test component is linked to the transition metal complex by a histidine moiety (His-tag), the link between the histidine is cleaved by the enzymatic reaction, and the link to the indicator component is cleaved as well.

Preferably, the reaction product comprising the indicator component is not released from the probe compound, i.e. the reaction product comprising the indicator component remains part of the probe compound, or its reaction product, respectively. For example, in a preferred embodiment wherein the indicator component is linked to the transition metal complex by a histidine moiety (His-tag), the histidine remains linked to both the transition metal complex and the indicator component upon enzymatic cleavage of the link between the test component and the indicator component. As a result, the reaction product comprising the indicator component remains linked to the transition metal complex, and thus to the remainder of the probe compound. Thereby, the signal characteristic of the indicator component, which is triggered by the enzymatic reaction, also remains at the probe compound.

Preferably, the reaction product comprising the test component is not released from the probe compound. For example, in a preferred embodiment wherein the test component is linked to the transition metal complex by a histidine moiety (His-tag), only the link between the histidine and the test component is cleaved by the enzyme. As a result, the reaction product comprising both the test component and the indicator component remains linked to the transition metal complex, and thus to the remainder of the probe compound. Thereby, the test component or the reaction product thereof may remain bound to the active site of the enzyme, thus immobilising the enzyme to the probe compound, or its reaction product, respectively. Moreover, the signal characteristic of the indicator component, which is triggered by the enzymatic reaction, also remains at the probe compound.

Preferably, the interaction of the probe compound with an enzyme results in the cleavage of both the links between transition metal complex and the reactive component as well as the link between the test component and the indicator component. This in turn exposes the transition metal atom which then ligates the enzyme, which can be used to immobilize the enzyme on an array spot or the like. The released indicator component has a changed binding situation which may preferably result in the generation of a signal.

The exact nature of all reaction products has not been revealed yet, but it is observed that an enzyme-specific reaction of the probe compound of the invention results in binding of the enzyme to the transition metal complex and the generation of a signal by the indicator component, e.g. the generation of a fluorescence signal.

It was found that the probe compound of the present invention allows for the detection of enzyme concentrations which are as low as 1.5 ng/ml protein or 2.5 pmol/ml substrate, respectively.

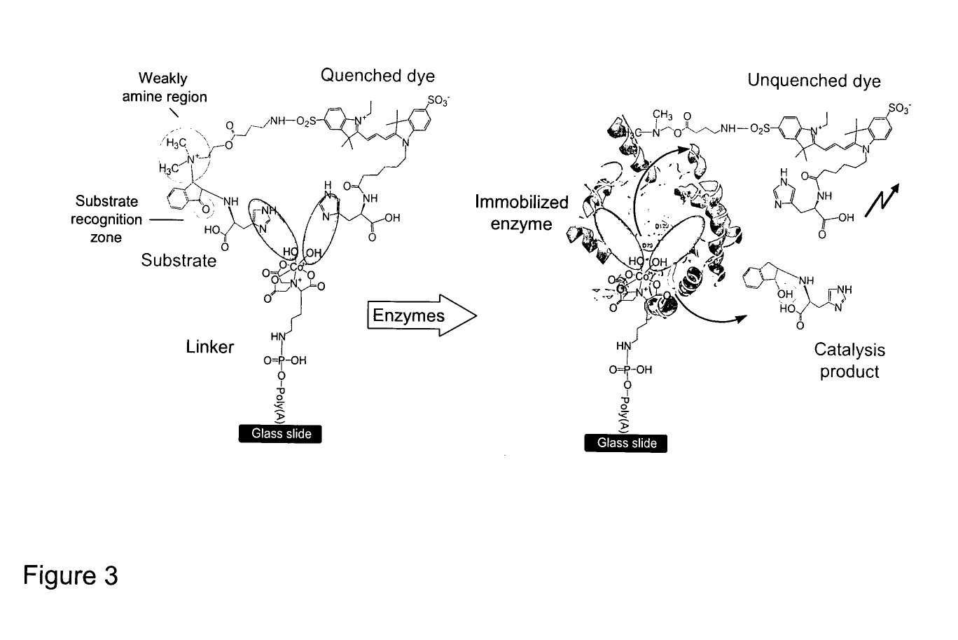

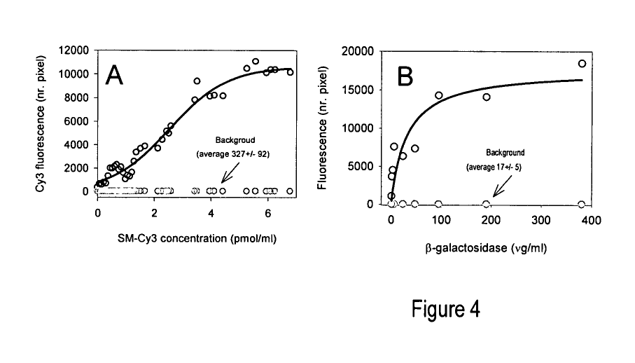

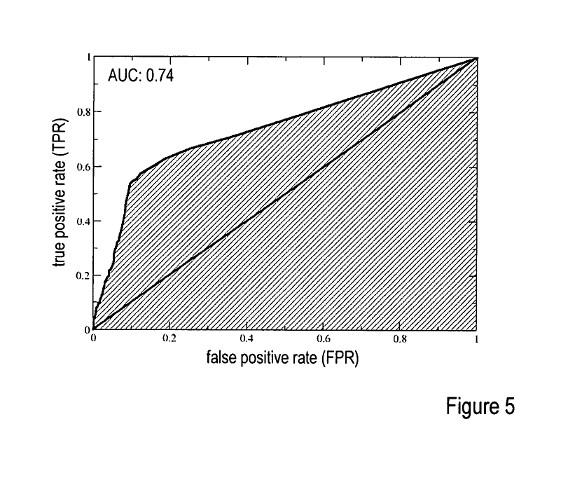

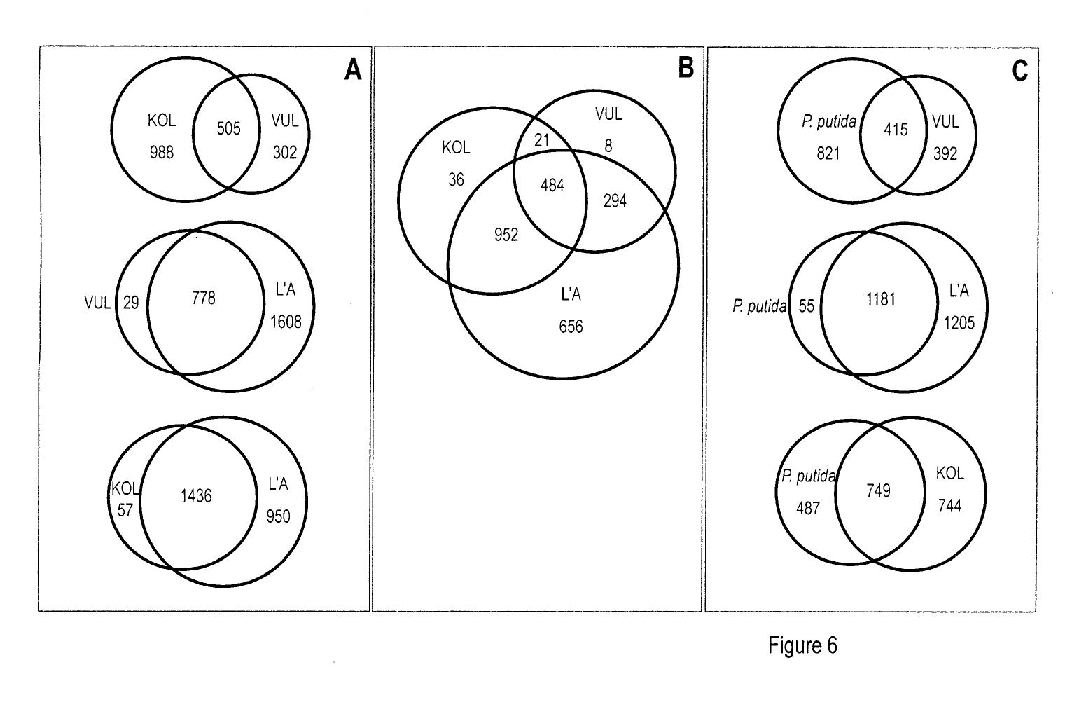

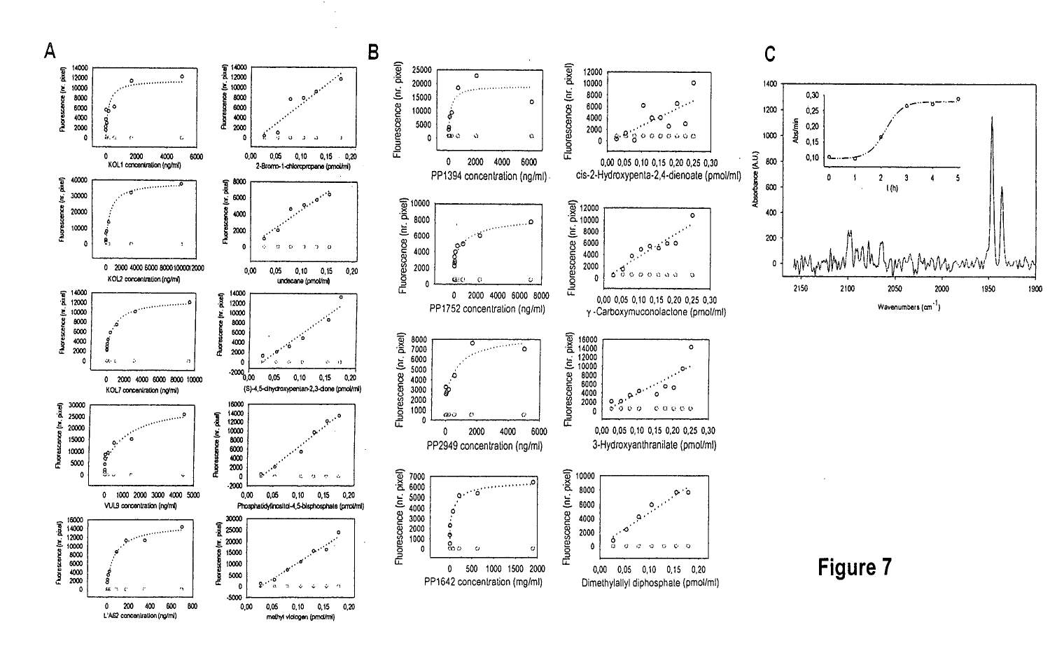

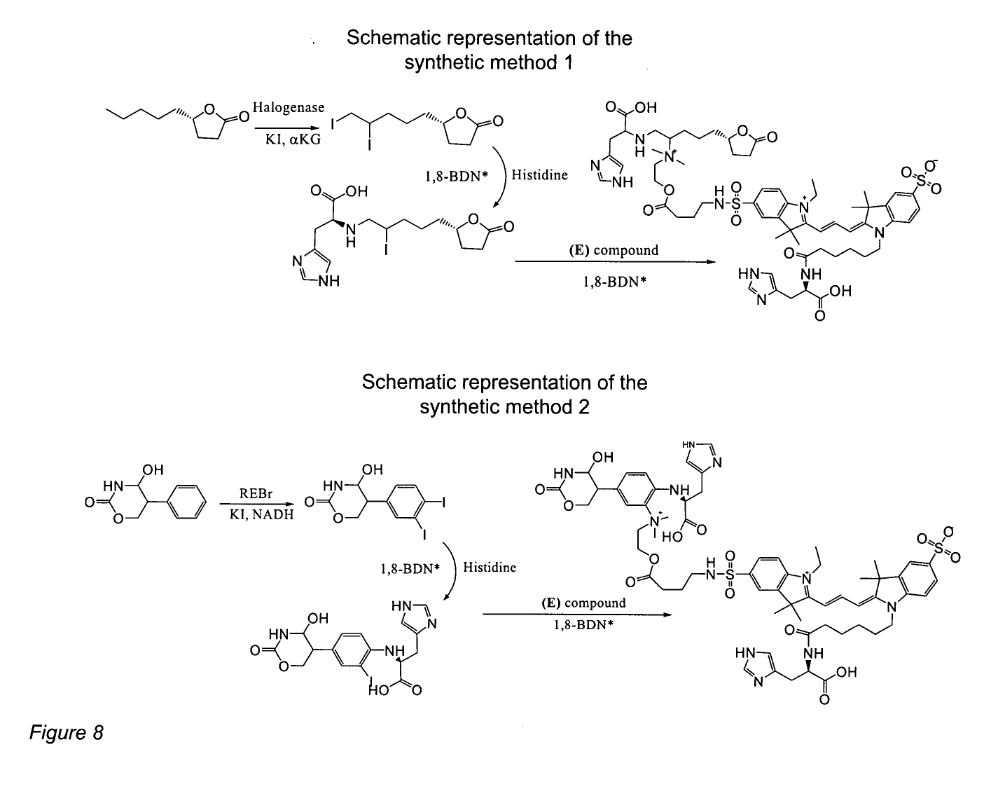

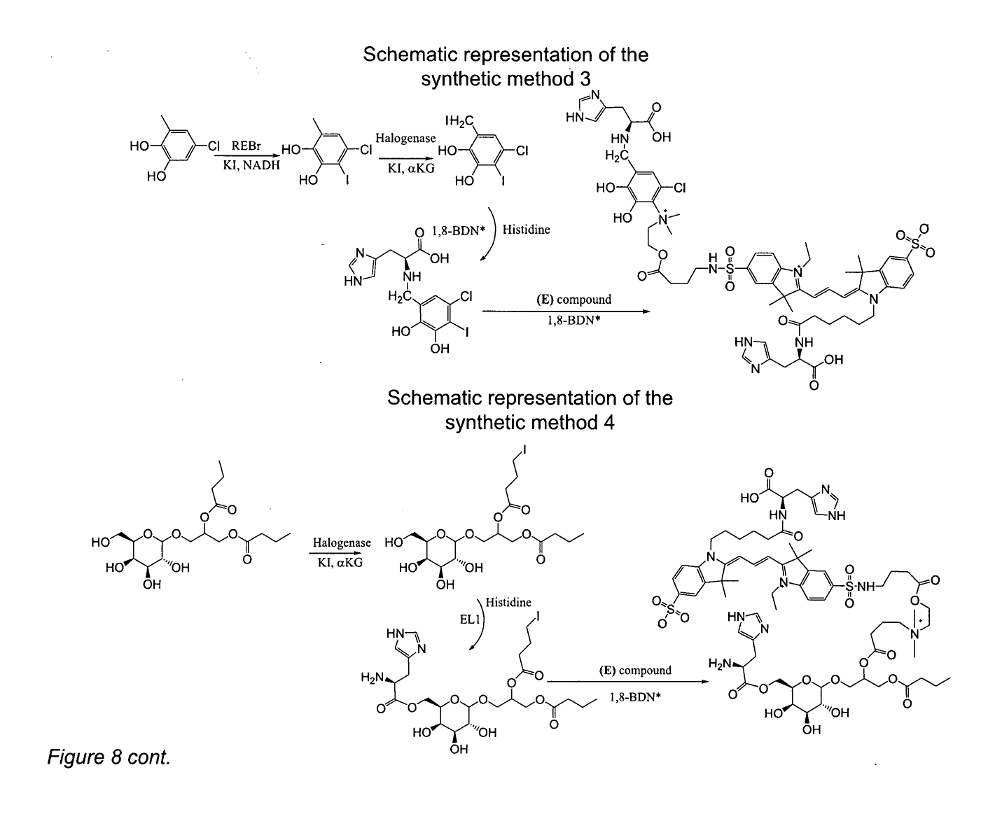

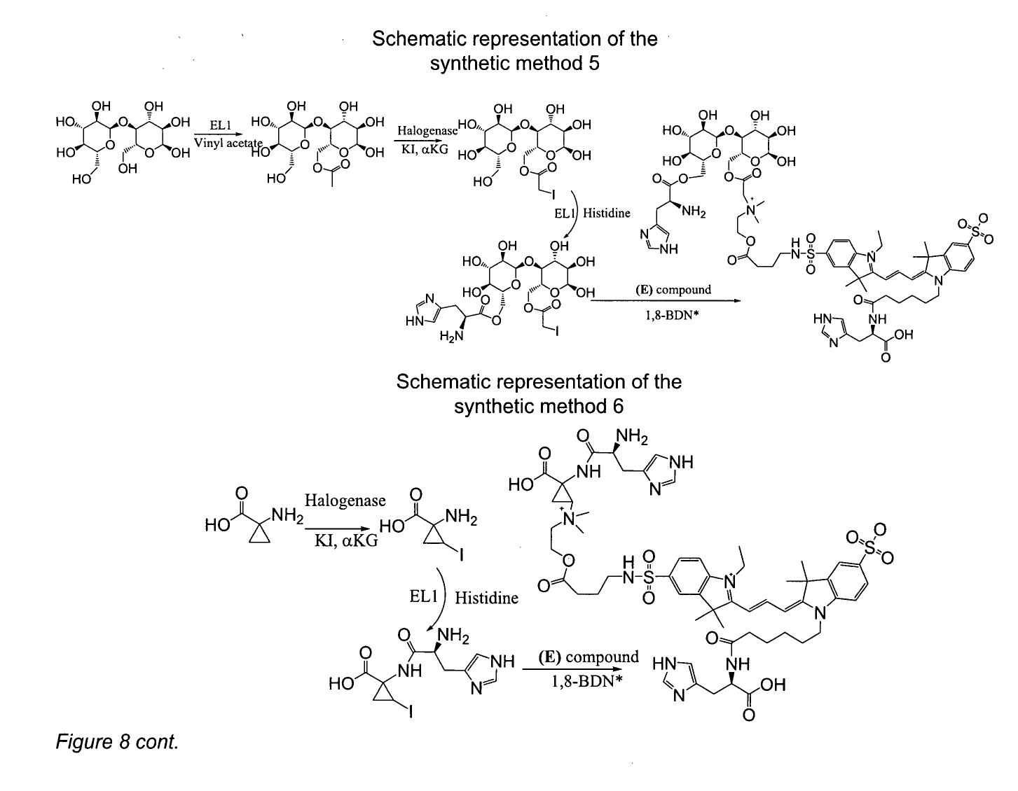

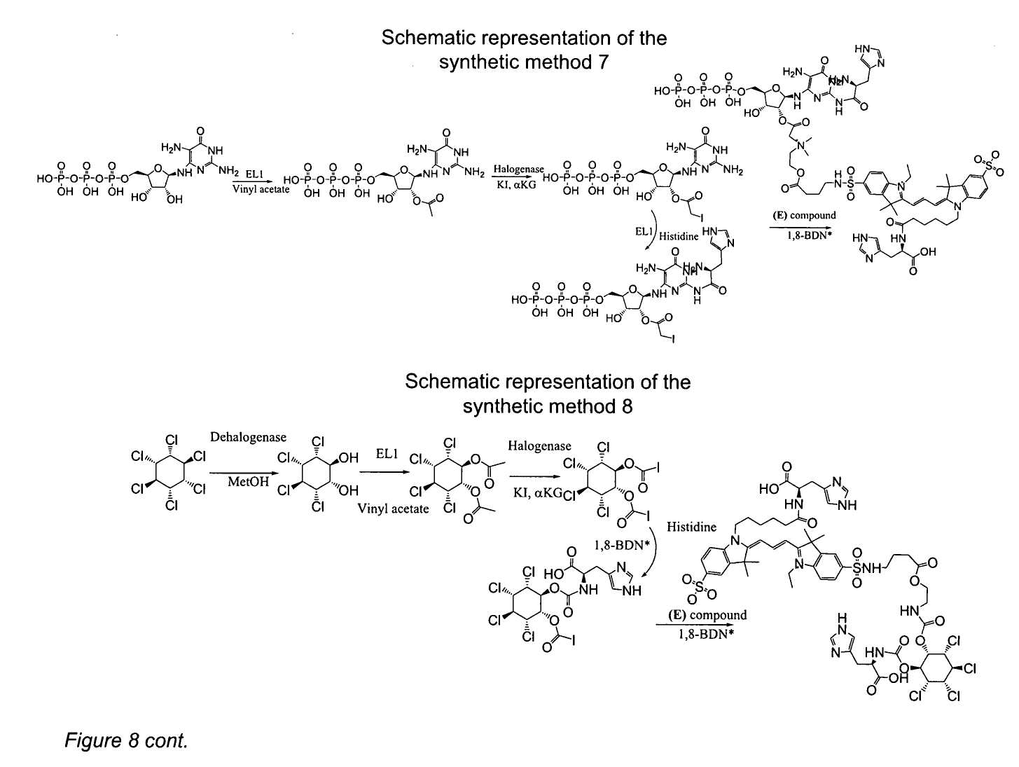

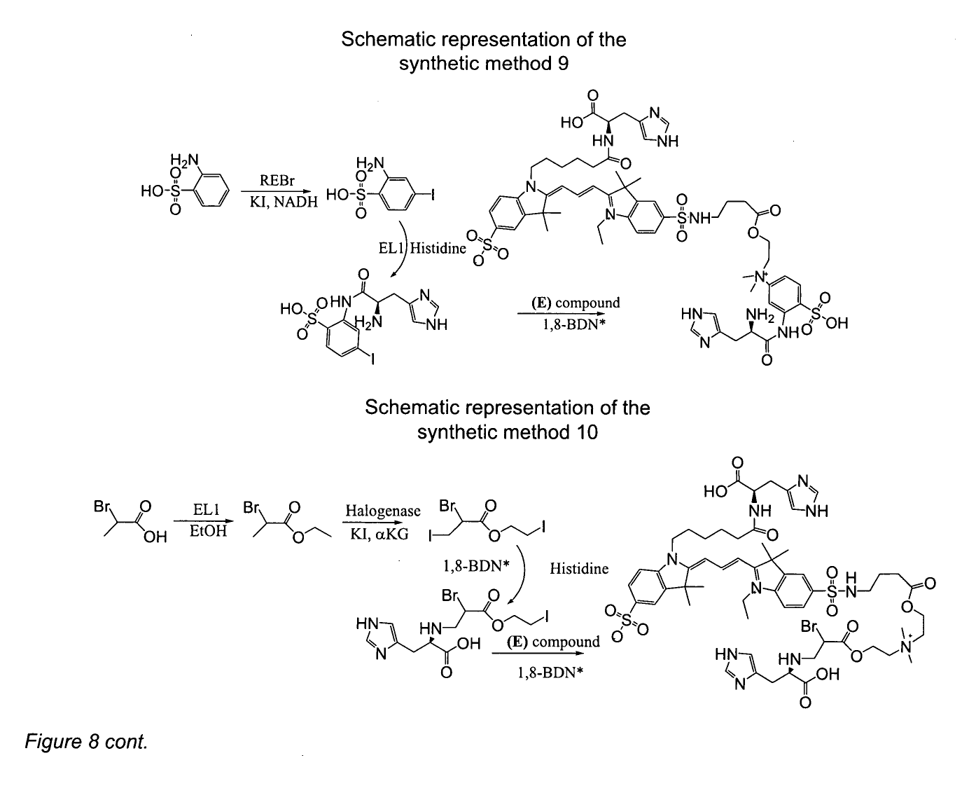

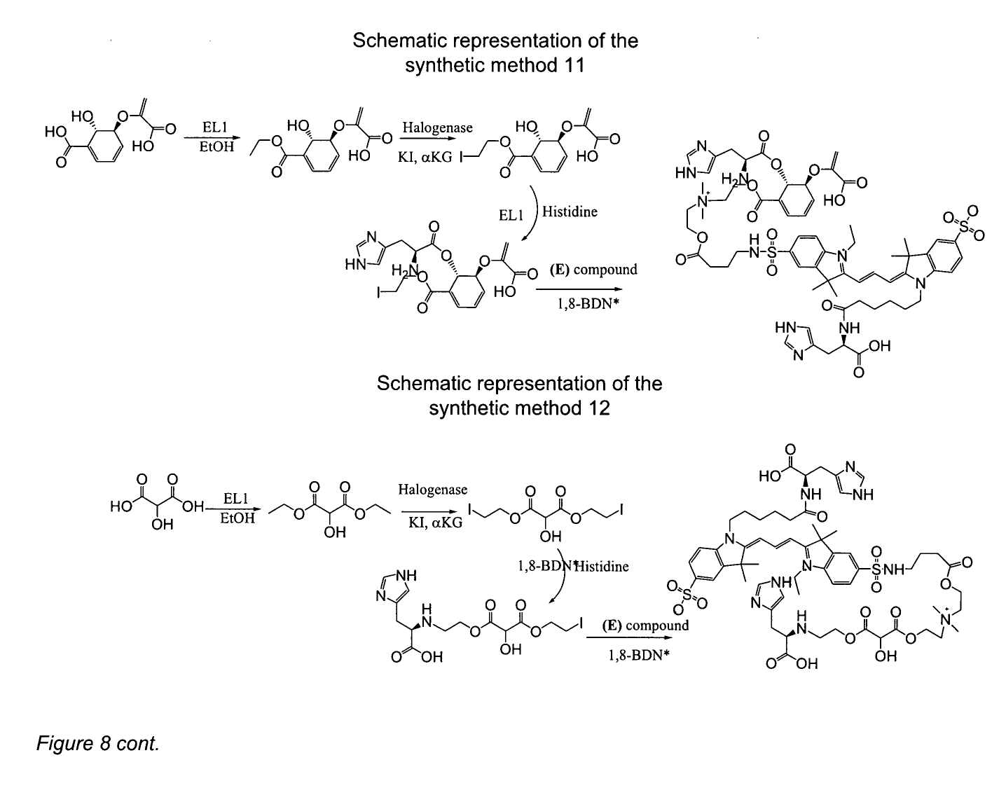

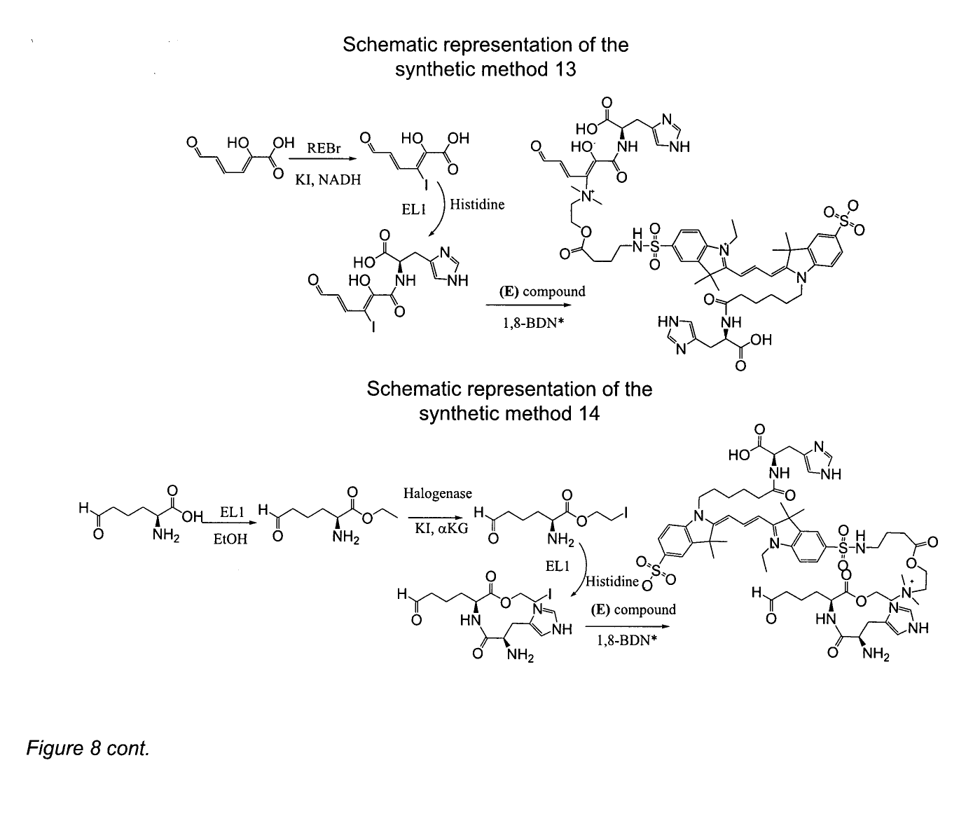

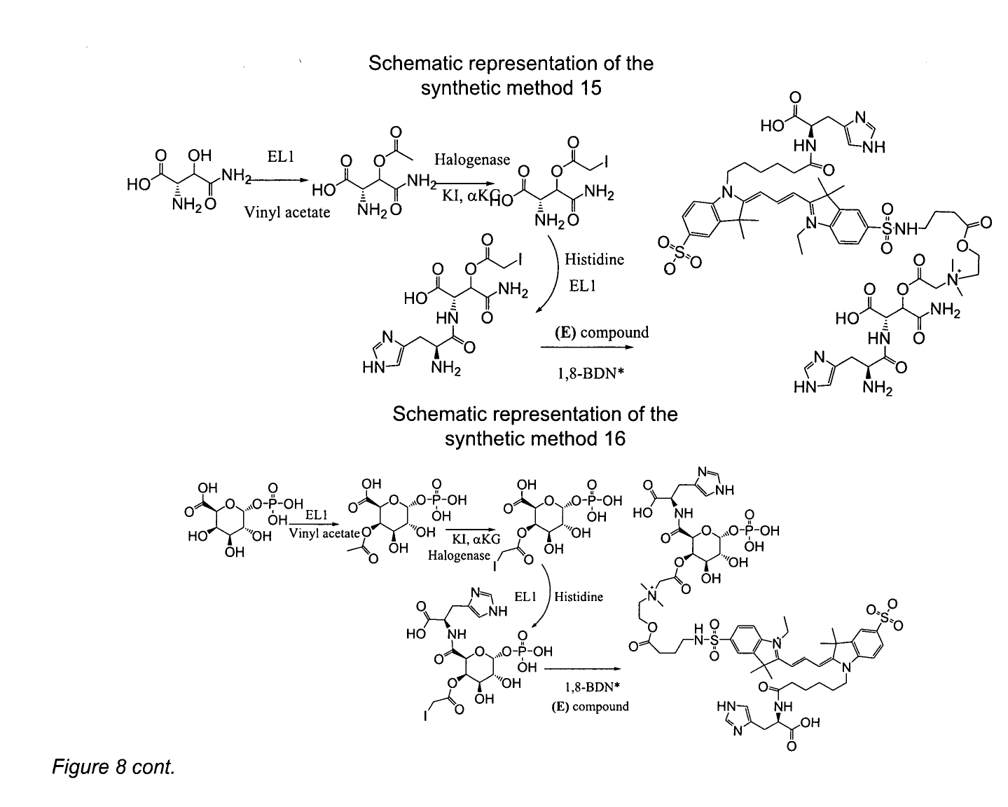

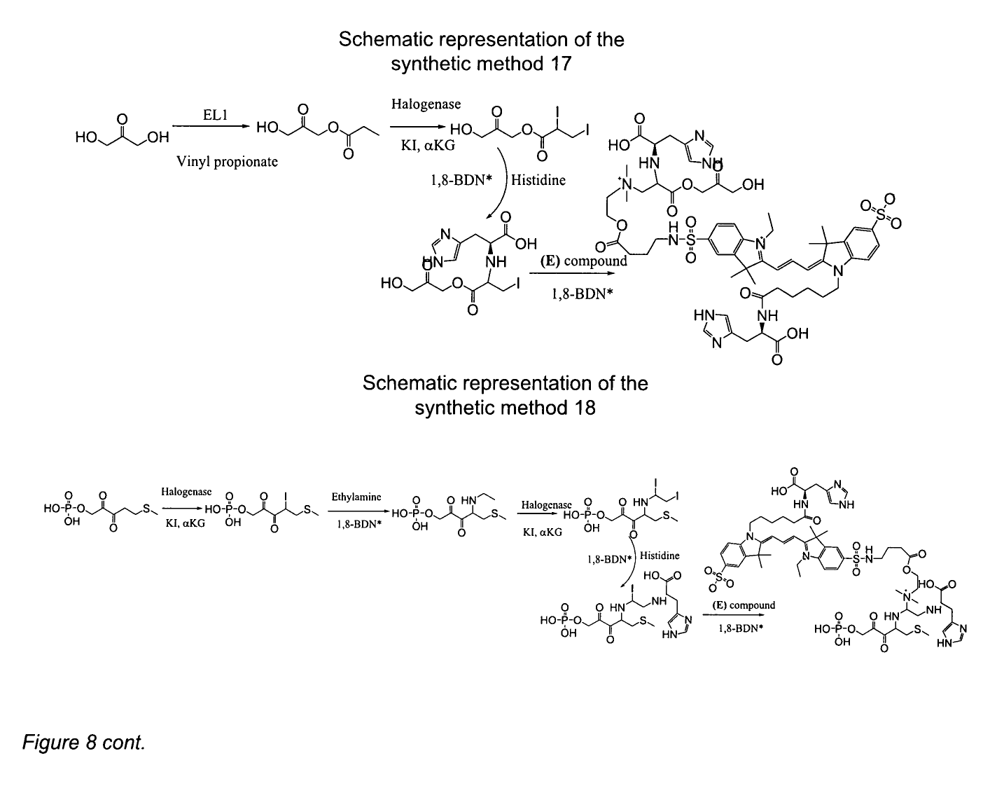

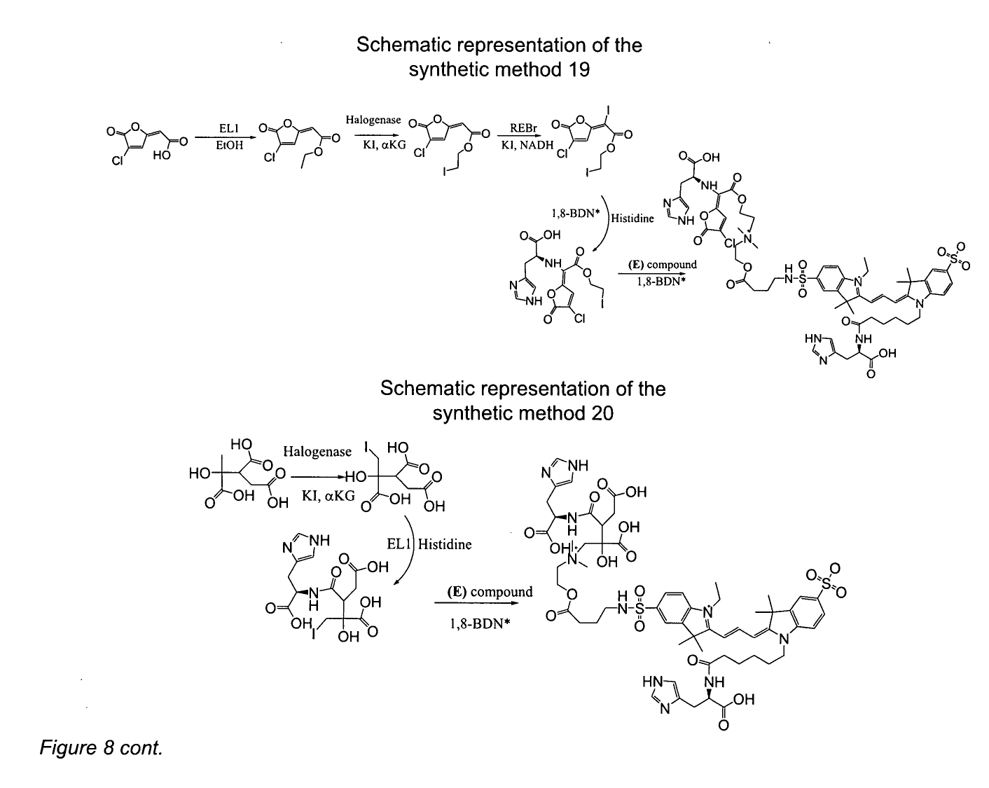

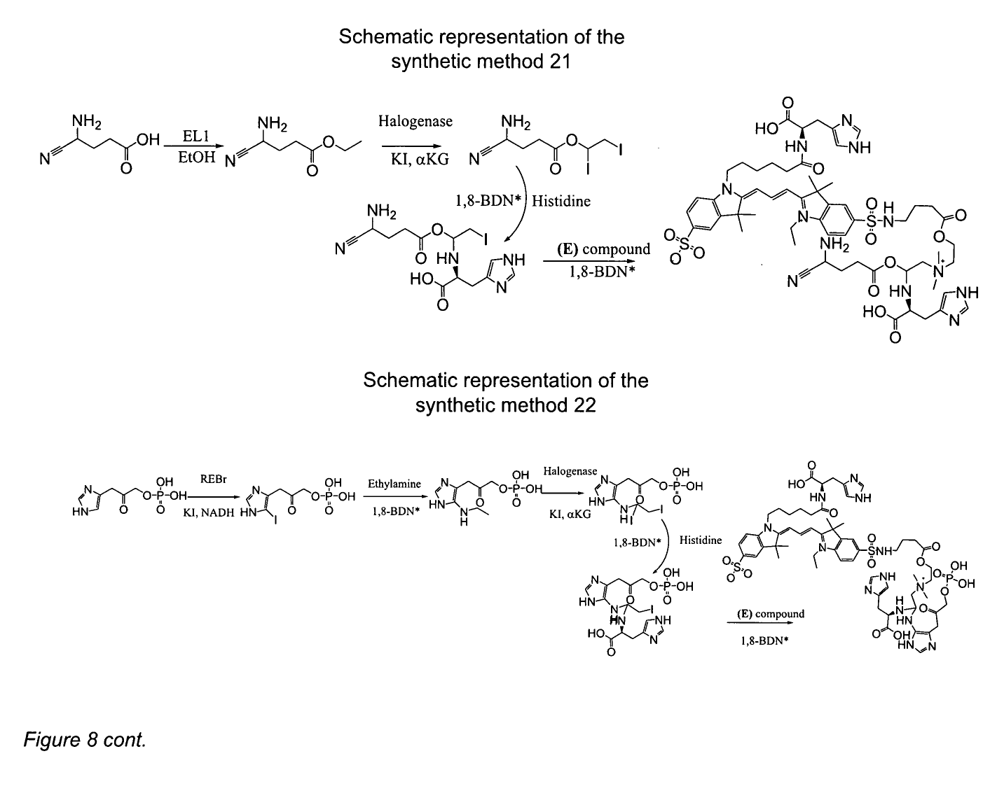

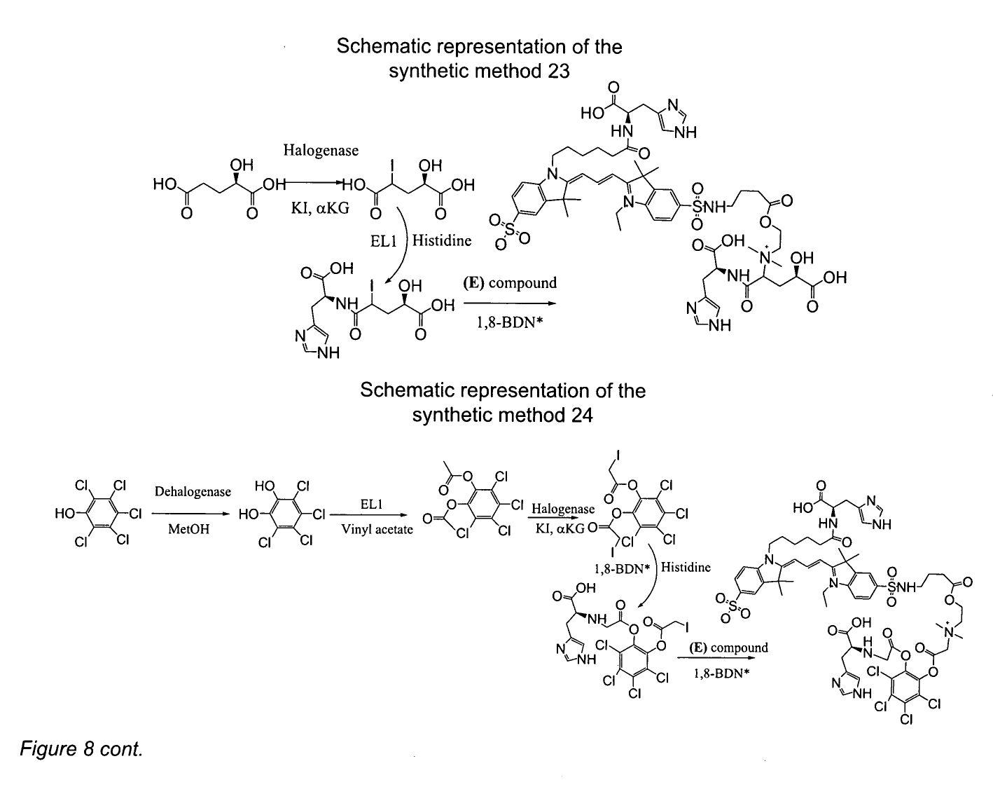

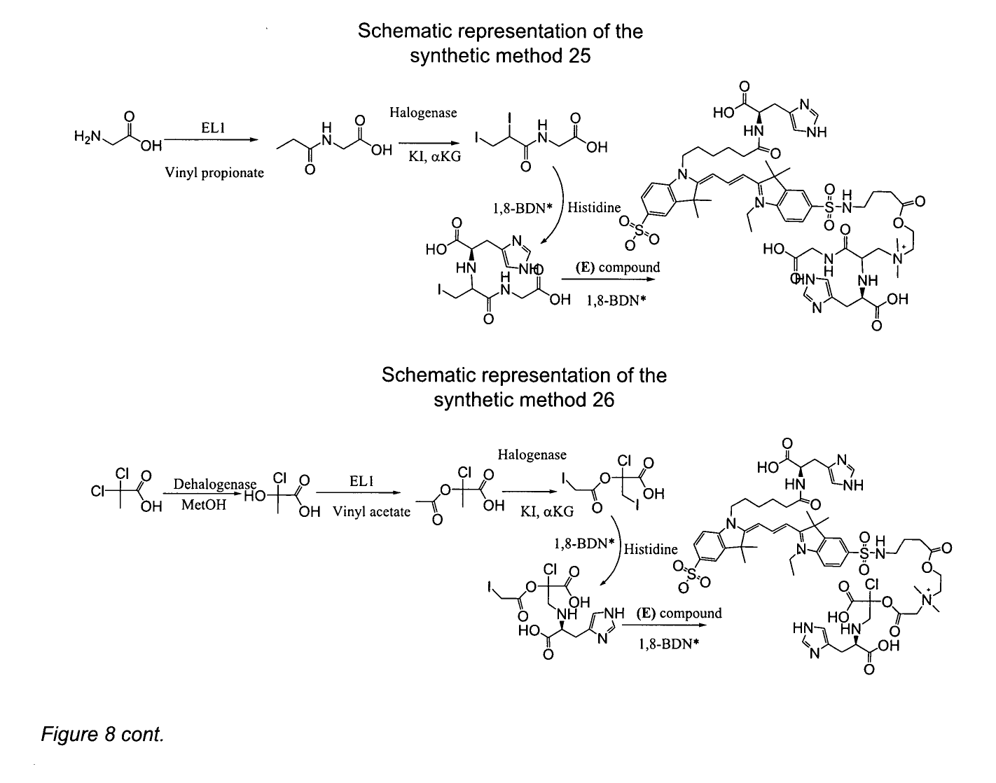

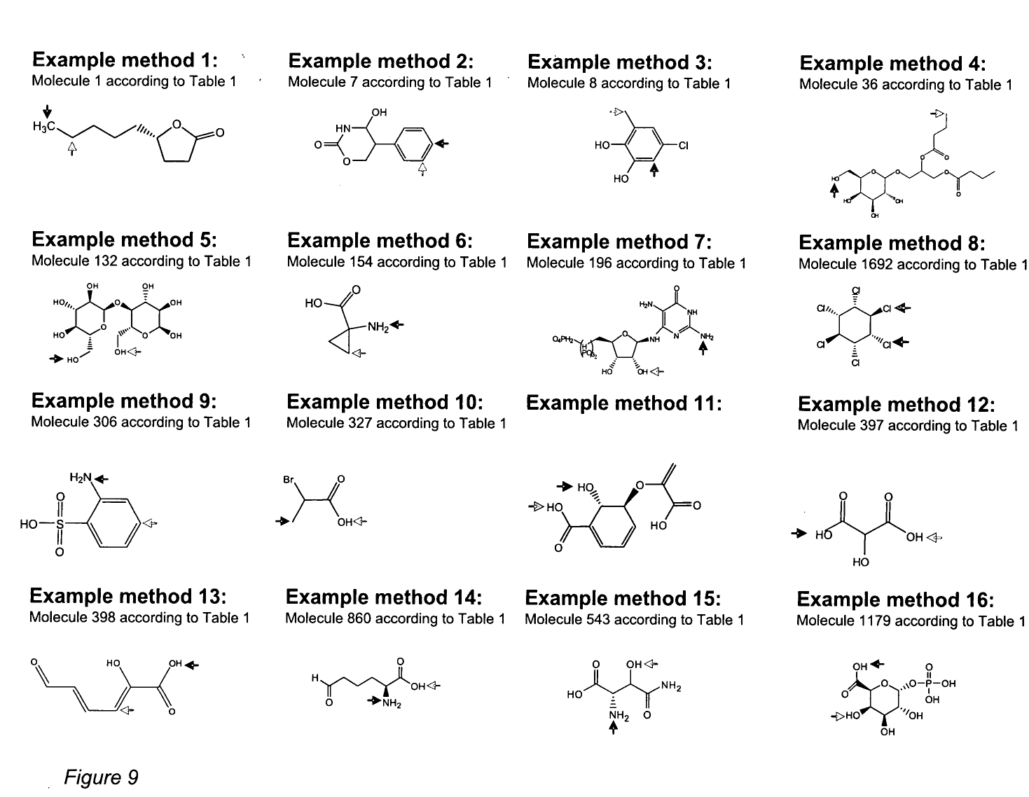

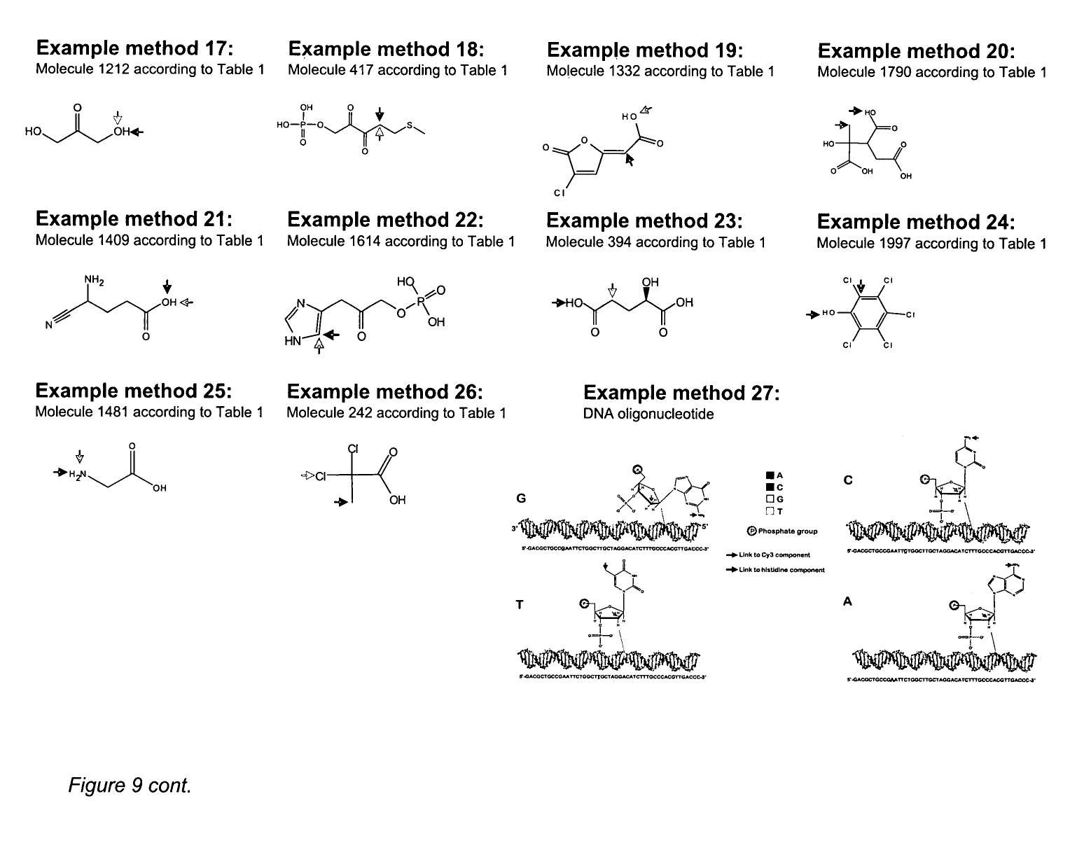

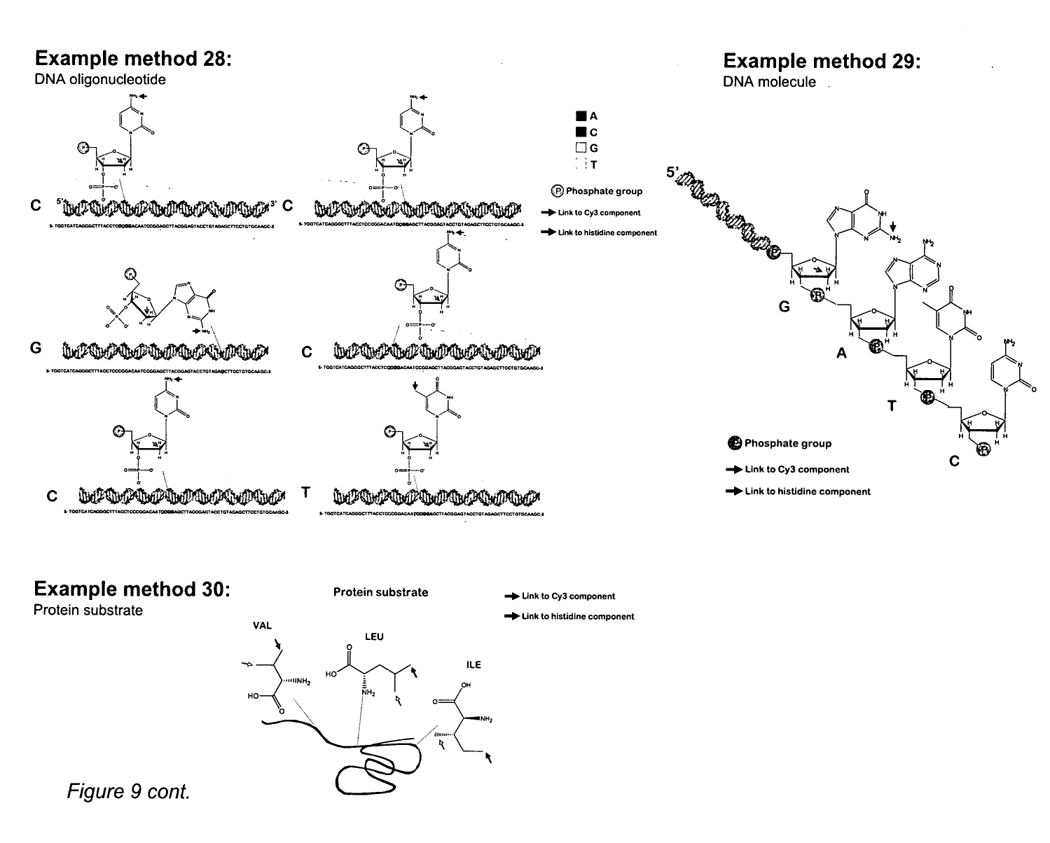

It was found that the probe compound of the invention advantageously allows for the testing of a reactive interaction of an enzyme with a small molecule or enzymatic substrate. The presence of the central transition metal complex further allows to provide a probe compound wherein all small molecules or substrates necessary for the life functions of an organism or communities living in a habitat can be readily included. Information about substrates that are involved in one or more metabolic reactions and about the enzymes involved in the corresponding metabolic reactions may be found, for example, in the Kyoto Encyclopedia of Genes and Genomes (KEGG Database), the University of Minnesota Biocatalysis and Biodegration Database (UM-BBD), PubMed, or the like. The probe compound of the invention was shown to be highly versatile for the identification of the reactive interaction with any small molecule or substrate tested so far. The key characteristic of the probe compound is that a productive reaction with a cognate enzyme releases the indicator component, producing a detectable signal, e.g. a fluorescent signal, and simultaneously results in the capture of the reacting enzyme through coordination with the transition metal complex. Non-productive interactions of proteins with the probe compound, such as, for example, binding without chemical reaction, do not lead to the release of the indicator component and the associated production of a detectable signal. An example of this rectional behaviour is shown in FIG. 3. The probe compound provides a highly sensitive, accurate, reproducible, and robust high-throughput tool for a genome-wide analysis of the metabolic status of an organism or community. Advantageously, the complete reactome (i.e. the complement of metabolic reactions of an organism) can be provided without prior knowledge of its sequence in as little time as 30 minutes or less.

Preferably, the transition metal complex comprises a cobalt or copper atom, and most preferred a cobalt atom. It was found that a cobalt or copper complex shows an advantageous binding property to all reactive components of interest, thus allowing for a most versatile use of the probe component of the invention. Especially preferred is a cobalt complex wherein the central cobalt atom is assigned a formal oxidation state of +2.

Preferably, the transition metal complex comprises a multidentate ligand molecule, i.e. a ligand molecule comprising two or more ligand atoms bound to the central transition metal atom. Further preferred, the transition metal complex comprises a multidentate ligand molecule comprising two, three, four, five, or six ligand atoms bound to the central transition metal atom. Especially preferred, the transition metal complex comprises a multidentate ligand molecule whose coordinating ligand atoms do not occupy all possible coordination positions of the central transition metal atom. For example, in the case of a central cobalt atom (Co²⁺), which usually exhibits coordination numbers of five or six, the multidentate ligand may occupy five, four or three of the potential coordination (binding) positions. Thus, a preferred ligand molecule for a central cobalt atom should provide three, four, or five coordinating ligand atoms, especially preferred four coordinating ligand atoms. A preferred example for a ligand molecule is a molecule comprising at least one coordinating nitrogen atom, such as, for example, a nitrogen atom of an aliphatic amine function, and at least one coordinating oxygen atom, such as, for example, an oxygen of a carboxilic acid function. A preferred example for a ligand molecule comprises nitrotriacetic acid. For example, in the case of a central cobalt atom, a ligand molecule based on nitrotriacetic acid will occupy four of the five or six potential coordination or binding positions, thus leaving one or two free binding positions for binding of the reactive component. However, a person skilled in the art will readily know which other ligand molecules exhibit similar properties and thus can also be used in the present invention.

Preferably, the transition metal complex comprises an anchoring component. The term “anchoring component” indicates a molecule or function that can be used to attach the probe compound to a solid surface, or to another component or molecule, or the like. Thus, the transition metal complex, and thereby the whole probe compound, can be attached to any suitable solid surface, or component or molecule, known in the art. Suitable solid surfaces may be porous surfaces, such as, for example, paper or cellulose substrates or the like, or non-porous surfaces, such as, for example, glass surfaces, or surfaces of polymeric materials, such as a surface of polycarbonate, or the like. For example, the anchoring component can be used to attach the probe compound to the surface of a glass slide in order to form an array thereon. Alternatively, the anchoring component can be used to attach the reactive compound to another component, such as, for example, a nanoparticle. The anchoring component may be any molecule or function known in the art which has the desired function to allow for the attachment to a solid surface, another component or molecule, or the like. A person skilled in the art will readily know which molecule or function should be used for a desired attachment. Preferably, the anchoring component comprises a polymeric compound, further preferred a polymeric compound of a biomolecule. A specially preferred anchoring component comprises a poly A chain, i.e. a polymer of adenosin. Any poly A chain known in the art may be used. Preferably, the poly A chain has a molecular weight of from 10 kDa to 150 kDa, further preferred of from 50 kDa to 120 kDa, and most preferred of about 100 kDa. A poly A chain is known in the art for binding to glass surfaces, activated silica, or the like. A poly A chain can be easily attached to a glass surface or the like, which might be optionally activated, and polymerised thereon using UV radiation according to standard protocols.

In another preferred embodiment of the anchoring component comprises at least one thiol function, which allows for the attachment to metal clusters, such as, for example, gold nanoparticles. However, another function which is known to bind to a desired metal cluster or nanoparticle can also be used. A compound which has a carboxylate group or a phosphate group in one moiety and a thiol group in the other moiety is preferably used. A preferred example for an anchoring component comprising thiol functions is an α-dihydrolipoic acid residue (or a 6,8-dithioctic acid residue, TA). Depending on the nature of the surface of the metal cluster or nanoparticle to be used, i.e. either depending on the nature of the metal surface itself, or depending on the nature of a first activation or coordination layer, a skilled person will know which anchoring compound should to use to form an anchoring link in the sense of the invention.

Optionally, the anchoring component is linked to the transition metal complex by a suitable spacer component or moiety, such as, for example, an aliphatic hydrocarbon chain having at least one suitable functional group(s) for linking, such as, for example, a pentyl moiety having an amino group, or the like.

The anchoring component is directly linked to the transition metal complex. Preferably, the anchoring component is linked to the transition metal complex by a covalent bond, which is formed between an atom of the anchoring component and the ligand molecule of the transition metal complex. The anchoring component can also be linked to the transition metal complex by a coordination bond, which is formed between an atom of the anchoring component and the central transition metal atom of the transition metal complex. Especially preferred, the multidentate ligand of the transition metal complex comprises the anchoring component.

In a preferred embodiment of the probe compound of the present invention, the transition metal complex comprises a nitrotriacetic acid Co(II) complex. It was found that the presence of this transition metal complex allows for the most versatile adaptation of the probe compound for the identification of the reactive interaction with any small molecule or substrate tested so far. A preferred transition metal complex comprising an anchoring component is provided by using the compound N_{α},N_{α}-bis-(carboxymethyl)-L-lysine, or (S)—N-(5-amino-1-carboxypentyl)-imino-diacetic acid, respectively, as a ligand molecule for forming a cobalt(II) complex. In the so-formed cobalt complex, the aminopentyl residue, which is attached to one of the acetic acid groups of the ligand molecule, constitutes an anchoring component or a spacer moiety for attaching another anchoring component. The amine function of this anchoring component itself can be readily used to attach the transition metal complex, and thus the whole probe compound, to another component or molecule, such as, for example, a nanoparticle. Moreover, the amine function of this anchoring component can also be used to further attach a polymeric chain, such as, for example, a poly A chain, which can be used to attach the transition metal complex, and thus the whole probe compound, to a solid surface, such as, for example, a glass slide, in order to advantageously manufacture an array comprising corresponding probe compounds.

Thus, the term “probe compound” indicates a compound or molecule that can be immobilised on a supporting base by the anchoring component.

Preferably, the indicator component comprises a fluorescence dye, further preferred a fluorescent azo compound or a cyanine compound, examples of which are known in the art under the names of “Cy3” or “Cy5” or the like. By using this well-established dyes, it is possible to use the corresponding hardware available commercially, such as, for example, wave-length optimised reader apparatuses, corresponding software solutions, and the like. However, a person skilled in the art will understand that the present invention can easily be adopted to other indicator components and/or detection systems, if required. A person skilled in the art will also know how to carry out such adaptation.

Preferably, the test component comprises a known substrate, a metabolite, a pseudo-substrate, or an inhibitor of an enzyme. Further preferred, the test component comprises a molecule identified as a substrate of at least one metabolic reaction in the Kyoto Encyclopedia of Genes and Genomes (KEGG Database), the University of Minnesota Biocatalysis and Biodegration Database (UM-BBD), PubMed, or the like. Further preferred, the test component comprises a compound selected from the group listed in Table 1, 2 and 3, respectively.

By using such test components, it is possible to prepare probe compounds according to the invention which collectively form most of the central metabolic networks of cellular systems. However, the test component may also comprise additional metabolites characteristic of microbial metabolic activities not yet assigned or included in these databases, as well as

In a specially preferred embodiment of the probe compound of the present invention, the transition metal complex is represented by the N_{α},N_{α}-bis-(carboxymethyl)-L-lysine cobalt(II) complex, which may further comprise an anchoring component comprising a poly A chain or a 6,8-dithioctic acid (TA) linked to the free amine function of the ligand molecule. In this embodiment, an indicator component comprises a Cy3 fluorescent dye, which comprises a histidine moiety (His-tag) for linking to the transition metal complex and a aminobutyric acid moiety as an optional linker moiety for linking to the test component. The test component may comprise any enzymatic substrate or other suitable small organic molecule, for example, one of the substrates listed in Table 1. The test component is linked to the indicator component via the optional aminobutyric acid linker moiety and further comprises a histidine moiety (His-tag) for linking to the transition metal complex. Preferably, the link between the test component and the indicator component further comprises a quaternary amine function. By choosing the appropriate position for introducing the histidine moiety, the involvement of one substrate in different metabolic pathways can be detected by the probe compound. Preferred positions for binding the histidine moiety are shown in Table 2. The so-formed reactive component is linked to the transition metal complex by two coordination bonds, one of which comprises the His-tag linked to the test component, while the other comprises the His-tag of the indicator component. It was surprisingly found that, if the reactive component is linked to the transition metal complex, the characteristic fluorescence activity of the indicator component is no longer observed. In other words, the probe compound remains silent with respect to the characteristic fluorescence signal. This would result in a dark spot in an assay or array position. When the probe compound is brought into contact with an enzyme having a function specific to the test component or substrate comprised in the probe compound, the test component or substrate is metabolised. It was surprisingly found that this enzymatic reaction has two effects. First, the characteristic fluorescence signal of the indicator component is observed again, and second, the enzyme remains bound to the transition metal compound. Thus, the probe compound allows for the unambiguous detection of the specific enzymatic reaction by fluorescence detection. Moreover, the probe compound allows for an immobilisation of the substrate-specific enzyme, which can be advantageously used to isolate this enzyme from a sample.

In case of a probe compound of the invention comprising a Cy3 dye, it is observed that the fluorescence dye is quenched, i.e. does no longer emit its characteristic fluorescence signal, if the reactive component is linked to the transition metal complex to form the probe compound of the invention. In any other form, the dye emits its characteristic fluorescence signal and cannot be used to detect productive enzymatic reactions. Only when a productive enzymatic reaction occurs between an enzyme and the probe compound of the invention, the dye is released and the fluorescence is observed. This behaviour of the probe compound of the invention is the major difference to standard systems such as the labelling of proteins with a dye or DNA with a dye to produce protein arrays or DNA arrays, respectively, because the dye is permanently fluorescent in those cases.

The probe compound according to the above-discussed preferred embodiment of the invention can be illustrated by the following general formula (2):

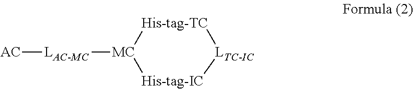

wherein AC represents an optional anchoring component, preferably poly A or TA, MC represents the N_{α},N_{α}-bis-(carboxymethyl)-L-lysine cobalt(II) complex, TC represents the test component, IC represents the fluorescence dye Cy3, His-tag represents a histidine moiety as described above, and L_{AC-MC} and L_{TC-IC} each represent optional linker moieties between the respective components indicated by the subscripts. Preferably, the L_{TC-IC} linker moiety comprises a quartenary amine function.

Although the exact mechanisms involved in these reactions are not completely understood yet, it is believed that the linking of the reactive component to the transition metal complex influences the indicator component in such a manner that its electronic structure necessary for its characteristic fluorescence signal is disturbed. The reaction of the enzyme with the test component or substrate is believed to alter or break down the reactive component, which influences the binding properties of the reactive component or its respective metabolised products, respectively, to the transition metal complex. Thereby, the adverse effect on the indicator component is no longer present so that the indicator component can again exhibit its characteristic fluorescence signal. Moreover, this enzymatic alteration or break down of the reactive component may also create new binding sites at the reactive component and/or the transition metal complex, which result in the binding of the enzyme.

The present invention also provides a method for preparing the probe compound. The method for preparing the probe compound of the invention comprises the following steps:

a) Preparing a transition metal complex by reacting a suitable salt of a transition metal with a desired ligand molecule, optionally comprising an anchoring component. Herein, the appropriate conditions of solvent, pH, temperature and the like can be chosen according to known procedures. If necessary, the thus-obtained transition metal complex can be further purified by standard procedures, such as, for example, filtration, re-crystallisation, or the like. In a preferred embodiment, N_{α},N_{α}-bis-(carboxymethyl)-L-lysine is reacted with cobalt(II) chloride to form the corresponding complex.
b) If an anchoring component is desired, it may also be incorporated at this stage. For example, in a preferred embodiment, the above cobalt(II) complex of the N_{α},N_{α}-bis-(carboxymethyl)-L-lysine ligand may be reacted with a suitably activated poly A chain or with 6,8-dithioctic acid, or the like.
c) In a separate reaction, a substrate molecule to be incorporated in the test component is coupled to an indicator component, optionally including a linker moiety. If necessary, either one or both of the test component and indicator component is previously transferred into an activated form suitable for coupling, according to standard techniques. Herein, the appropriate conditions of solvent, pH, temperature and the like can be chosen according to known procedures. For example, a Cy3 dye available as its Cy3-NHS-ester may be reacted with a 4-amino-3-butyric acid linker to allow for linking with the test component. Preferably, a linker moiety between the test component and the indicator component comprises a quaternary amine function (a quartenary ammonium nitrogen atom). If necessary, the thus-obtained reactive component can be further purified by standard procedures, such as, for example, filtration, re-crystallisation, or the like.
d) Optionally, the test component and/or the indicator component is functionalised with a moiety suitable for binding to the transition metal complex, either before or after formation of the reactive component. Such functionalisation is especially advantageous for indicator components or test components, which do not readily form coordination bonds. In a preferred embodiment, the test component and/or the indicator component is functionalised with a histidine molecule (sometimes referred to as a “His-tag” in the following). A His-tag has the additional advantage to ensure an identical binding property of all possible test components (substrates) and/or indicator components (dyes) to the transition metal complex.
e) In a subsequent step, the so-prepared reactive component comprising the test component and the indicator component is linked to the transition metal complex to form the probe compound according to the present invention. Herein, the appropriate conditions of solvent, pH, temperature and the like can be chosen according to known procedures. If necessary, the thus-obtained probe compound can be further purified by standard procedures, such as, for example, filtration, re-crystallisation, or the like.

The method comprises the steps a), c) and e), and the optional steps b) and/or d), if desired. The optional steps b) and d) can be carried out at any stage of the method. For example, step d) may be carried out before step c), or subsequent to step b), and step b) may be carried out before step a), subsequent to step a), or subsequent to step e), respectively. In a preferred embodiment, the individual steps are carried out in the order of steps a), b), d), c, and e). Alternatively, the steps may be carried out in the order of steps b), a), c), d), and e), or in the order of steps a), d), c), e), and b). A person skilled in the art will know which order and conditions are most suitable for the respective task.

Preferably, the step for linking the indicator component to the test component and/or the step for linking the histidine residue to the test component involves an activation of the test component by specific halogenation with iodine (I). For example, a selected position (atom or group) of the substrate or metabolite component comprised in the test component is selectively halogenated with iodine. This can be done by standard chemical reactions or enzymatically. The halogenated position allows for substitution reactions replacing the iodine atom by the corresponding component or residue.

The present invention also provides an array for detecting enzymes comprising a plurality of different probe compounds according to the invention. The array comprises a plurality of different probe compounds according to the invention (sometimes referred to as library of probe compounds in the following), i.e. probe compounds according to the invention which differ at least with respect to the test component comprised therein. Preferably, the probe compounds only differ with respect to the test component comprised therein. A plurality of different probe compounds can be prepared using automatic procedures, for example, parallel synthesis protocols and apparatuses, or the like. The term “comprising a plurality of different probe compounds” means at least two different probe compounds, and may comprise up to several thousands different probe compounds. For example, apparatuses and protocols, which are known and commercially available at the moment, allow the standard production of arrays based on glass slides wherein from 5000 to 15000 different probe compounds may be deposited on a single glass slide by micro-spotting techniques, or the like. In a preferred embodiment, about 2500 different probe compounds are included in the array (cf. example). Alternatively, the array may be constructed using a plate providing separate compartments or wells, such as, for example, a micro-titre plate, which is commercially available with, e.g., 384 wells, or different numbers of wells. A complete array may comprise one or more slides or plates depending on the actual number of probe compounds included in the array, as well as on the density available on the respective medium. Preferably, an array comprises more than one copy of a library of probe compounds on one or more supports. For example, a library or sub-library of probe compounds may be provided in duplicate or triplicate on one support or slide.

In one embodiment of the array, the plurality of probe compounds is attached to a planar surface of a suitable support or carrier. A support may be any support used in the art, preferred examples of which are glass surfaces, preferably of glass slides, surfaces of polymeric materials, such as polyacetate surfaces, or the like, or surfaces of cellulose or paper materials, or the like. In order to be attached to a solid surface, such as, for example, the glass surface of a glass slide, the probe compounds are provided with a suitable anchoring component. A preferred anchoring component known from the art is a poly A chain or tail, which can be readily attached onto a glass surface or the like following standard protocols using irradiation by UV light. Other suitable solid surfaces and corresponding anchoring components as well as protocols for their use are known in the art, and a person skilled in the art will be able to select an appropriate combination. For example, when using polymeric supports, it is advantageous for the bonds between the probe molecules and the polymeric support to be chemical bonds, especially covalent chemical bonds, which allow a long-lasting, stable bond between the probe molecules and the polymeric support.

In the case of using a well plate, the probe compounds do not necessarily require an anchoring component because they can be held in the respective wells without attachment to the wall. Preferably, probe compounds are attached to the walls of a well of a well plate. Therefor, all methods known in the art may be used.

The array of the invention (which is sometimes referred to as “reactome array”) can be used for the simultaneous detection of reactive interactions between the probe compounds and all analyte molecules (enzymes) from a sample. In particular, the array provides a solution to the problem of the identification of metabolites and the enzymes involved in their transformation. The array provides a fast and reliable way to detect all metabolic pathways active in an organism or community, and may be used advantageously for an activity-based, annotation-independent procedure for the global assessment of cellular responses.

The invention also provides a method for producing an array according to the invention. An array may be produced using well plates, such as commercially micro-titre plates, wherein each probe compound of the plurality of different probe compounds according to the invention is filled into an individual well, together with appropriate amounts of solvent, cofactors, cations, supplements, or the like, which are known or predicted to be required for the expected enzyme reaction. The filling of the wells may be carried out automatically, e.g. by a suitable robotic apparatus, or the like. Alternatively, the array is preferably produced using different probe compounds, which all comprise the same anchoring component. The different probe compounds having an anchoring component are then arranged onto an appropriate support surface, e.g. the surface of a glass slide, and subsequently bound thereto with the anchoring component. The arranging and binding of the different probe components may be carried out automatically, e.g. using a suitable robotic apparatus, or the like, and following established procedures and protocols. In a preferred embodiment, different probe compounds comprising a poly A chain as an anchoring component are spotted onto a glass slide by using a robotic apparatus, and subsequent fixation is achieved by cross-linking the poly A tails according to an established protocol using UV radiation. However, several other methods for arranging and fixing the different probe molecules onto a suitable support surface are known in the art. Using the method of the invention allows for a versatile, fast and reproducible production of arrays according to the invention.

Moreover, the invention also provides an isolation means comprising a nanoparticle and a probe compound according to the invention. A probe compound is preferably provided with a suitable anchoring component and attached to a nanoparticle. The term “nanoparticle” means a particle having a maximum dimension of less than 500 nm, preferably of less than 300 nm, and most preferred of about 100 nm. A minimum dimension is about 10 nm, preferably about 50 nm. A maximum or minimum dimension of a nanoparticle refers to the diameter in the case of a spherical nanoparticle. Examples for suitable nanoparticles are known in the art, as well as methods for producing the same, or anchoring components for linking the probe compound to the same. The probe compound may be linked to any nanoparticle known in the art, preferably a magnetic nanoparticle. Preferably, a nanoparticle comprises one or more metallic elements, including the transition metal elements. Preferred examples of nanoparticles comprise metallic elements, either pure metallic elements, such as, for example gold nanoparticles, or alloys comprising different metallic elements, or oxides of metallic elements, such as, for example, iron oxides, or the like. Preferred oxidic nanoparticles may comprise silicon, e.g. in form of silicon oxide. Moreover, preferred nanoparticles may also have a layered structure, e.g. an oxidic core coated by a metallic layer, such as a gold-coated silicon oxide nanoparticle, or a metallic core coated by an oxidic layer, such as a cobalt core coated with an iron oxide layer. For example, the synthesis and application of suitable magnetic gold nanoparticles is described by Abad et al. in J. Am. Chem. Soc. 127, 5689 (2005). Preferably, a nanoparticle has a magnetic property. A preferred anchoring component for linking a probe molecule to a magnetic gold nanoparticle is 6,8-dithioctic acid or α-dihydrolipoic acid, respectively, which advantageously may be linked to a transition metal complex comprising the cobalt(II) complex of N_{α},N_{α}-bis-(carboxymethyl)-L-lysine (ANTA-Co (II)) by forming an amide bond between the carboxylic acid function of the 6,8-dithioctic acid and the amine function of the ligand molecule. The isolation means according to the invention allows for the substrate specific interaction and binding of an enzyme which can then be isolated by means of filtration, gravitation force (centrifugation), an external magnetic force in case of a magnetic nanoparticle, or the like. It was found that, owing to the substrate specific binding of an enzyme by the probe compound, the isolation means of the invention allows for the directed isolation of an enzyme because of its substrate specificity.

The invention also provides a method for producing a isolation means according to the invention. In this method, a probe compound comprising a suitable anchoring component is prepared according to the method of producing a probe compound according to the invention, and subsequently attached to a suitable nanoparticle prepared according to known methods. Preferably, the probe compound is attached to a magnetic nanoparticle. The method according to the invention provides an easy access to an isolation means having specific binding sites for an enzyme.

Moreover, the invention also provides a method for detecting enzymes in a substrate specific manner, using the probe compound according to the invention or the array according to the invention. An array comprising a plurality of different probe compounds is prepared according to the method of the invention. The array can preferably comprise more than one copy of the respective library of probe compounds, either by providing the library on one support or slide in duplicate or triplicate or in more copies, or by providing more than one support or slide comprising identical sets or libraries of probe compounds. The array is then brought in contact with a sample comprising the analyte molecules, i.e. the enzymes whose substrate specific activity is to be tested. This step is also referred to as incubation with a sample. A sample in the context of the method includes any kind of solution of analyte molecules (enzymes) that can enter into an enzymatic reaction with the probe compounds on the array. These include especially biological samples obtained from the lysis of cells of bacteria, archeae, or higher organisms, but also from biological fluids such as blood, serum, secretions, lymph, dialysate, liquor, sap, body fluid from insects, worms, maggots, etc. Also included is extraction from natural sources such as biopsies, animal and plant organs or parts, cell, insect, worm, bacteria, microbe and parasitic matter as well as supernatants of cell cultures and of bacterial, microbial or parasitic cultures. A sample may also be a chemical-synthetic sample containing, for example, synthetic proteins, or the like. After incubation is complete, the sample is removed from the array. In order to obtain a complete removal of the sample, the array may be washed one or more times. Then, the array is analysed for the presence of the signal characteristic for the indicator component. Preferably, a fluorescence signal is read out and processed by a suitable apparatus, which is available in the art. All protocols and procedures known in the art may be used, including automatic processing by robotic apparatuses and the like. In a preferred embodiment, using an array comprising probe compounds comprising a Cy3 fluorescence dye, the fluorescence signal is preferably measured using a laser scanning system.

The method of the invention using the array of the invention allows to assess the complete reactome of an organism or community in one step. That is, all substrate specific enzymatic reactions which are performed within the metabolic pathways necessary for the individual life form(s) is readily accessible. This advantageously allows for the accurate reconstruction of metabolic pathways.

Moreover, the invention also provides a method for isolating enzymes using the isolation means according to the invention. According to the method, the nanoparticles are used to isolate enzymes which are bound to the isolation means by the probe molecules after the substrate specific enzymatic reaction. The reaction can be carried out under the same conditions as described above for the array of the invention, including all processes and protocols that are known in the art. Preferably, the isolation means is brought into contact with a sample comprising analyte molecules (enzymes) that can enter into an enzymatic reaction with the probe compounds on the isolation means. The isolation means carrying the enzymes are then isolated by magnetic means or by filtration. The isolated enzymes can then be analysed using standard techniques, such as, for example, sequence analysis, mass spectrometry, or the like. Identification of enzymes whose function is verified by the specific reaction with the probe compound of the invention allows for the detection of metabolic pathways as well as for the correct annotation of unsequenced genes and/or unknown proteins or enzymes. The method can be used to complete the metabolic pathway map of all life forms, as well as specialised parts thereof. The method can also be used to reconstruct the global metabolism of complete communities living in distinct natural (microbial) communities.

Moreover, the method of the invention can be advantageously used in the search for new drugs, in particular in screening for potential new targets: here the guiding principle is selective toxicity, so the search is for molecular targets in the target organism that do not exist in a human. The method of the invention is also useful for the identification of drugs that are specific for certain pathogens, so that treatment of an infected patient would not result in the elimination of a major part of the body flora, as is currently the case with common broad spectrum antibiotics, and all the negative consequences of this that ensue. Using the array to obtain reactome profiles of the representatives of the major phyla of life allows their comparison and identification of individual reactions/enzymes that are specific for each branch or clusters of branches. Some of these phylum-specific functions will be known already, but others may be new: such metabolic reactions/enzymes may then serve as newly-discovered targets for drug screening. For example, the comparison of the human reactome with the composite reactome of bacteria and archaea may identify new targets for broad spectrum antibiotics, or the comparison of the reactomes of the various bacterial phyla may reveal potential targets specific for individual phyla, such as those to which, e.g., Neissseria, Pseudomonas, Mycobacterium, Vibrio, Staph, Strep, Pneumo, coliforms belong, providing a route to more specific, narrow spectrum antibiotics. Moreover, the comparison of reactomes of photosynthetic organisms may identify new targets for herbicides specific for, e.g., moss, or algae, or monocots, or dicots etc. that would allow the development of new generation anti-moss treatments that would not affect other plants, anti-algal treatments not active against other plants, etc., etc. Moreover, the comparison of the reactomes of insect vectors of disease (mosquito, black fly, etc) with those of other insects, humans etc. could identify vector-specific targets.

In other words, the present invention provides a versatile tool for obtaining information on the reactomes of all major branches of the tree of life which can be advantageously used in future drug development. Through the selective inhibition of pathogens, there become available a lot of applications involving the selective inhibition of specific microbial populations, either because they are known to be problematic, or simply to test experimentally their contribution to a particular process (e.g. the role of a particular GI tract organism in a particular GI physiological role, like folate production, the role of a particular rhizosphere organism in protection from fungal infections, etc.). Thus the array provides the means of identifying phylum-specific metabolic targets for inhibitors that, in turn, can be used to inhibit specifically those phyla in microbial communities to assess their role in a particular process.

The use of the reactome array of the invention for identifying new targets can be applied from human medicine to agriculture to dental medicine to shipping (anti-algal compounds to prevent algal fouling; anti-sulphate reducer compounds to prevent corrosion of steel) to construction (e.g. anti-sulphate reducer compounds to prevent corrosion of steel), etc., and to research tools (agonists/antagonists for all manner of selected life forms).

The present invention provides a procedure to measure on an array, enzymatic activities of cells against many of the standard substrates and metabolites that characterize life, plus other substrates of interest. Because of the chemical design of the substrates of the array, the array provides the identity of the reaction, the reaction products and, in a subsequent step, the enzyme itself. It therefore links a substrate/metabolite with its cognate enzyme. The reactome array thus forges a thus far missing link between metabolome and genome. Since many of the metabolites on the array are connected in pathways, it is also possible to reconstruct the metabolic network operating in any organism without any prior genomic information.

The use of the array to investigate the reactome of a microbial community is particularly interesting. The array represents a new possibility to have a metabolic overview of an entire community, without perturbing it in any way prior to preparation of the community lysate for analysis. And, in cases where it is difficult to obtain sufficient biomass for direct analysis, it is even possible to obtain a small molecule microarray analysis of a metagenomic library of a DNA sample of a community or enrichment and thereby obtain a detailed overview of the global metabolism of the sampled community. The reactome analysis described here uncovered new metabolic activities in organisms and communities (46 new functions in P. putida KT2440 and five in metagenomic communities, including a novel hydrogenase; cf. Table 4 and FIGS. 6 and 7, SEQ ID NOS. 13 to 16, 21 to 24, 29 to 32, 38 to 43, and 50 to 54), which not only provide interesting opportunities for new lines of investigation, but also revealed new metabolic components of niche specificity and predominant microbial pathways shaping the overall metabolism of the individual habitats. Especially, the hydrogenase (cf. Table 4 and FIG. 7, SEQ ID NOS. 50 to 54), which is a rather small enzyme, may be of interest in the field of energy production. Moreover, a novel reBr halogenase/dehalogenase, i.e. a multifunctional a/β-hydrolase, was mined from a metagenome library of a microbial community in seawater contamined with petroleum hydrocarbons, with a novel hydrolytic phenotype, namely the cleavage of both ‘common’ p-nitrophenyl (pNP) esters and haloalkanoates, and weak activity towards haloalkanes (paper in preparation, SEQ. ID. NOS. 57 to 59). This halogenase/dehalogenase enzyme was found to be useful for the introduction of iodine into the test component in the syntheses of the probe compounds of the present invention.

Since a physico-chemical analysis of habitats is rather selective in terms of the parameters measured, detection limits defined by the instruments used, and usually does not discriminate between bioavailable and non-bioavailable levels, whereas the array scores most of the metabolic potential of the cell or community in relation to the prevailing conditions and bioavailable fraction of compounds, another application of the array is the habitat characterization by metabolic profiling. This type of application can also be used in diagnosis of diseases/intake of drugs/toxic substances that influence metabolic activities of the microbial flora, through reactome analysis of faecal/skin biota, forensic analyses of diverse types (e.g. groundwater pollution), prospecting (e.g. for natural gas seeps that indicate underlying reservoirs), detection of manufacturing sites of illegal substances, etc. Indeed, the design of custom arrays for use with particular organisms or communities will entrain a diverse spectrum of applications relating to enzyme activity profiles, including the phenotyping of organisms (microbes, plants, higher animals and humans), populations, mutant libraries and transgene libraries, the direct diagnosis of diseases and quality control in food industries, to cite some.

The present invention will be illustrated in more detail in the following examples, but it is not restricted to the special embodiments exemplified in these examples.

Commonly used chemical and molecular-biological working methods are not described in detail here, but they can be referred to in, for example, Houben-Weyl, Methods of Organic Synthesis.

### EXAMPLES

### Example 1

### General Techniques

Unless specified otherwise, reactions were carried out with dry solvents freshly purified by passage through a column of activated alumina (A-2) and supported copper redox catalyst (Q-5 reactant). All other reagents were purified according to standard literature methods or used as obtained from commercial sources.

NMR spectra of all compounds were recorded at 600, 500, 400, or 300 MHz, using Varian I-600, Varian I-500, Varian M-400, Varian M-300, and Bruker Biospin 300 instruments. ¹H NMR chemical shifts were reported relative to residual CHCl₃ (7.26 ppm). ¹³C NMR data were recorded at 125, 100, or 75 MHz, using Varian I-500, Varian M-400, or Bruker Biospin 300 MHz instruments, respectively. ¹³C NMR chemical shifts are reported relative to the central line of CDCl₃ (77.0 ppm). ⁵⁹Co NMR measurements were carried out at room temperature with Bruker ASX-200 (B₀=4.7 T, Larmor frequency v₀=48.1 and 52.9 MHz in ⁵⁹Co resonance, Bruker MSL-300 (B₀=7.1 T, v₀=71.2 and 79.4 MHz), and Bruker Avance DSX-500 (B₀=11.7 T, v₀=120.4 and 132.3 MHz) spectrometers. Single-pulse MAS spectra were obtained by using a Bruker MAS probe with a cylindrical 4-mm o.d. rotor. When necessary, continuous-wave proton decoupling with a radiofrequency (RF) field of 50 kHz was applied during acquisition. Spinning frequencies vᵣ up to 17 kHz were utilized. A short pulse length of 1 μs corresponding to a nonselective π/12 pulse determined using an aqueous or DMSO solution of small molecules (SMs) was employed. Recycle times were 1 and 90 s in ⁵⁹Co. The baseline distortions resulting from the spectrometer dead time (5-10 μs) were removed computationally using a polynomial baseline correction routine. The dead-time problem was then overcome by Fourier transformation of the NMR signal, starting at the top of the first rotational echo.

Molecular masses were analyzed at the SIDI Core Facility of the Autonomous University of Madrid. For each experiment, a magnetic high resolution mass spectrometer (8000 v acceleration) was used, with ionization source FAB (LSIMS—liquid secondary ion mass spectrometry with Cs ions) using m-nitrobenzoic alcohol (m-NBA) as matrix. The samples (0.5-1.2 g) were dissolved in acetone, methanol or DMSO, depending of the solubility of the SMs-Cy3. Microanalyses were performed by the SIDI Core Facility of the Autonomous University of Madrid, and are quoted to the nearest 0.1% for all elements, except for hydrogen, which is quoted to the nearest 0.05%. Reported atomic percentages are within the error limits of ±0.3%.

For N-terminal amino acid sequencing, polyacrylamide gel electrophoresis under denaturing conditions (SDS-PAGE: 10%, v/v) was performed according to Laemmli (U.K. Laemmli, Nature 227, 680 (1970)), using a Mini-PROTEAN cell apparatus (Bio-Rad), and protein bands were blotted to a polyvinylidene difluoride (PVDF) membrane. The PVDF membrane was stained with Coomassie Brilliant Blue R-250, after which the bands of the proteins were cut out and processed for N-terminal amino acid sequencing. The peptide sequences were initially scored against blastp nr to identify the best hits among full-length proteins, and then converted into coding sequences and screened for potential protein encoding genes (PEGs) via tblastn and tblastx search (F. Stephen et al., Nucleic Acids Res 25, 3389 (1997)) against the comprehensive non-redundant database sourced from the nucleotide (nr/nt) collection, reference genomic sequences (refseq_genomic), whole genome shotgun reads (wgs) and environmental samples (env_nt) databases. This was chosen empirically to increase the number of matching potentially coding elements. Based on the best BLAST hits, degenerate oligos were designed and used to amplify full length ORFs from the metagenome libraries.

### Example 2

### Bacterial Strains, Culture, and Growth Conditions

E. coli DH5F′ was used as a recipient for pGEMT plasmid (Promega) constructs containing cloned PCR fragments of P. putida KT2440 encoding hypothetical proteins and metagenomic proteins. E. coli TOP10 (Invitrogen) was used as a recipient for pCCFOS vector (EPICENTRE) constructs. E. coli cultures were grown in Luria-Bertani (LB) medium and incubated at 37° C. on an orbital platform operating at 200 rpm. When required, cultures were supplemented with the following antibiotics: ampicillin (100 μg/ml), nalidixic acid (10 μg/ml) and chloramphenicol (12.5 μg/ml). P. putida KT2440 was grown in M9 minimal medium with 15 mM succinate as carbon source in 100-ml flasks shaken at 30° C. and 150 rpm from an initial turbidity at 600 nm of 0.02 to a final value of 0.7±0.05. Samples (3 ml) were removed, the cells harvested by centrifugation at 4° C., and the cell pellets were washed with 20 mM Hepes pH 7.0 before storing at −20° C. until use.

### Example 3

### General Reactome Strategy

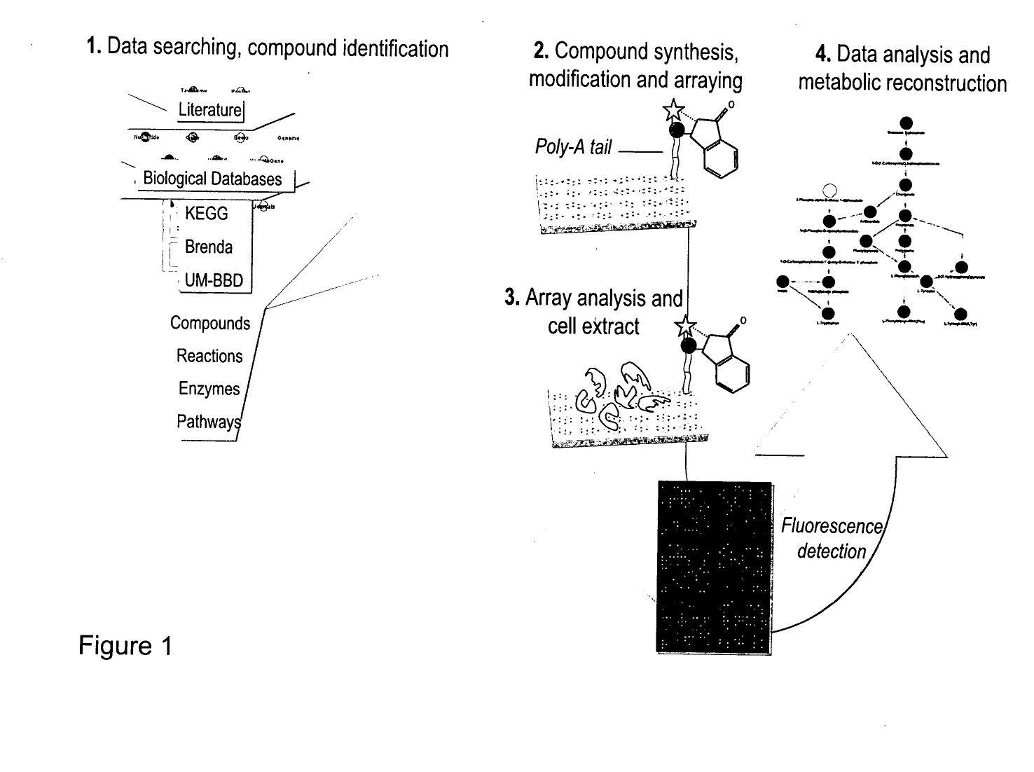

The general reactome strategy comprises five stages for array construction and protein-SMs transformation detection as follows (cf. FIGS. 1 to 3).

### [1] Data Searching and Compound Identification

Initially, an extensive data mining effort, focused mainly on the Kyoto Encyclopedia of Genes and Genomes (KEGG Database: http://www.genome.ad.jp/kegg/), the University of Minnesota Biocatalysis and Biodegradation Database (UM-BBD: http://umbbd.msi.umn.edu/) and PubMed (http://www.ncbi.nlm.nih.gov/sites/entrez), was undertaken to produce a list of compounds to be synthesized that are substrates of one or more metabolic reactions and that collectively form most of the central metabolic networks of cellular systems. Additional metabolites characteristic of microbial metabolic activities were also identified for synthesis.

### [2] Compound Synthesis, Modification and Arraying

A library of 2483 identified SMs was synthesized using the strategies specified in Table 1. The purity of each SM was confirmed by NMR and molecular mass. Individual SMs were coupled to the Cy3 fluorescent dye and subsequently combined on specific positions to a nitrotriacetic-Co(II) complex containing a terminal poly A-tail (Table 2). Importantly, during synthesis, the Cy3 dye is attached to the molecule at specific positions that allow control of the reaction product formed, through creating for each molecule different Cy3-variants that collectively serve as substrates for all possible reactions described in KEGG; the array thus assays multiple distinct reactions of the same metabolite. The resulting derivatives were dissolved at different concentrations from 0.5 to 100 nM (six concentrations) in dimethyl sulfoxide (DMSO) and stored at −70° C. until use in 384-well microtiter plates. Each well also contained cofactors, cations and supplements known or predicted to be required for efficient transformations. The 2483 SMs included in this study, together with the position of modification with Cy3 are given in Tables 1 and 2. These procedures were also used to synthesize Cy3-modified X-Gal (obtained from Boehringer Mannheim), which was used as substrate for the β-galactosidase of E. coli (provided also by Boehringer Mannheim). Individual SMs were subsequently spotted onto Corning UltraGAPS glass slides in a spatially addressable manner, by means of a MicroGridII spotting device from Biorobotics operating at 20° C. and 50% relative humidity, and subsequently immobilized by standard UV cross-linking.

### [3] Array Analysis and Cell Extract

60 μl quantities of cell lysates of microbial cultures, or libraries of metagenomic clones of microbial communities, diluted in PBS buffer to a final protein concentration of 0.1 mg/ml, were layered on the array, which was subsequently incubated at room temperature for 30 min.

### [4] Data Analysis and Metabolic Reconstruction

Arrays were scanned for fluorescence with a GenePix 4000B scanner (Axon Instruments) operating at 532 nm at 10 resolution with 100% laser power, and images (spot and background intensities were quantified in triplicate or more) were analyzed using the GenePix v5.1 software (Axon Instruments). Reconstruction of metabolic maps were carried out with GraphViz and SOI Linux software.

[5] Protein Identification with Gold Beads

Proteins reacting with specific metabolites were identified by incubating a protein extract with metabolite-coated gold nano-particles, followed by protein sequencing of the captured proteins, reverse translation of the sequences into gene sequences, cloning of the corresponding genes, hyper-expression of the genes, and purification and characterization of the corresponding proteins.

### Example 4

### General Procedure for the Synthesis of Probe Compounds

The general reaction strategy showing the successive steps for the construction of Cy3 modified metabolites and their integration into probe compounds is described below.

### 1. Preparation of nitrilotriacetic-Co(II) complexes

The amino-nitrilotriacetic-Co(II) complex was formed by reaction of NR,NR-bis(carboxymethyl)-L-lysine hydrate (ANTA, Fluka) with an excess of cobalt(II) chloride (Sigma) in 20 mM HEPES in aqueous solution. (36). Excess cobalt was precipitated by increasing the pH to 10, and the precipitate was removed by filtration through a 0.2 μm membrane (PTFE, Amicon).

### 2. Incorporation of poly(A) tails in nitrilotriacetic-Co(II) complexes (optional)

Activation of the phosphate groups of the poly(A) tails (Sigma Genosys, average molecular weight: 100 kDa) and subsequent amidation with the Co(II) complex was performed by overnight incubation with 3 mM N-hydroxysuccinimide (NHS, Fluka) and 3 mM 1-ethyl-3-[3¢-(dimethylamino)propyl]-carbodiimide (EDC, Sigma) in 20 mM HEPES buffer (pH 7.5).

### 3. Generation of double activated Cy3 dye

Cyanine dye was linked via a histidine tag and a flexible linker through which the dye is linked to the Co(II) complex and the metabolite, respectively. Briefly, a histidine molecule was firstly incorporated to the Cy3 dye by enzymatic acidolysis of Cy3 NHS (a succinimidyl ester, GE Healthcare) with histidine and immobilized Lewatit lipase EL1 (37) from a cow rumen metagenome (37.39 mol % incorporation of histidine in 24 h, at a ratio 1 histidine:4 NHS ester). 3-Methyl-2-butanol was used as solvent with a water content of up to 3.2%, and the reaction was carried out at 50-55° C. High-performance preparative liquid chromatography was used to analyze and purify the products of the acidolysis reaction. Purified Cy3-His was dissolved in DMSO at a concentration up to 4 M and stored at −70° C. until use

In a second step, the Cy3-His was joined to a 4-amino-3-butyric acid linker. Briefly, the linker was dissolved in 0.1 M sodium borate buffer pH 8.5 and Cy3-His dissolved in a small amount of neutral water was added in aliquots until equimolar concentrations were reached. After incubation for 2 hours at room temperature, the labeled product was purified by reverse phase HPLC on a Chemcobond 5C18 ODS column (4.6×150 mm). Elution was carried out with a linear gradient of 6% to 50% acetonitrile in 50 mM ammonium formate, pH 7.0, over a period of 30 min at a flow rate 1.0 mL/min, with monitoring of the eluate at 550 nm. The yield was 46%. HRMS (MALDI) calculated for C₄₄H₅₅N₆O₁₁S₂ [M⁺] was 908.0906 and found was 908.0955. The Cy3-His-4-amino-3-butyric acid (N,N-dimethylamino) ethylester was synthesized by Lewatit lipase EL1-mediated esterification as described above. The final product was purified by reverse phase HPLC, as described above, except that the gradient was 6% to 80% acetonitrile. The yield was 0.1%. HRMS (MALDI) calculated for C₅₃H₆₄N₉O₁₃S₂ [M⁺] was 1099.2815 and found was 1099.2841.

### 4. His-tagged metabolite library synthesis

The primary synthetic challenge involved finding a reaction path from a functionalized core with the highest yield (>26-90% overall). The purified metabolites were characterized by NMR and HPLC and were found to be 90% pure; yields were >20%. A number of standard strategies were employed, and new ones developed for the synthesis of compound libraries synthesized on solid supports or by parallel synthesis using separate reaction vessels. The full spectrum of synthesis methods used in the present study (as well as NMR and high resolution mass spectra) will be available to the community on our web server (http://biology.bangor.ac.uk/people/staff/025123) and are briefly described in Table 1, together with metabolite synthesis strategies. In some cases, the metabolites were directly purified from pure cultures using preparative HPLC (Waters 2795 XE). The purity of each SM was confirmed by NMR and mass determination (see Table 1). SMs were reconstituted and diluted in PBS buffer, DMSO or a mixture of both, accordingly to their solubility properties, and stored in 384-well microtiter plates at −70° C. until used. Metabolites were further functionalized via incorporation of a histidine tag at specific positions (SM-His; see Tables 1 to 3) using solid-solid and solid-liquid phase synthesis.

General synthetic methods to incorporate His-tags to different metabolites (SM) are listed in the following.

| Metabolite | |
|---|---|
| characteristics | Synthetic method to incorporate His tags |
| OH-containing | Lipase (Thermomyces lanuginosus) |
| metabolites | esterification with histidine in 3-Methyl-2- |
| | butanol (Ferrer et al., Tetrahedron (2000) |
| | 56: 4053-4061) |
| NH₂-containing | Lipase (Candida antarctica) amidation with |
| metabolites | histidine in 3-methyl-2-butanol (Plou et |
| | al., Journal of Biotechnology (2002) 96: 55- |
| | 66) |
| Aliphatic | Enzymatic incorporation of OH— groups via a |
| metabolite | wide spectrum dioxygenase followed by |
| (linear) | esterification with lipase (Thermomyces |
| | lanuginosus) and histidine in 3-methyl-2- |
| | butanol. |
| Aliphatic | The metabolite is dissolved in |
| metabolite | trifluoroacetic acid (20 ml) and refluxed |
| (circular) | for two hours under nitrogen. The solvent is |
| | evaporated and the residue is extracted with |
| | ethyl acetate. The organic layer is washed |
| | with water, brine and dried over MgSO4. The |
| | hydroxylated compound is then esterified |
| | with histidine using Thermomyces lanuginosus |
| | lipase in 3-methyl-2-butanol. |
| Aromatic | The —OH group is incorporated as described |
| | by Callahan et al., Bioorganic and Medical |
| | Chemistry Letters 16, 3802 (2006) and the |
| | hydroxylated compound is then esterified |
| | with histidine using Thermomyces lanuginosus |
| | lipase in 3-methyl-2-butanol. In this case a |
| | double bond is lost during the synthesis. |

### 5. Generation of His-tagged metabolite-Cy3 library

Incorporation of the fluorescence dye in the metabolite was achieved by reaction of the SM-His molecules with activated Cy3-His-4-amino-3-butyric acid (N,N-dimethylamino) ethylester, using solid-solid and solid-liquid phase synthesis. The resulting library is composed of individual metabolites coupled at different positions to Cy3 dye molecules, both of which carry histidine tags. The coupling of Cy3 at different positions was designed to create the full spectrum of potential substrates needed to detect all possible catalytic reactions described in KEGG for a given core metabolite. Thus, each type of Cy3 substituent configuration determines the identity of the reaction and the reaction products. And, only when a reaction occurs at a specific position is the Cy3 dye released to give a fluorescent signal.

General synthetic methods to incorporate different His-tagged metabolites (SM) to the activated Cy3 are listed in the following.

| Metabolite | |
|---|---|
| characteristics | Synthetic method to incorporate His tags |
| Halogenated | The activated Cy3 is reacted with SM-His |
| metabolites | halide in the presence of a quaternary |
| | ammonium salt such as benzyltriethylammonium |
| | chloride using 50% aqueous sodium hydroxide as |
| | an acid-removing agent (Bull. Chem. Soc. Jpn., |
| | 54, 1879 (1981)). |
| Non halogenated | An halogenated moiety is incorporated via |
| aliphatic | treatment of the His-SM with CHCl3 + Fe(CO)5. |
| metabolite | Afterwards, the halogenated derivative is |
| (linear) | incorporated to the Cy3 as above |
| Non halogenated | Enzymatic C—C bond formation via a wide |
| aliphatic | spectrum aldolase. |
| metabolite | |
| (circular) | |
| Aromatic | Aromatic nucleophilic substitution reaction |
| | between His-SM and activated Cy3 in anhydrous |
| | dimethylsulfoxide as described by Médebielle |
| | (Tetrahedron Letters, 37, 5119-5122 (1996)) |

### 6. Incorporation of His-tagged metabolite-Cy3 derivatives to the poly(A)-nitrilotriacetic-Co(II) complex

The final step in the array development is the incubation of the histidine functionalized Cy3-SM with poly(A)-nitrilotriacetic-Co(II) complexes in 50 mM phosphate buffer, 50 mM NaCl, pH 7.5 for 1 hour at 25° C. When required, DMSO was added to increase substrate solubility. The resulting complexes were separated from unbound enzyme molecules by HPLC as described above. To ensure that each SM-Cy3 binds through both of its His residues, ⁵⁹NMR was performed, and only derivatives incorporating single SM-Cy3 molecules were purified, and stored in 384 microtiter plates at −70° C. until used. These molecules were used directly for the construction of the array.

### 7. Binding of His-tagged metabolite-Cy3 derivatives to gold nanoparticles (optional)

Au-6,8-dithioctic acid (TA) clusters were synthesized as described by Abad et al. (Abad et al. in J. Am. Chem. Soc. 127, 5689 (2005)) and used to create Au-TA-ANTA-Co(II)-SMs-Cy3 clusters. Briefly, the Au-TA clusters were linked to the ANTA-Co(II)-SMs-Cy3 (prepared as described above) by overnight amidation in a single step in the presence of 3 mM N-hydroxysuccinimide (NHS, Fluka) and 3 mM 1-ethyl-3-[3-(dimethylamino)propyl]carbodiimide (EDC, Sigma) in 20 mM HEPES buffer (pH 7.5). Further purification was carried out by ultrafiltration through low-adsorption hydrophilic 30000 NMWL cutoff membranes (regenerated cellulose, Amicon).

### Synthesis Example 1

The following Synthesis Example 1 provides a complete synthesis of a probe compound comprising 5-bromo-4-chloro-3-indoyl-beta-galactopyranoside (X-Gal) as test component (SM) and Cy3 as indicator component. Both the test component and the indicator component are linked to a transition metal complex comprising cobalt by one histidine linker moiety each. Test component and indicator component are linked by 4-amino-3-butyric acid as a linker moiety. The so-prepared probe compound is used for the analysis of the sensitivity of the reactome array.

### 1. Preparation of nitrilotriacetic-Co(II) complexes

The amino-nitrilotriacetic-Co(II) complex was formed by reaction of NR,NR-bis(carboxymethyl)-L-lysine hydrate (ANTA, Fluka) with an excess of cobalt(II) chloride (Sigma) in 20 mM HEPES in aqueous solution (J. M. Abad et al., J Am Chem Soc 127, 5689 (2005)). Excess cobalt was precipitated by increasing the pH to 10, and the precipitate was removed by filtration through a 0.2 μm membrane (PTFE, Amicon).

### 2. Incorporation of poly(A) tails in nitrilotriacetic-Co(II) complexes

Activation of the phosphate groups of the poly(A) tails (Sigma Genosys, average molecular weight: 100 kDa) and subsequent amidation with the Co(II) complex was performed by overnight incubation with 3 mM N-hydroxysuccinimide (NHS, Fluka) and 3 mM 1-ethyl-3-[3¢-(dimethylamino)propyl]-carbodiimide (EDC, Sigma) in 20 mM HEPES buffer (pH 7.5).

### 3. Generation of double activated Cy3 dye

Cyanine dye was linked via a histidine tag and a flexible linker through which the dye is linked to the Co(II) complex and the metabolite, respectively. Briefly, a histidine molecule was firstly incorporated to the Cy3 dye by enzymatic acidolysis of Cy3 NHS (a succinimidyl ester, GE Healthcare) with histidine and immobilized Lewatit lipase EL1 (37) from a cow rumen metagenome (37.39 mol % incorporation of histidine in 24 h, at a ratio 1 histidine:4 NHS ester). 3-Methyl-2-butanol was used as solvent with a water content of up to 3.2%, and the reaction was carried out at 50-55° C. High-performance preparative liquid chromatography was used to analyze and purify the products of the acidolysis reaction. Purified Cy3-His was dissolved in DMSO at a concentration up to 4 M and stored at −70° C. until use.

In a second step, the Cy3-His was joined to a 4-amino-3-butyric acid linker. Briefly, the linker was dissolved in 0.1 M sodium borate buffer pH 8.5 and Cy3-His dissolved in a small amount of neutral water was added in aliquots until equimolar concentrations were reached. After incubation for 2 hours at room temperature, the labeled product was purified by reverse phase HPLC on a Chemcobond 5C18 ODS column (4.6×150 mm). Elution was carried out with a linear gradient of 6% to 50% acetonitrile in 50 mM ammonium formate, pH 7.0, over a period of 30 min at a flow rate 1.0 mL/min, with monitoring of the eluate at 550 nm. The yield was 46%. HRMS (MALDI) calculated for C₄₄H₅₅N₆O₁₁S₂ [M⁺] was 908.0906 and found was 908.0955. The Cy3-His-4-amino-3-butyric acid (N,N-dimethylamino) ethylester was synthesized by Lewatit lipase EL1-mediated esterification as described above. The final product was purified by reverse phase HPLC, as described above, except that the gradient was 6% to 80% acetonitrile. The yield was 0.1%. HRMS (MALDI) calculated for C₅₃H₆₄N₉O₁₃S₂ [M⁺] was 1099.2815 and found was 1099.2841.

### 4. Preparation of His-tagged 5-bromo-4-chloro-3-indolyl-beta-galactopyranoside (X-Gal)

A histidine molecule was firstly incorporated to the X-Gal by enzymatic esterification of histidine with X-Gal and immobilized Thermomyces lanuginosus lipase EL1 (11.2 mol % incorporation of histidine in 24 h, at a ratio 2 histidine:1 X-Gal). 3-Methyl-2-butanol was used as solvent with a water content of up to 3.0%, and the reaction was carried out at 45° C. High-performance preparative liquid chromatography using nucleosil C18 column using methanol:water as mobile phase was used to analyze and purify the products. Purified X-Gal-His was stored at −20° C. until use. This method was used to link His molecules to carboxylate containing molecules.

### 5. Incorporation of X-Gal-His to Cy3-His-4-amino-3-butyric acid (N,N-dimethylamino) ethylester

The activated Cy3 is reacted with X-Gal-His halide in the presence of a quaternary ammonium salt such as benzyltriethylammonium chloride using 50% aqueous sodium hydroxide as an acid-removing agent (Bull. Chem. Soc. Jpn., 54, 1879 (1981)). By using this method, the Cy3 molecule is attached via the linker moiety to the halogenated moiety in the X-Gal molecule.

### 6. Incorporation of His-tagged X-Gal-Cy3 derivatives to the poly(A)-nitrilotriacetic-Co(II) complex

The final step in the array development is the incubation of the histidine functionalized Cy3-X-Gal with poly(A)-nitrilotriacetic-Co(II) complexes in 50 mM phosphate buffer, 50 mM NaCl, pH 7.5 for 1 hour at 25° C. When required, DMSO was added to increase substrate solubility. The resulting complexes were separated from unbound enzyme molecules by HPLC as described above. To ensure that each SM-Cy3 binds through both of its His residues, ⁵⁹NMR was performed, and only derivatives incorporating single SM-Cy3 molecules were purified, and stored in 384 microtiter plates at −70° C. until used. These molecules were used directly for the construction of the array.

### Example 5

### Improved Procedure for the Synthesis of Probe Compounds

### 1. Preparation of nitrilotriacetic-Co(II) complexes

The procedure was adapted from J. M. Abad et al., J Am Chem Soc 127, 5689 (2005), where the full synthesis is described. The amino-nitrilotriacetic-Co(II) complex (A) was formed by reaction of NR,NR-bis(carboxymethyl)-L-lysine hydrate (ANTA, Fluka) (5 g; 19 mmol) with an excess of cobalt(II) chloride (Sigma) (24.7 g; 190 mmol) in 20 mM HEPES in aqueous solution (10 ml total volume) overnight at 25° C. Excess cobalt was precipitated by increasing the pH to 10 by adding 4 N NaOH, and the precipitate was removed by filtration through a 0.2 μm membrane (PTFE, Amicon) (2.3 g, 53% yield; white solid crystalline powder) (A).

### Reaction Scheme:

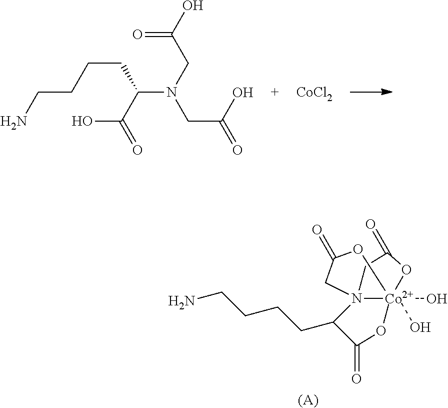

### 2. Incorporation of poly(A) tails to nitrilotriacetic-Co(II) complexes

The procedure was adapted from J. M. Abad et al., J Am Chem Soc 127, 5689 (2005). Briefly, activation of the phosphate groups of the poly(A) tails (Sigma Genosys) (2.091 mg; 258 nmol) and subsequent amidation with the Co(II) complex was performed by overnight incubation at 25° C. with 3 mM N-hydroxysuccinimide (NHS, Fluka) and 3 mM 1-ethyl-3-[3-(dimethylamino)propyl]-carbodiimide (EDC, Sigma) in 20 mM HEPES buffer (pH 7.5). Concentration of the pure fractions (freeze drier) afforded the poly(A)-ANTA-Co(II) complexes (B) as a white solid crystalline powder (1.82 mg; ˜86%).

### Reaction Scheme:

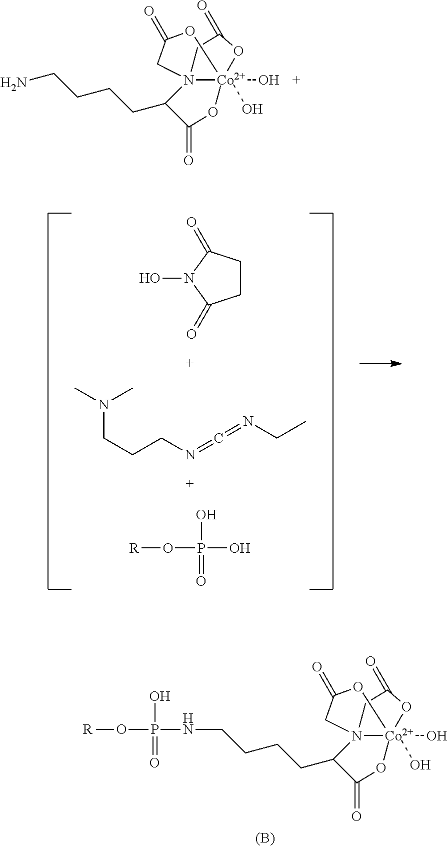

### 3. Modification of cyanine dye with histidine

A histidine molecule was firstly incorporated to the Cy3 dye by enzymatic transamidolysis of Cy3 NHS (a succinimidyl ester, GE Healthcare) with histidine and immobilized Lewatit lipase EL1 (D. Reyes-Duarte et al., Angew Chem Int Ed Engl 44, 7553 (2005)) from a cow rumen metagenome. All reactions were performed in the dark. Solvents were dried over 3 Å molecular sieves for 24 h prior to use. Briefly, Cy3 NHS (250 mg, 0.344 mmol) was dissolved in 5 mL of dimethylsulfoxide (DMSO) and 2-methyl-2-butanol was slowly added to a final volume of 25 mL. The biocatalyst (Lewatit lipase EL1, 2.5 g) and 3 Å molecular sieves (2.5 g) were then added and the suspension maintained 30 min at 40° C. with magnetic stirring. Then, histidine (Sigma) (213 mg; 1.38 mmol) was added. When the conversion of Cy3 NHS to the corresponding amide reached the maximum value (determined by HPLC: 37.39 mol % incorporation of histidine in 24 h, at a ratio 4 histidine:1 Cy3 NHS ester), the mixture was cooled, filtered and washed with 2-methyl-2-butanol. The crude product was purified by semipreparative reverse-phase HPLC (Prontosil-AQ, 5 μm, 120 A, 250·8 mm column), and compounds were eluted with acetonitrile (20% for 5 min, followed by linear gradients to 45% in 5 min, to 50% in 7 min and to 100% in 2 min) in triethylammonium hydrogen carbonate buffer (0.01 M, pH 8.6) at a flow of 3 mL/min. Fractions containing the product (C) (retention time 10.43 min, UV detection at 280 nm and 545 nm) were combined and dried by lyophilisation. The product (C) (101 mg; ˜37%) was further dissolved in dimethylsulfoxide (DMSO) at a concentration up to 4 M and stored at −70° C. until use. The water solubility of the compound was found to be >0.38 M at room temperature as determined by absorbance. The compound (C) had an extinction coefficient (ε) of 130810 l/mol·cm an its λₘₐₓ 545 nm as determined in 10 mM Tris-HCl at pH 7.4 according to the method of C. R. Cantor and I. Tinoco, J. Mol. Biol. 13, 65 (1965). Mass spectrometry was used to confirm the structure. HRMS: calculated for C₃₇O₉S₂N₅H₄₄ [M⁺H⁺] was 766.2580. found 766.2597. As shown, the result was within of the calculated molecular mass.

### Reaction Scheme:

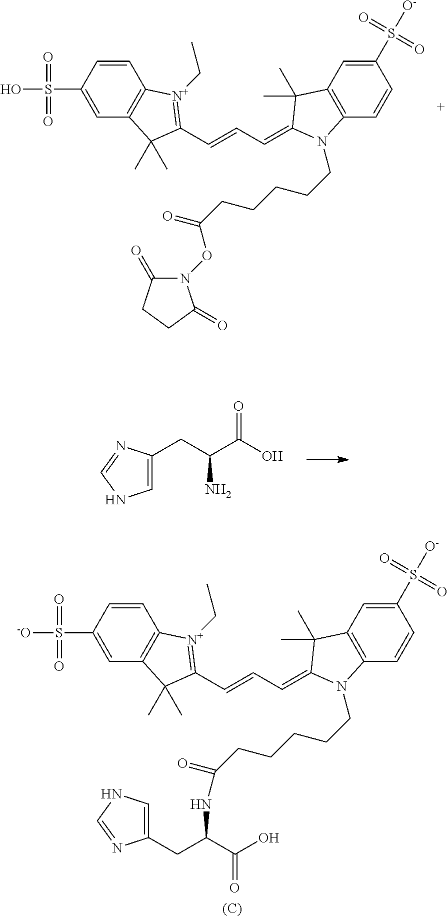

### 4. Modification of his tagged cyanine with 4-amino-3-butanoate

The 4-amino-3-butanoate was incorporated to (C) through the sulfonate. Two methods were used. First, sulfonyl chloride his tagged cyanine was firstly prepared from (C) according to J. Sokolowska-Gajda and H. S. Freeman, Dyes and Pigm 14, 35 (1990). This compound was used for a sulfonamide reaction (R—NH—SO₂-Cy3) with 4-amino-3-butanoate as described by Z. Wai et al., The proceeding of the 3rd International Conference on Functional Molecules 167 (2005). Briefly, to a solution of 4-amino-3-butanoate (60 mg; 0.58 mmol) in dry acetonitrile (10 ml), K₂CO₃ (1.0 g) was added and then a suspension of sulfonyl chloride Cy3-His (10 mg; 0.013 mmol) in dry DMSO (15 ml) was added dropwise. Then, the reaction mixture was stirred at 40° C. for 4 hrs and maintained for further 2 hrs. Then, the reaction mixture was cooled to room temperature, poured into water and extracted with ethyl acetate EtOAc. The solid obtained was dried under vacuum to give the product (D) as a light red powder. After recrystallized from acetonitrile, solid sulfonamide was obtained with the yield 32.0% (3.6 mg). The product was further purified by HPLC as described below. Additionally, the sulphonamide reaction was also performed as described by C. Tsopelas et al., J Nucl Med 43, 1377 (2002) with small modifications. Briefly, 4-amino-3-butanoate (60 mg; 0.58 mmol) was added to a 10 ml 0.1 M NaHCO₃ (pH 8.0) solution containing sulfonate Cy3-His (10 mg; 0.013 mmol) and further incubated at 40° C. for 600 min with swirling (100 rpm) in the dark. Under these conditions, 27 mol % incorporation of the dye was achieved. The product was recovered by semipreparative reverse phase HPLC analysis performed on a VPODS C-18 column (150×4.6 mm) at a flow rate of 1.0 mL/min for analysis, and PRC-ODS C-18 column (250×20 mm) at a flow rate of 10.0 mL/min for preparative scale. Detection was performed at 552 nm. HPLC solvents consist of water containing 0.1% TFA (solvent A) and acetonitrile containing 0.1% TFA (solvent B). Concentration of the pure fractions in vacuo afforded purified product (D) (2.7 mg; ˜24%). In both cases, purified product was further dissolved in dimethylsulfoxide (DMSO) at a concentration up to 4 M and stored at −70° C. until use. The mass spectrometry data was in agreement with the formation of one sulfonamide bond. HRMS: calculated for C₄₁O₁₀S₂N₆H₅₂ [M⁺H⁺] was 852.3186. found 852.3152.

### Reaction Scheme:

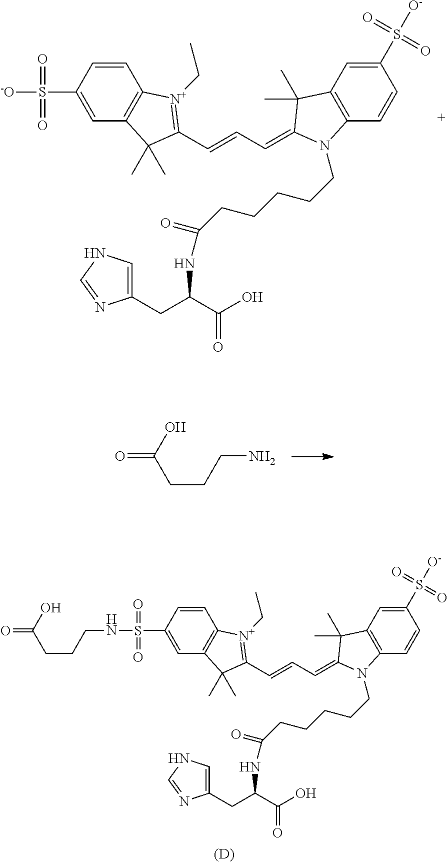

### 5. Modification of intermediate (D) with N,N-dimethylethanolamine

Compound (D) was subjected to direct esterification with N,N-dimethylethanolamine using immobilized Lewatit lipase EL1 (D. Reyes-Duarte et al., Angew Chem Int Ed Engl 44, 7553 (2005)) from a cow rumen metagenome. Briefly, compound (D) (25 mg, 0.029 mmol) was dissolved in 5 mL of dimethylsulfoxide (DMSO) and 2-methyl-2-butanol was slowly added to a final volume of 25 mL. The biocatalyst (Lewatit lipase EL1, 2.5 g) and 3 Å molecular sieves (2.5 g) were then added and the suspension maintained 30 min at 40° C. with magnetic stirring. Then, N,N-dimethylethanolamine (25 mg; 0.29 mmol) was added. When the conversion of the corresponding ester reached the maximum value (determined by HPLC: 53 mol % in 48 h), the mixture was cooled, filtered and washed with 2-methyl-2-butanol. The product was recovered by evaporating the tert-amyl alcohol. The crude product was redisolved in DMSO and further purified by semipreparative reverse-phase HPLC (Prontosil-AQ, 5 μm, 120 A, 250·8 mm column equipped with a Prontosil-AQ, 5 μm, 120 A, 33·8 mm pre-column, and compounds were eluted with acetonitrile (20% for 5 min, followed by linear gradients to 45% in 5 min, to 50% in 7 min and to 100% in 2 min) in triethylammonium hydrogen carbonate buffer (0.01 M, pH 8.6) at a flow of 3 mL/min. Fractions containing the product (E) (retention time 14.79 min, UV detection at 280 nm and 542 nm) were combined and dried by lyophilisation. The product (E) (13.5 mg; ˜50%) was further dissolved in dimethylsulfoxide (DMSO) at a concentration up to 4 M and stored at −70° C. until use. The water solubility of the compound was found to be approx. 0.24 M at room temperature as determined by absorbance. The compound (E) had an extinction coefficient (6) of 112728 l/mol·cm an its λₘₐₓ 545 nm as determined in 10 mM Tris-HCl at pH 7.4 according to the method of C. R. Cantor and I. Tinoco (loc. cit.). Mass spectrometry was used to confirm the structure. H HRMS: calculated for C₄₅O₁₀S₂N₇H₆₁ [M⁺H⁺] was 923.3921. found 923.3950. As shown, the result was within of the calculated molecular mass.

### Reaction Scheme:

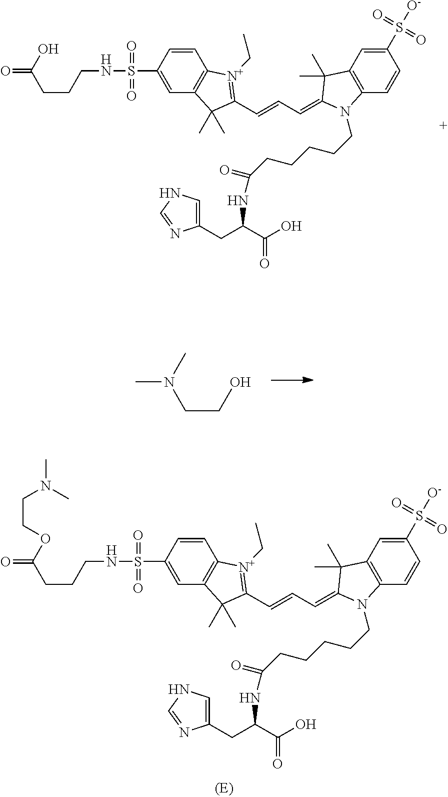

### 6. Metabolite Library

The primary synthetic challenge involved finding a reaction path from a functionalized core with the highest yield (>26-90% overall). The purified metabolites were characterized by HPLC and HRMS and were found to be 90% pure; yields were >20%. First, the comprehensive collection of metabolites was obtained. Table 1 shows a complete description of synthetic and purification methods and commercial suppliers. In case the metabolite was purified from cell extract of an organism, the chromatographic description of the method used for separation and the amount of metabolite per gram of extract are specifically shown. When a synthetic method was used, the exact and experimental masses (HRMS (MALDI)) are shown, together with the precise information of mass and molar quantities of reagents used, reaction conditions, work-up procedure and isolated yield in mass as well as percentage. As can be seen in Table S1 enzymatic and chemical methods were used. Metabolites were reconstituted and diluted in PBS buffer, DMSO or a mixture of both, accordingly to their solubility properties, and stored in 384-well microtiter plates at −70° C. until used.

### Reaction/Purification/Source Scheme:

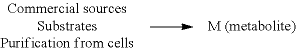

### 7. Histidine and dye labelled metabolite library synthesis

The metabolite array is constituted by thousands of molecules that are modified to link them to a fluorofore (i.e. Cy3, or possibly any other with similar characteristics) and a histidine molecule. Overall, there are a number of strategies to perform those addition reactions based on the linking and nature positions which are described below. Briefly, the methods include specific halogenation reactions by the action of a promiscuous halogenase, esterification or transesterification or amidation or transamidation reactions by the action of a promiscuous lipase and carbon-carbon bond formation in the presence of the proton sponge 1,8-bis-(dimethylamino)-napthalene. FIG. 8 shows a schematic representation of each of the different general methods used to create the histidine and dye labelled metabolite library synthesis. In this figure a representative metabolite is shown.

Even though approx. 2500 molecules were modified to anchor them to a dye, few dozen of general procedures can be considered to perform the synthesis, named “synthetic method 1 to 30” described below. These general methods are based on the nature of position of the molecules to which the histidine moiety and the dye moiety are attached. Table 8 shows the linking position to which both components are attached to the metabolites. Below the general synthetic methods are described, each of them describing the individual and successive steps. Further, general HPLC purification methods to separate the final dye labelled metabolites are described.

A number of abbreviations are used below: DMSO (dimethylsulfoxide); 2M2B (2-methyl-2-butanol); 1,8-BDN (1,8-bis-(dimethylamino)-naphthalene); CH₃CN (acetonitrile), α-KG (α-ketoglutarate), HPLC (high pressure liquid chromatography), EL1-Lewatit (immobilized Lewatit lipase EL1 from a cow rumen metagenome), 1-metabolite (Iodine containing metabolite; E (Cy3 intermediate containing histidine and linkers).

The source of enzymes used for synthetic purposes are as follow. EL1, Protein-engineered lipase isolated from cow rumen metagenome (Angewandte Chemie International Edition (2005) 44: 7553-7557); Dehalogenase: multifunctional α/β-hydrolase mined from a metagenome library of a microbial community in seawater contaminated with petroleum hydrocarbons, with a novel hydrolytic phenotype, namely the cleavage of both ‘common’ p-nitrophenyl (pNP) esters and haloalkanoates, and weak activity towards haloalkanes (Table 5; SEQ ID Nos. 57 to 59; paper in preparation); Halogenase: this enzyme corresponds to the same dehalogenase described above but it is able to perform halogenation reactions in organic media. The promiscuity of the enzyme can be also seen in the capacity to perform two different reactions. In the presence of the cofactor α-ketoglutarate the enzyme is able to activate non-activated alkyl groups and in the presence of NADH is able to halogenate activate molecules containing alkenyl groups.

Below we described the general methods which consist in three steps: first, the incorporation of iodide to the metabolite; second, the incorporation oh histidine to the halogenated metabolite; third, the incorporation of the dye to the previous intermediate. While the first and second methods may differs among the different methods, the third remains equal:

Step 3. Formation of Cy3-Labeled Metabolite.

The corresponding labeled quaternary ammonium metabolite were obtained in the presence of 1,8-BDN as described by R. A. Kaufman et al. (loc. cit.) and F. Mazzetti, R. M. Lemmon, J Org Chem 22, 228 (1957) with small modifications. Briefly, the general method for the synthesis of quaternary amines is as follows. Reaction mixture (2 ml) contains histidine tagged I-metabolite (0.078 mmol), 0.78 mmol of (E) and 1,8-BDN at a final concentration of 100 mM in DMSO. The temperature was controlled at 32° C. After incubation (range from 11.52 to 424 min) the product was recovered by evaporating the CH₃CN. The labeled product was purified by semi-preparative reverse-phase HPLC. The purified metabolite was found to be 98% pure.

Therefore, a full description of steps 1 and 2 is only given below. Reference is made to FIG. 8, which shows exemplary reaction schemes for each of the following 26 synthetic methods, and FIG. 9, which shows the exemplary metabolite used in the respective synthetic methods, wherein the position, to which histidine is linked, is marked by a black arrow, and that, to which the dye is linked, is marked by a grey arrow.

Synthetic Method 1:

this method is designed to perform direct halogenation to two sp3 carbon atoms (e.g. a carbon atom of a saturated hydrocarbon bond, e.g. a terminal methyl carbon atom or a methylene carbon atom, e.g. within a chain or ring) and further incorporation of His (black arrow in FIG. 9) and Cy3 (grey arrow in FIG. 9) component to those sp3 carbon atoms—i.e. both components to two different sp3 carbon atoms.

Example of metabolite subjected to this synthetic protocol is the metabolite nr. 1 shown in Table 1 and FIGS. 8 and 9, wherein Hys and Cy3 are linked to two different sp3 carbon atoms of an alkyl group.

1. Formation of 1-metabolite.

The iodide halogenation of metabolite was performed via α-KG. The halogenation reaction contained the metabolite (2.1 mM), KI (5.0 mM), and halogenase (32 μM) in phosphate buffer (20 mM, pH 7.8) plus 2 mM α-KG and up to 5% DMSO, in a final volume of 0.5 ml. After 10 h of incubation at 37° C., the reaction mixture was transferred to a 1.5 ml filtration unit (3000 NMWL membrane cut-off) to remove the enzyme, and the product was purified by semi preparative HPLC. I-metabolite was reconstituted and diluted in DMSO and stored at −70° C. until used at a concentration of 10 μg/ml. HRMS data clearly show that the enzyme incorporated two “I” per molecule.

2. Incorporation of Histidine.

I-metabolite was further functionalized via incorporation of a histidine tag. The incorporation of histidine was performed in the presence of the non-nucleophilic base and proton sponge 1,8-BDN as described by R. A. Kaufman et al. (loc. cit.) and F. Mazzetti and R. M. Lemmon (loc. cit.) with small modifications. Briefly, to a solution of 1-metabolite (concentration range from 0.1 to 2.3 mmol), histidine (concentration range from 0.44 to 1 mmol), and 1,8-BDN at a final concentration of 10 mM were added, in CH₃CN (10 ml final volume). The temperature was controlled at 32° C. After incubation (range from 11.52 to 424 min) the product was recovered by evaporating the CH₃CN. The corresponding product was purified by semi-preparative HPLC. The purified metabolite was found to be 98% pure.

Synthetic Method 2:

this method is designed to perform direct halogenation to two sp2 carbon atoms (e.g. a carbon atom of an aromatic carbon bond or a carbon atom of an unsaturated hydrocarbon bond, e.g. terminal, or within a chain or ring) and further incorporation of His (black arrow in FIG. 9) and Cy3 (grey arrow in FIG. 9) component to those groups—i.e. both components to two different sp2 carbon atoms.

Example of metabolite subjected to this synthetic protocol is the metabolite nr. 7 shown in Table 1 and FIGS. 8 and 9, wherein His and Cy3 are linked to two different sp2 carbon atoms of an aryl group.

1. Formation of 1-Metabolite.

The iodide halogenation of metabolite was performed via I⁻ as follows. The general halogenation procedure via cofactor is as follows. The reaction mixtures were incubated at 37° C. with metabolite (0.080 mmol), KI (75 mM), 2 mM NADH, and halogenase (4 mg/ml, 100 μL) in phosphate buffer (20 mM, pH=7.8), containing up to 20% DMSO (to increase metabolite solubility), in a final volume of 0.5 ml. After 24 hour of incubation, reaction product(s) were separated by semi-preparative HPLC. I-metabolite was reconstituted and diluted in DMSO and stored at −70° C. until used at a concentration of 10 μg/ml. HRMS data clearly show that the enzyme incorporated two “I” per molecule.

2. Incorporation of Histidine.

I-metabolite was further functionalized via incorporation of a histidine tag. The incorporation of histidine was performed in the presence of the non-nucleophilic base and proton sponge 1,8-BDN as described by R. A. Kaufman et al. (loc. cit.) and F. Mazzetti and R. M. Lemmon (loc. cit.) with small modifications. Briefly, to a solution of 1-metabolite (concentration range from 0.1 to 2.3 mmol), histidine (concentration range from 0.44 to 1 mmol), and 1,8-BDN at a final concentration of 10 mM were added, in CH₃CN (10 ml final volume). The temperature was controlled at 32° C. After incubation (range from 11.52 to 424 min) the product was recovered by evaporating the CH₃CN. The corresponding product was purified by semi-preparative HPLC. The purified metabolite was found to be 98% pure.

Synthetic Method 3:

this method is designed to perform direct halogenation to both a sp3 carbon atom (e.g. a carbon atom of a saturated hydrocarbon bond, e.g. a terminal methyl carbon atom or a methylene carbon atom, e.g. within a chain or ring) and a sp2 carbon atom (e.g. a carbon atom of an aromatic carbon bond or a carbon atom of an unsaturated hydrocarbon bond, e.g. terminal, or within a chain or ring) in the same metablite and further incorporation of His (black arrow in FIG. 9) and Cy3 (grey arrow in FIG. 9) component to those groups—i.e. one component to a sp3 carbon atom and one component to a sp2 carbon atom. Example of metabolite subjected to this synthetic protocol is the metabolite nr. 8 shown in Table 1 and FIGS. 8 and 9, wherein one of His and Cy3 is linked to a sp3 carbon atom of an methyl group, while the other is linked to an aryl carbon atom.

1. Formation of 1-Metabolite.

The iodide halogenation of metabolite was performed via I⁻ as follows. The general halogenation procedure via cofactor is as follows. The reaction mixtures were incubated at 37° C. with metabolite (0.080 mmol), KI (75 mM), 2 mM NADH, and halogenase (4 mg/ml, 100 μl) in phosphate buffer (20 mM, pH=7.8), containing up to 20% DMSO (to increase metabolite solubility), in a final volume of 0.5 ml. After 24 hour of incubation, reaction product(s) were separated by semi-preparative HPLC. I-metabolite was reconstituted and diluted in DMSO and stored at −70° C. until used at a concentration of 10 μg/ml. HRMS data clearly show that the enzyme incorporated one “I” per molecule. Further, a second iodide was incorporated via α-KG. The halogenation reaction contained the metabolite (2.1 mM), KI (5.0 mM), and halogenase (32 μM) in phosphate buffer (20 mM, pH 7.8) plus 2 mM α-KG and up to 5% DMSO, in a final volume of 0.5 ml. After 10 h of incubation at 37° C., the reaction mixture was transferred to a 1.5 ml filtration unit (3000 NMWL membrane cut-off) to remove the enzyme, and the product was purified by semi preparative HPLC. I-metabolite was reconstituted and diluted in DMSO and stored at −70° C. until used at a concentration of 10 μg/ml. HRMS data clearly show that the enzyme incorporated one “I” per molecule in this step, and two “I” per molecule at the end of the two-steps halogenation process.

2. Incorporation of Histidine.

I-metabolite was further functionalized via incorporation of a histidine tag. The incorporation of histidine was performed in the presence of the non-nucleophilic base and proton sponge 1,8-BDN as described by R. A. Kaufman et. al. (loc. cit.) and F. Mazzetti and R. M. Lemmon (loc. cit.) with small modifications. Briefly, to a solution of 1-metabolite (concentration range from 0.1 to 2.3 mmol), histidine (concentration range from 0.44 to 1 mmol), and 1,8-BDN at a final concentration of 10 mM were added were added, in CH₃CN (10 ml final volume). The temperature was controlled at 32° C. After incubation (range from 11.52 to 424 min) the product was recovered by evaporating the CH₃CN. The corresponding product was purified by semi-preparative HPLC. The purified metabolite was found to be 98% pure.

Synthetic Method 4:

this method is designed to perform direct halogenation to a sp3 carbon atom (e.g. a carbon atom of a saturated hydrocarbon bond, e.g. a terminal methyl carbon atom or a methylene carbon atom, e.g. within a chain or ring) to link it to Cy3 (grey arrow in FIG. 9) component and incorporation of His (black arrow in FIG. 9) through esterification with an OH group of the metabolite—i.e. one component to a sp3 carbon atom and one component to an OH group.

Example of metabolite subjected to this synthetic protocol is the metabolite nr. 36 shown in Table 1 and FIGS. 8 and 9.

1. Formation of 1-Metabolite.

The iodide halogenation of metabolite was performed via α-KG. The halogenation reaction contained the metabolite (2.1 mM), KI (5.0 mM), and halogenase (32 μM) in phosphate buffer (20 mM, pH 7.8) plus 2 mM α-KG and up to 5% DMSO, in a final volume of 0.5 ml. After 10 h of incubation at 37° C., the reaction mixture was transferred to a 1.5 ml filtration unit (3000 NMWL membrane cut-off) to remove the enzyme, and the product was purified by semi-preparative HPLC. I-metabolite was reconstituted and diluted in DMSO and stored at −70° C. until used at a concentration of 10 μg/ml. HRMS data clearly show that the enzyme incorporated one “I” per molecule.

2. Incorporation of Histidine.

I-metabolite was further functionalized via incorporation of a histidine tag. A histidine molecule was incorporated by enzymatic esterification of the previous intermediate and EL1-Lewatit. Solvents were dried over 3 Å molecular sieves for 24 h prior to use. Briefly, metabolite (0.344 mmol) was dissolved in 5 mL of DMSO and 2M2B was slowly added to a final volume of 25 mL. The biocatalyst (Lewatit lipase EL1, 2.5 g) and 3 Å molecular sieves (2.5 g) were then added and the suspension maintained 30 min at 40° C. with magnetic stirring. Then, histidine (1.38 mmol) was added. When the conversion to the corresponding ester reached the maximum value, the mixture was cooled, filtered and washed with 2M2B. The product was recovered by evaporating the tert-amyl alcohol and the crude product was purified by semi-preparative HPLC. Fractions containing the product were combined and dried by lyophilisation. I-metabolite was reconstituted and diluted in DMSO and stored at −70° C. until used at a concentration of 10 μg/ml.

Synthetic Method 5:

this method is designed to perform direct esterification through an —OH group and further halogenation to a sp3 carbon atom (e.g. a carbon atom of a saturated hydrocarbon bond, e.g. a terminal methyl carbon atom or a methylene carbon atom, e.g. within a chain or ring) to link it to Cy3 (grey arrow in FIG. 9) component and His (black arrow in FIG. 9)—i.e. both components to two different OH groups.

Example of metabolite subjected to this synthetic protocol is the metabolite nr. 132 shown in Table 1 and FIGS. 8 and 9.

1. Formation of 1-Metabolite.

The metabolite was subjected to transesterification with vinyl acetate using EL1-Lewatit. Briefly, metabolite (0.029 mmol) was dissolved in 5 mL of DMSO and 2M2B was slowly added to a final volume of 25 mL. The biocatalyst (Lewatit lipase EL1, 2.5 g) and 3 Å molecular sieves (2.5 g) were then added and the suspension maintained 30 min at 40° C. with magnetic stirring. Then, vinyl acetate (0.29 mmol) was added. When the conversion of the corresponding ester reached the maximum value, the mixture was cooled, filtered and washed with 2M2B. The product was recovered by evaporating the tert-amyl alcohol. The crude product was further purified by semi-preparative HPLC. The product so obtained was further subjected to iodide halogenation via α-KG. The halogenation reaction contained the metabolite (2.1 mM), KI (5.0 mM), and halogenase (32 μl) in phosphate buffer (20 mM, pH 7.8) plus 2 mM α-KG and up to 5% DMSO, in a final volume of 0.5 ml. After 10 h of incubation at 37° C., the reaction mixture was transferred to a 1.5 ml filtration unit (3000 NMWL membrane cut-off) to remove the enzyme, and the product was purified by semi-preparative HPLC. I-metabolite was reconstituted and diluted in DMSO and stored at −70° C. until used at a concentration of 10 μg/ml.

2. Incorporation of Histidine.

I-metabolite was further functionalized via incorporation of a histidine tag. A histidine molecule was incorporated by enzymatic esterification of the previous intermediate and EL1-Lewatit. Solvents were dried over 3 Å molecular sieves for 24 h prior to use. Briefly, metabolite (0.344 mmol) was dissolved in 5 mL of DMSO and 2M2B was slowly added to a final volume of 25 mL. The biocatalyst (Lewatit lipase EL1, 2.5 g) and 3 Å molecular sieves (2.5 g) were then added and the suspension maintained 30 min at 40° C. with magnetic stirring. Then, histidine (1.38 mmol) was added. When the conversion to the corresponding ester reached the maximum value, the mixture was cooled, filtered and washed with 2M2B. The product was recovered by evaporating the tert-amyl alcohol and the crude product was purified by semi-preparative HPLC. Fractions containing the product were combined and dried by lyophilisation. I-metabolite was reconstituted and diluted in DMSO and stored at −70° C. until used at a concentration of 10 μg/ml.

Synthetic Method 6:

this method is designed to perform direct halogenation to a sp3 carbon atom (e.g. a carbon atom of a saturated hydrocarbon bond, e.g. a terminal methyl carbon atom or a methylene carbon atom, e.g. within a chain or ring) to link it to Cy3 (grey arrow in FIG. 9) component and incorporation of His (black arrow in FIG. 9) through an NH₂ group of the metabolite—i.e. one component to a sp3 carbon atom and one component to an NH₂ group. Example of metabolite subjected to this synthetic protocol is the metabolite nr. 154 shown in Table 1 and FIGS. 8 and 9.

1. Formation of 1-Metabolite.

The iodide halogenation of metabolite was performed via α-KG. The halogenation reaction contained the metabolite (2.1 mM), KI (5.0 mM), and halogenase (32 μM) in phosphate buffer (20 mM, pH 7.8) plus 2 mM α-KG and up to 5% DMSO, in a final volume of 0.5 ml. After 10 h of incubation at 37° C., the reaction mixture was transferred to a 1.5 ml filtration unit (3000 NMWL membrane cut-off) to remove the enzyme, and the product was purified by semi-preparative HPLC. I-metabolite was reconstituted and diluted in DMSO and stored at −70° C. until used at a concentration of 10 μg/ml. HRMS data clearly show that the enzyme incorporated one “I” per molecule.

2. Incorporation of Histidine.

I-metabolite was further functionalized via incorporation of a histidine tag. A histidine molecule was incorporated by enzymatic amidation of the previous intermediate and EL1-Lewatit. Solvents were dried over 3 Å molecular sieves for 24 h prior to use. Briefly, metabolite (0.344 mmol) was dissolved in 5 mL of DMSO and 2M2B was slowly added to a final volume of 25 mL. The biocatalyst (Lewatit lipase EL1, 2.5 g) and 3 Å molecular sieves (2.5 g) were then added and the suspension maintained 30 min at 40° C. with magnetic stirring. Then, histidine (1.38 mmol) was added. When the conversion to the corresponding amide reached the maximum value, the mixture was cooled, filtered and washed with 2M2B. The crude product was further purified by semi-preparative HPLC. I-metabolite was reconstituted and diluted in DMSO and stored at −70° C. until used at a concentration of 10 μg/ml.

Synthetic Method 7:

this method is designed to perform direct esterification through an —OH group and further halogenation to a sp3 carbon atom to link it to Cy3 (grey arrow in FIG. 9) component and incorporation of His (black arrow in FIG. 9) through an NH₂ group of the metabolite—i.e. one component to an NH₂ group and one component to an OH group.

Example of metabolite subjected to this synthetic protocol is the metabolite nr. 196 shown in Table 1 and FIGS. 8 and 9.

1. Formation of 1-Metabolite.

The metabolite was subjected to transesterification with vinyl acetate using EL1-Lewatit. Briefly, metabolite (0.029 mmol) was dissolved in 5 mL of DMSO and 2M2B was slowly added to a final volume of 25 mL. The biocatalyst (Lewatit lipase EL1, 2.5 g) and 3 Å molecular sieves (2.5 g) were then added and the suspension maintained 30 min at 40° C. with magnetic stirring. Then, vinyl acetate (0.29 mmol) was added. When the conversion of the corresponding ester reached the maximum value, the mixture was cooled, filtered and washed with 2M2B. The product was recovered by evaporating the tert-amyl alcohol. The crude product was further purified by semi-preparative HPLC. The product so obtained was further subjected to iodide halogenation via α-KG. The halogenation reaction contained the metabolite (2.1 mM), KI (5.0 mM), and halogenase (32 μM) in phosphate buffer (20 mM, pH 7.8) plus 2 mM α-KG and up to 5% DMSO, in a final volume of 0.5 ml. After 10 h of incubation at 37° C., the reaction mixture was transferred to a 1.5 ml filtration unit (3000 NMWL membrane cut-off) to remove the enzyme, and the product was purified by semi-preparative HPLC. I-metabolite was reconstituted and diluted in DMSO and stored at −70° C. until used at a concentration of 10 μg/ml.

2. Incorporation of Histidine.

I-metabolite was further functionalized via incorporation of a histidine tag. A histidine molecule was incorporated by enzymatic amidation of the previous intermediate and EL1-Lewatit. Solvents were dried over 3 Å molecular sieves for 24 h prior to use. Briefly, metabolite (0.344 mmol) was dissolved in 5 mL of DMSO and 2M2B was slowly added to a final volume of 25 mL. The biocatalyst (Lewatit lipase EL1, 2.5 g) and 3 Å molecular sieves (2.5 g) were then added and the suspension maintained 30 min at 40° C. with magnetic stirring. Then, histidine (1.38 mmol) was added. When the conversion to the corresponding amide reached the maximum value, the mixture was cooled, filtered and washed with 2M2B. The crude product was further purified by semi-preparative HPLC. I-metabolite was reconstituted and diluted in DMSO and stored at −70° C. until used at a concentration of 10 μg/ml.

Synthetic Method 8:

this method is designed to perform partial dehalogenation in (fully) halogenated aliphatic compounds, followed by halogenation (iodization) to two different sp3 carbon atoms (e.g. a carbon atom of a saturated hydrocarbon bond, e.g. a terminal methyl carbon atom or a methylene carbon atom, e.g. within a chain or ring) and further incorporation of His (black arrow in FIG. 9) and Cy3 (grey arrow in FIG. 9) component to those sp3 carbon atoms—i.e. both components to two different sp3 carbon atoms after a three-step dehalogenation, esterification and halogenation.

Example of metabolite subjected to this synthetic protocol is the metabolite nr. 1692 shown in Table 1 and FIGS. 8 and 9.

1. Formation of 1-Metabolite.

The iodide halogenation of metabolite was performed via α-KG prior selective dehalogenation. First, the metabolite (100 μl of a 100 mM solution in MeOH) was added to 900 μl in phosphate buffer (20 mM, pH 7.8) and dehalogenase (32 μM). Reaction was allowed to proceed at 40° C. for 5 hours. Then the reaction mixture was transferred to a 1.5 ml filtration unit (3000 NMWL membrane cut-off) to remove the enzyme, and the product (mixture of partial dehalogenated products) was concentrated by liophilization. The purified intermediate (95% pure) was used for a subsequent iodization reaction as follows. The metabolite was subjected to transesterification with vinyl acetate using EL1-Lewatit. Briefly, metabolite (0.029 mmol) was dissolved in 5 mL of DMSO and 2M2B was slowly added to a final volume of 25 mL. The biocatalyst (Lewatit lipase EL1, 2.5 g) and 3 Å molecular sieves (2.5 g) were then added and the suspension maintained 30 min at 40° C. with magnetic stirring. Then, vinyl acetate (0.29 mmol) was added. When the conversion of the corresponding ester reached the maximum value, the mixture was cooled, filtered and washed with 2M2B. The product was recovered by evaporating the tert-amyl alcohol. The crude product was further purified by semi-preparative HPLC. The product so obtained was further subjected to iodide halogenation via α-KG. The halogenation reaction contained the metabolite (2.1 mM), KI (5.0 mM), and halogenase (32 μM) in phosphate buffer (20 mM, pH 7.8) plus 2 mM α-KG and up to 5% DMSO, in a final volume of 0.5 ml. After 10 h of incubation at 37° C., the reaction mixture was transferred to a 1.5 ml filtration unit (3000 NMWL membrane cut-off) to remove the enzyme, and the product was purified by semi-preparative HPLC. I-metabolite was reconstituted and diluted in DMSO and stored at −70° C. until used at a concentration of 10 μg/ml. HRMS data clearly show that the enzyme incorporated two “I” per molecule.

2. Incorporation of Histidine.

I-metabolite was further functionalized via incorporation of a histidine tag. The incorporation of histidine was performed in the presence of the non-nucleophilic base and proton sponge 1,8-BDN as described by R. A. Kaufman et al. (loc. cit.) and F. Mazzetti and R. M. Lemmon (loc. cit.) with small modifications. Briefly, to a solution of 1-metabolite (concentration range from 0.1 to 2.3 mmol), histidine (concentration range from 0.44 to 1 mmol), and 1,8-BDN at a final concentration of 10 mM were added were added, in CH₃CN (10 ml final volume). The temperature was controlled at 32° C. After incubation (range from 11.52 to 424 min) the product was recovered by evaporating the CH₃CN. The corresponding product was purified by semi-preparative HPLC. The purified metabolite was found to be 98% pure.

Synthetic Method 9:

this method is designed to perform direct halogenation to a sp2 carbon atom (e.g. a carbon atom of an aromatic carbon bond or a carbon atom of an unsaturated hydrocarbon bond, e.g. terminal, or within a chain or ring) and further incorporation of Cy3 (grey arrow in FIG. 9) component to this group and incorporation of His (black arrow in FIG. 9) through an NH₂ group of the metabolite.

Example of metabolite subjected to this synthetic protocol is the metabolite nr. 306 shown in Table 1 and FIGS. 8 and 9.

1. Formation of 1-Metabolite.

The iodide halogenation of metabolite was performed via I⁻ as follows. The general halogenation procedure via cofactor is as follows. The reaction mixtures were incubated at 37° C. with metabolite (0.080 mmol), KI (75 mM), 2 mM NADH, and halogenase (4 mg/ml, 100 μL) in phosphate buffer (20 mM, pH=7.8), containing up to 20% DMSO (to increase metabolite solubility), in a final volume of 0.5 ml. After 24 hour of incubation, reaction product(s) were separated by semi-preparative HPLC. I-metabolite was reconstituted and diluted in DMSO and stored at −70° C. until used at a concentration of 10 μg/ml. HRMS data clearly show that the enzyme incorporated one “I” per molecule after purification.

2. Incorporation of Histidine.

I-metabolite was further functionalized via incorporation of a histidine tag. A histidine molecule was incorporated by enzymatic amidation of the previous intermediate and EL1-Lewatit. Solvents were dried over 3 Å molecular sieves for 24 h prior to use. Briefly, metabolite (0.344 mmol) was dissolved in 5 mL of DMSO and 2M2B was slowly added to a final volume of 25 mL. The biocatalyst (Lewatit lipase EL1, 2.5 g) and 3 Å molecular sieves (2.5 g) were then added and the suspension maintained 30 min at 40° C. with magnetic stirring. Then, histidine (1.38 mmol) was added. When the conversion to the corresponding amide reached the maximum value, the mixture was cooled, filtered and washed with 2M2B. The product was recovered by evaporating the tert-amyl alcohol and the crude product was purified by semi-preparative HPLC. Fractions containing the product were combined and dried by lyophilisation. I-metabolite was reconstituted and diluted in DMSO and stored at −70° C. until used at a concentration of 10 μg/ml.

Synthetic Method 10:

this method is designed to perform direct esterification through a —COOH group and further halogenation to a sp3 carbon atom to link it to Cy3 (grey arrow in FIG. 9) component and incorporation of His (black arrow in FIG. 9) through halogenation to a sp3 carbon atom (e.g. a carbon atom of a saturated hydrocarbon bond, e.g. a terminal methyl carbon atom or a methylene carbon atom, e.g. within a chain or ring).

Example of metabolite subjected to this synthetic protocol is the metabolite nr. 327 shown in Table 1 and FIGS. 8 and 9.

1. Formation of 1-Metabolite.

The metabolite was subjected to direct esterification with ethanol using EL1-Lewatit. Briefly, metabolite (0.029 mmol) was dissolved in 5 mL of DMSO and 2M2B was slowly added to a final volume of 25 mL. The biocatalyst (Lewatit lipase EL1, 2.5 g) and 3 Å molecular sieves (2.5 g) were then added and the suspension maintained 30 min at 40° C. with magnetic stirring. Then, ethanol (0.29 mmol) was added. When the conversion of the corresponding ester reached the maximum value, the mixture was cooled, filtered and washed with 2M2B. The product was recovered by evaporating the tert-amyl alcohol. The crude product was further purified by semi-preparative HPLC. The product so obtained was further subjected to iodide halogenation via α-KG. The halogenation reaction contained the metabolite (2.1 mM), KI (5.0 mM), and halogenase (32 μM) in phosphate buffer (20 mM, pH 7.8) plus 2 mM α-KG and up to 5% DMSO, in a final volume of 0.5 ml. After 10 h of incubation at 37° C., the reaction mixture was transferred to a 1.5 ml filtration unit (3000 NMWL membrane cut-off) to remove the enzyme, and the product was purified by semi-preparative HPLC. I-metabolite was reconstituted and diluted in DMSO and stored at −70° C. until used at a concentration of 10 μg/ml. Following the two-step reaction process, HRMS data clearly show that the enzyme incorporated two “I” per molecule after purification.

2. Incorporation of Histidine.

I-metabolite was further functionalized via incorporation of a histidine tag. The incorporation of histidine was performed in the presence of the non-nucleophilic base and proton sponge 1,8-BDN as described by R. A. Kaufman et al. (loc. cit.) and F. Mazzetti and R. M. Lemmon (loc. cit.) with small modifications. Briefly, to a solution of 1-metabolite (concentration range from 0.1 to 2.3 mmol), histidine (concentration range from 0.44 to 1 mmol), and 1,8-BDN at a final concentration of 10 mM were added, in CH₃CN (10 ml final volume). The temperature was controlled at 32° C. After incubation (range from 11.52 to 424 min) the product was recovered by evaporating the CH₃CN. The corresponding product was purified by semi-preparative HPLC. The purified metabolite was found to be 98% pure.

Synthetic Method 11:

this method is designed to perform direct esterification through a —COOH group and further halogenation to a sp3 carbon atom to link it to Cy3 (grey arrow in FIG. 9) component and incorporation of His (black arrow in FIG. 9) through an —OH group of the metabolite. Example of metabolite subjected to this synthetic protocol is the metabolite nr. shown in Table 1 and FIGS. 8 and 9.

1. Formation of 1-Metabolite.

The metabolite was subjected to direct esterification with ethanol using EL1-Lewatit. Briefly, metabolite (0.029 mmol) was dissolved in 5 mL of DMSO and 2M2B was slowly added to a final volume of 25 mL. The biocatalyst (Lewatit lipase EL1, 2.5 g) and 3 Å molecular sieves (2.5 g) were then added and the suspension maintained 30 min at 40° C. with magnetic stirring. Then, ethanol (0.29 mmol) was added. When the conversion of the corresponding ester reached the maximum value, the mixture was cooled, filtered and washed with 2M2B. The product was recovered by evaporating the tert-amyl alcohol. The crude product was further purified by semi-preparative HPLC. The product so obtained was further subjected to iodide halogenation via α-KG. The halogenation reaction contained the metabolite (2.1 mM), KI (5.0 mM), and halogenase (32 μM) in phosphate buffer (20 mM, pH 7.8) plus 2 mM α-KG and up to 5% DMSO, in a final volume of 0.5 ml. After 10 h of incubation at 37° C., the reaction mixture was transferred to a 1.5 ml filtration unit (3000 NMWL membrane cut-off) to remove the enzyme, and the product was purified by semi-preparative HPLC. I-metabolite was reconstituted and diluted in DMSO and stored at −70° C. until used at a concentration of 10 μg/ml. HRMS data clearly show that the enzyme incorporated one “I” per molecule

2. Incorporation of Histidine.

I-metabolite was further functionalized via incorporation of a histidine tag. A histidine molecule was incorporated by enzymatic esterification of the previous intermediate and EL1-Lewatit. Solvents were dried over 3 Å molecular sieves for 24 h prior to use. Briefly, metabolite (0.344 mmol) was dissolved in 5 mL of DMSO and 2M2B was slowly added to a final volume of 25 mL. The biocatalyst (Lewatit lipase EL1, 2.5 g) and 3 Å molecular sieves (2.5 g) were then added and the suspension maintained 30 min at 40° C. with magnetic stirring. Then, histidine (1.38 mmol) was added. When the conversion to the corresponding ester reached the maximum value, the mixture was cooled, filtered and washed with 2M2B. The crude product was further purified by semi-preparative HPLC. I-metabolite was reconstituted and diluted in DMSO and stored at −70° C. until used at a concentration of 10 μg/ml.

Synthetic Method 12:

this method is designed to perform direct esterification through two —COOH groups and further halogenation to a sp3 carbon atom to link them to Cy3 (grey arrow in FIG. 9) and His (black arrow in FIG. 9) components.

Example of metabolite subjected to this synthetic protocol is the metabolite nr. 397 shown in Table 1 and FIGS. 8 and 9.

1. Formation of 1-Metabolite.

The metabolite was subjected to direct esterification with ethanol using EL1-Lewatit. Briefly, metabolite (0.029 mmol) was dissolved in 5 mL of DMSO and 2M2B was slowly added to a final volume of 25 mL. The biocatalyst (Lewatit lipase EL1, 2.5 g) and 3 Å molecular sieves (2.5 g) were then added and the suspension maintained 30 min at 40° C. with magnetic stirring. Then, ethanol (0.29 mmol) was added. When the conversion of the corresponding ester reached the maximum value, the mixture was cooled, filtered and washed with 2M2B. The product was recovered by evaporating the tert-amyl alcohol. The crude product was further purified by semi-preparative HPLC. The product so obtained was further subjected to iodide halogenation via α-KG. The halogenation reaction contained the metabolite (2.1 mM), KI (5.0 mM), and halogenase (32 μM) in phosphate buffer (20 mM, pH 7.8) plus 2 mM α-KG and up to 5% DMSO, in a final volume of 0.5 ml. After 10 h of incubation at 37° C., the reaction mixture was transferred to a 1.5 ml filtration unit (3000 NMWL membrane cut-off) to remove the enzyme, and the product was purified by semi-preparative HPLC. I-metabolite was reconstituted and diluted in DMSO and stored at −70° C. until used at a concentration of 10 μg/ml. HRMS data clearly show that the enzyme incorporated two “I” per molecule.

2. Incorporation of Histidine.

I-metabolite was further functionalized via incorporation of a histidine tag. The incorporation of histidine was performed in the presence of the non-nucleophilic base and proton sponge 1,8-BDN as described by R. A. Kaufman et al. (loc. cit.) and F. Mazzetti and R. M. Lemmon (loc. cit.) with small modifications. Briefly, to a solution of 1-metabolite (concentration range from 0.1 to 2.3 mmol), histidine (concentration range from 0.44 to 1 mmol), and 1,8-BDN at a final concentration of 10 mM were added, in CH₃CN (10 ml final volume). The temperature was controlled at 32° C. After incubation (range from 11.52 to 424 min) the product was recovered by evaporating the CH₃CN. The corresponding product was purified by semi-preparative HPLC. The purified metabolite was found to be 98% pure.

Synthetic Method 13:

this method is designed to perform direct halogenation to a sp2 carbon atom (e.g. a carbon atom of an aromatic carbon bond or a carbon atom of an unsaturated hydrocarbon bond, e.g. terminal, or within a chain or ring) and further incorporation of Cy3 (grey arrow in FIG. 9) component and direct amidation through a —COOH group to incorporate the His (black arrow in FIG. 9) component.

Example of metabolite subjected to this synthetic protocol is the metabolite nr. 398 shown in Table 1 and FIGS. 8 and 9.

1. Formation of 1-Metabolite.

The iodide halogenation of metabolite was performed via I⁻ as follows. The general halogenation procedure via cofactor is as follows. The reaction mixtures were incubated at 37° C. with metabolite (0.080 mmol), KI (75 mM), 2 mM NADH, and halogenase (4 mg/ml, 100 in phosphate buffer (20 mM, pH=7.8), containing up to 20% DMSO (to increase metabolite solubility), in a final volume of 0.5 ml. After 24 hour of incubation, reaction product(s) were separated by semi-preparative HPLC. I-metabolite was reconstituted and diluted in DMSO and stored at −70° C. until used at a concentration of 10 μg/ml. HRMS data clearly show that the enzyme incorporated one “I” per molecule.

2. Incorporation of Histidine.

I-metabolite was further functionalized via incorporation of a histidine tag. A histidine molecule was incorporated by enzymatic amidation of the previous intermediate and EL1-Lewatit. Solvents were dried over 3 Å molecular sieves for 24 h prior to use. Briefly, metabolite (0.344 mmol) was dissolved in 5 mL of DMSO and 2M2B was slowly added to a final volume of 25 mL. The biocatalyst (Lewatit lipase EL1, 2.5 g) and 3 Å molecular sieves (2.5 g) were then added and the suspension maintained 30 min at 40° C. with magnetic stirring. Then, histidine (1.38 mmol) was added. When the conversion to the corresponding amide reached the maximum value, the mixture was cooled, filtered and washed with 2M2B. The product was recovered by evaporating the tert-amyl alcohol and the crude product was purified by semi-preparative HPLC. Fractions containing the product were combined and dried by lyophilisation. I-metabolite was reconstituted and diluted in DMSO and stored at −70° C. until used at a concentration of 10 μg/ml.

Synthetic Method 14:

this method is designed to perform direct esterification through a —COOH group and further halogenation to a sp3 carbon atom to link it to Cy3 (grey arrow in FIG. 9) and direct amidation through a NH₂ group to incorporate the His (black arrow in FIG. 9) component.

Example of metabolite subjected to this synthetic protocol is the metabolite nr. 860 shown in Table 1 and FIGS. 8 and 9.

1. Formation of 1-Metabolite.

The metabolite was subjected to direct esterification with ethanol using EL1-Lewatit. Briefly, metabolite (0.029 mmol) was dissolved in 5 mL of DMSO and 2M2B was slowly added to a final volume of 25 mL. The biocatalyst (Lewatit lipase EL1, 2.5 g) and 3 Å molecular sieves (2.5 g) were then added and the suspension maintained 30 min at 40° C. with magnetic stirring. Then, ethanol (0.29 mmol) was added. When the conversion of the corresponding ester reached the maximum value, the mixture was cooled, filtered and washed with 2M2B. The product was recovered by evaporating the tert-amyl alcohol. The crude product was further purified by semi-preparative HPLC. The product so obtained was further subjected to iodide halogenation via α-KG. The halogenation reaction contained the metabolite (2.1 mM), KI (5.0 mM), and halogenase (32 μM) in phosphate buffer (20 mM, pH 7.8) plus 2 mM α-KG and up to 5% DMSO, in a final volume of 0.5 ml. After 10 h of incubation at 37° C., the reaction mixture was transferred to a 1.5 ml filtration unit (3000 NMWL membrane cut-off) to remove the enzyme, and the product was purified by semi-preparative HPLC. I-metabolite was reconstituted and diluted in DMSO and stored at −70° C. until used at a concentration of 10 μg/ml. HRMS data clearly show that the enzyme incorporated one “I” per molecule.

2. Incorporation of Histidine.

I-metabolite was further functionalized via incorporation of a histidine tag. A histidine molecule was incorporated by enzymatic amidation of the previous intermediate and EL1-Lewatit. Solvents were dried over 3 Å molecular sieves for 24 h prior to use. Briefly, metabolite (0.344 mmol) was dissolved in 5 mL of DMSO and 2M2B was slowly added to a final volume of 25 mL. The biocatalyst (Lewatit lipase EL1, 2.5 g) and 3 Å molecular sieves (2.5 g) were then added and the suspension maintained 30 min at 40° C. with magnetic stirring. Then, histidine (1.38 mmol) was added. When the conversion to the corresponding amide reached the maximum value, the mixture was cooled, filtered and washed with 2M2B. The product was recovered by evaporating the tert-amyl alcohol and the crude product was purified by semi-preparative HPLC. Fractions containing the product were combined and dried by lyophilisation. I-metabolite was reconstituted and diluted in DMSO and stored at −70° C. until used at a concentration of 10 μg/ml.

Synthetic Method 15:

this method is designed to perform direct transesterification through a —OH group and further halogenation to a sp3 carbon atom to link it to Cy3 (grey arrow in FIG. 9) and direct amidation through a NH₂ group to incorporate the His (black arrow in FIG. 9) component.

Example of metabolite subjected to this synthetic protocol is the metabolite nr. 543 shown in Table 1 and FIGS. 8 and 9.

1. Formation of 1-Metabolite.

The metabolite was subjected to transesterification with vinyl acetate using EL1-Lewatit. Briefly, metabolite (0.029 mmol) was dissolved in 5 mL of DMSO and 2M2B was slowly added to a final volume of 25 mL. The biocatalyst (Lewatit lipase EL1, 2.5 g) and 3 Å molecular sieves (2.5 g) were then added and the suspension maintained 30 min at 40° C. with magnetic stirring. Then, vinyl acetate (0.29 mmol) was added. When the conversion of the corresponding ester reached the maximum value, the mixture was cooled, filtered and washed with 2M2B. The product was recovered by evaporating the tert-amyl alcohol. The crude product was further purified by semi-preparative HPLC. The product so obtained was further subjected to iodide halogenation via α-KG. The halogenation reaction contained the metabolite (2.1 mM), KI (5.0 mM), and halogenase (32 μM) in phosphate buffer (20 mM, pH 7.8) plus 2 mM α-KG and up to 5% DMSO, in a final volume of 0.5 ml. After 10 h of incubation at 37° C., the reaction mixture was transferred to a 1.5 ml filtration unit (3000 NMWL membrane cut-off) to remove the enzyme, and the product was purified by semi-preparative HPLC. I-metabolite was reconstituted and diluted in DMSO and stored at −70° C. until used at a concentration of 10 μg/ml. HRMS data clearly show that the enzyme incorporated one “I” per molecule.

2. Incorporation of Histidine.

I-metabolite was further functionalized via incorporation of a histidine tag. A histidine molecule was incorporated by enzymatic amidation of the previous intermediate and EL1-Lewatit. Solvents were dried over 3 Å molecular sieves for 24 h prior to use. Briefly, metabolite (0.344 mmol) was dissolved in 5 mL of DMSO and 2M2B was slowly added to a final volume of 25 mL. The biocatalyst (Lewatit lipase EL1, 2.5 g) and 3 Å molecular sieves (2.5 g) were then added and the suspension maintained 30 min at 40° C. with magnetic stirring. Then, histidine (1.38 mmol) was added. When the conversion to the corresponding ester reached the maximum value, the mixture was cooled, filtered and washed with 2M2B. The product was recovered by evaporating the tert-amyl alcohol and the crude product was purified by semi-preparative HPLC. Fractions containing the product were combined and dried by lyophilisation. I-metabolite was reconstituted and diluted in DMSO and stored at −70° C. until used at a concentration of 10 μg/ml.

Synthetic Method 16:

this method is designed to perform direct transesterification through a —OH group and further halogenation to a sp3 carbon atom to link it to Cy3 (grey arrow in FIG. 9) component and incorporation of His (black arrow in FIG. 9) through a —COOH group of the metabolite.

Example of metabolite subjected to this synthetic protocol is the metabolite nr. 1179 shown in Table 1 and FIGS. 8 and 9.

1. Formation of 1-Metabolite.

The metabolite was subjected to direct transesterification with vinyl acetate using EL1-Lewatit. Briefly, metabolite (0.029 mmol) was dissolved in 5 mL of DMSO and 2M2B was slowly added to a final volume of 25 mL. The biocatalyst (Lewatit lipase EL1, 2.5 g) and 3 Å molecular sieves (2.5 g) were then added and the suspension maintained 30 min at 40° C. with magnetic stirring. Then, vinyl acetate (0.29 mmol) was added. When the conversion of the corresponding ester reached the maximum value, the mixture was cooled, filtered and washed with 2M2B. The product was recovered by evaporating the tert-amyl alcohol. The crude product was further purified by semi-preparative HPLC. The product so obtained was further subjected to iodide halogenation via α-KG. The halogenation reaction contained the metabolite (2.1 mM), KI (5.0 mM), and halogenase (32 μM) in phosphate buffer (20 mM, pH 7.8) plus 2 mM α-KG and up to 5% DMSO, in a final volume of 0.5 ml. After 10 h of incubation at 37° C., the reaction mixture was transferred to a 1.5 ml filtration unit (3000 NMWL membrane cut-off) to remove the enzyme, and the product was purified by semi-preparative HPLC. I-metabolite was reconstituted and diluted in DMSO and stored at −70° C. until used at a concentration of 10 μg/ml. HRMS data clearly show that the enzyme incorporated one “I” per molecule.

2. Incorporation of Histidine.

I-metabolite was further functionalized via incorporation of a histidine tag. A histidine molecule was incorporated by enzymatic amidation of the previous intermediate and EL1-Lewatit. Solvents were dried over 3 Å molecular sieves for 24 h prior to use. Briefly, metabolite (0.344 mmol) was dissolved in 5 mL of DMSO and 2M2B was slowly added to a final volume of 25 mL. The biocatalyst (Lewatit lipase EL1, 2.5 g) and 3 Å molecular sieves (2.5 g) were then added and the suspension maintained 30 min at 40° C. with magnetic stirring. Then, histidine (1.38 mmol) was added. When the conversion to the corresponding amide reached the maximum value, the mixture was cooled, filtered and washed with 2M2B. The product was recovered by evaporating the tert-amyl alcohol and the crude product was purified by semi-preparative HPLC. Fractions containing the product were combined and dried by lyophilisation. I-metabolite was reconstituted and diluted in DMSO and stored at −70° C. until used at a concentration of 10 μg/ml.

Synthetic Method 17:

this method is designed to perform direct esterification through one —OH group and further halogenation to a sp3 carbon atom to link it to Cy3 (grey arrow in FIG. 9) and His (black arrow in FIG. 9) components.

Example of metabolite subjected to this synthetic protocol is the metabolite nr. 1212 shown in Table 1 and FIGS. 8 and 9.

1. Formation of 1-Metabolite.

The metabolite was subjected to direct transesterification with vinyl propionate using EL1-Lewatit. Briefly, metabolite (0.029 mmol) was dissolved in 5 mL of DMSO and 2M2B was slowly added to a final volume of 25 mL. The biocatalyst (Lewatit lipase EL1, 2.5 g) and 3 Å molecular sieves (2.5 g) were then added and the suspension maintained 30 min at 40° C. with magnetic stirring. Then, vinyl propionate (0.29 mmol) was added. When the conversion of the corresponding ester reached the maximum value, the mixture was cooled, filtered and washed with 2M2B. The product was recovered by evaporating the tert-amyl alcohol. The crude product was further purified by semi-preparative HPLC. The product so obtained was further subjected to iodide halogenation via α-KG. The halogenation reaction contained the metabolite (2.1 mM), KI (5.0 mM), and halogenase (32 μM) in phosphate buffer (20 mM, pH 7.8) plus 2 mM α-KG and up to 5% DMSO, in a final volume of 0.5 ml. After 10 h of incubation at 37° C., the reaction mixture was transferred to a 1.5 ml filtration unit (3000 NMWL membrane cut-off) to remove the enzyme, and the product was purified by semi-preparative HPLC. I-metabolite was reconstituted and diluted in DMSO and stored at −70° C. until used at a concentration of 10 μg/ml. HRMS data clearly show that the enzyme incorporated two “I” per molecule.

2. Incorporation of Histidine.

I-metabolite was further functionalized via incorporation of a histidine tag. The incorporation of histidine was performed in the presence of the non-nucleophilic base and proton sponge 1,8-BDN as described by R. A. Kaufman et al. (loc. cit.) and F. Mazzetti and R. M. Lemmon (loc. cit.) with small modifications. Briefly, to a solution of 1-metabolite (concentration range from 0.1 to 2.3 mmol), histidine (concentration range from 0.44 to 1 mmol), and 1,8-BDN at a final concentration of 10 mM were added, in CH₃CN (10 ml final volume). The temperature was controlled at 32° C. After incubation (range from 11.52 to 424 min) the product was recovered by evaporating the CH₃CN. The corresponding product was purified by semi-preparative HPLC. The purified metabolite was found to be 98% pure.

Synthetic Method 18:

this method is designed to perform direct halogenation to one sp3 carbon atom (e.g. a carbon atom of a saturated hydrocarbon bond, e.g. a terminal methyl carbon atom or a methylene carbon atom, e.g. within a chain or ring) and further incorporation of His (black arrow in FIG. 9) and Cy3 (grey arrow in FIG. 9) components to this moiety

Example of metabolite subjected to this synthetic protocol is the metabolite nr. 417 shown in Table 1 and FIGS. 8 and 9.

1-1. Formation of 1-Metabolite I.

The iodide halogenation of metabolite was performed via α-KG. The halogenation reaction contained the metabolite (2.1 mM), KI (5.0 mM), and halogenase (32 μM) in phosphate buffer (20 mM, pH 7.8) plus 2 mM α-KG and up to 5% DMSO, in a final volume of 0.5 ml. After 10 h of incubation at 37° C., the reaction mixture was transferred to a 1.5 ml filtration unit (3000 NMWL membrane cut-off) to remove the enzyme, and the product was purified by semi-preparative HPLC. I-metabolite was reconstituted and diluted in DMSO and stored at −70° C. until used at a concentration of 10 μg/ml. HRMS data clearly show that the enzyme incorporated one “I” per molecule.

1-2. Incorporation of Ethyl Amine I-Metabolite I.

The corresponding metabolite was used to incorporate an ethyl group throught the reaction with ethyl amine in the presence of 1,8-BDN as described by R. A. Kaufman et al. (loc. cit.) and F. Mazzetti and R. M. Lemmon (loc. cit.) with small modifications. Briefly, reaction mixture (2 ml) contains I-metabolite I (0.078 mmol), 0.78 mmol of ethyl amine and 1,8-BDN at a final concentration of 100 mM in DMSO (10 ml final volume). The temperature was controlled at 32° C. After incubation (range from 11.52 to 424 min) the product was recovered by evaporating the CH₃CN. The labeled product was purified by semi-preparative HPLC. The purified metabolite was found to be 98% pure.

1-3. Formation of 1-Metabolite II.

The iodide halogenation of metabolite was performed via α-KG. The halogenation reaction contained the metabolite (2.1 mM), KI (5.0 mM), and halogenase (32 μM) in phosphate buffer (20 mM, pH 7.8) plus 2 mM α-KG and up to 5% DMSO, in a final volume of 0.5 ml. After 10 h of incubation at 37° C., the reaction mixture was transferred to a 1.5 ml filtration unit (3000 NMWL membrane cut-off) to remove the enzyme, and the product was purified by semi-preparative HPLC. I-metabolite was reconstituted and diluted in DMSO and stored at −70° C. until used at a concentration of 10 μg/ml. HRMS data clearly show that the enzyme incorporated two “I” per molecule.

2. Incorporation of Histidine.

I-metabolite was further functionalized via incorporation of a histidine tag. The incorporation of histidine was performed in the presence of the non-nucleophilic base and proton sponge 1,8-BDN as described by R. A. Kaufman and et al. (loc. cit.) and F. Mazzetti and R. M. Lemmon (loc. cit.) with small modifications. Briefly, to a solution of 1-metabolite (concentration range from 0.1 to 2.3 mmol), histidine (concentration range from 0.44 to 1 mmol), and 1,8-BDN at a final concentration of 10 mM were added, in CH₃CN (10 ml final volume). The temperature was controlled at 32° C. After incubation (range from 11.52 to 424 min) the product was recovered by evaporating the CH₃CN. The corresponding product was purified by semi-preparative HPLC. The purified metabolite was found to be 98% pure.

Synthetic Method 19:

this method is designed to perform direct esterification through a —COOH group and further halogenation to a sp3 carbon atom to link it to Cy3 (grey arrow in FIG. 9) and direct halogenation to a sp2 carbon atom (e.g. a carbon atom of an aromatic carbon bond or a carbon atom of an unsaturated hydrocarbon bond, e.g. terminal, or within a chain or ring) and further incorporation of His (grey arrow in FIG. 9) component Example of metabolite subjected to this synthetic protocol is the metabolite nr. 1332 shown in Table 1 and FIGS. 8 and 9.

1. Formation of 1-Metabolite.

The metabolite was subjected to direct esterification with ethanol using EL1-Lewatit. Briefly, metabolite (0.029 mmol) was dissolved in 5 mL of DMSO and 2M2B was slowly added to a final volume of 25 mL. The biocatalyst (Lewatit lipase EL1, 2.5 g) and 3 Å molecular sieves (2.5 g) were then added and the suspension maintained 30 min at 40° C. with magnetic stirring. Then, ethanol (0.29 mmol) was added. When the conversion of the corresponding ester reached the maximum value, the mixture was cooled, filtered and washed with 2M2B. The product was recovered by evaporating the tert-amyl alcohol. The crude product was further purified by semi-preparative HPLC. The product so obtained was further subjected to iodide halogenation via α-KG. The halogenation reaction contained the metabolite (2.1 mM), KI (5.0 mM), and halogenase (32 μM) in phosphate buffer (20 mM, pH 7.8) plus 2 mM α-KG and up to 5% DMSO, in a final volume of 0.5 ml. After 10 h of incubation at 37° C., the reaction mixture was transferred to a 1.5 ml filtration unit (3000 NMWL membrane cut-off) to remove the enzyme, and the product was purified by semi-preparative HPLC. I-metabolite was reconstituted and diluted in DMSO and stored at −70° C. until used at a concentration of 10 μg/ml. Using this intermediate a second iodization reaction was performed via I⁻. The general halogenation procedure via cofactor is as follows. The reaction mixtures were incubated at 37° C. with metabolite (0.080 mmol), KI (75 mM), 2 mM NADH, and halogenase (4 mg/ml, 100 μL) in phosphate buffer (20 mM, pH=7.8), containing up to 20% DMSO (to increase metabolite solubility), in a final volume of 0.5 ml. After 24 hour of incubation, reaction product(s) were separated by semi-preparative HPLC. I-metabolite was reconstituted and diluted in DMSO and stored at −70° C. until used at a concentration of 10 μg/ml. HRMS data clearly show that the enzyme incorporated two “I” per molecule after the two-step process.

2. Incorporation of Histidine.

I-metabolite was further functionalized via incorporation of a histidine tag. The incorporation of histidine was performed in the presence of the non-nucleophilic base and proton sponge 1,8-BDN as described by R. A. Kaufman et al. (loc. cit.) and F. Mazzetti and R. M. Lemmon (loc. cit.) with small modifications. Briefly, to a solution of 1-metabolite (concentration range from 0.1 to 2.3 mmol), histidine (concentration range from 0.44 to 1 mmol), and 1,8-BDN at a final concentration of 10 mM were added, in CH₃CN (10 ml final volume). The temperature was controlled at 32° C. After incubation (range from 11.52 to 424 min) the product was recovered by evaporating the CH₃CN. The corresponding product was purified by semi-preparative HPLC. The purified metabolite was found to be 98% pure.

Synthetic Method 20:

this method is designed to perform direct halogenation to a sp3 carbon atom (e.g. a carbon atom of a saturated hydrocarbon bond, e.g. a terminal methyl carbon atom or a methylene carbon atom, e.g. within a chain or ring) and further incorporation of Cy3 (grey arrow in FIG. 9) component to this alkyl group and direct amidation through a —COOH group to link it to His (grey arrow in FIG. 9) component.

Example of metabolite subjected to this synthetic protocol is the metabolite nr. 1790 shown in Table 1 and FIGS. 8 and 9.

1. Formation of 1-Metabolite.

The iodide halogenation of metabolite was performed via α-KG. The halogenation reaction contained the metabolite (2.1 mM), KI (5.0 mM), and halogenase (32 μM) in phosphate buffer (20 mM, pH 7.8) plus 2 mM α-KG and up to 5% DMSO, in a final volume of 0.5 ml. After 10 h of incubation at 37° C., the reaction mixture was transferred to a 1.5 ml filtration unit (3000 NMWL membrane cut-off) to remove the enzyme, and the product was purified by semi-preparative HPLC. I-metabolite was reconstituted and diluted in DMSO and stored at −70° C. until used at a concentration of 10 μg/ml. HRMS data clearly show that the enzyme incorporated one “I” per molecule.

2. Incorporation of Histidine.

I-metabolite was further functionalized via incorporation of a histidine tag. A histidine molecule was incorporated by enzymatic amidation of the previous intermediate and EL1-Lewatit. Solvents were dried over 3 Å molecular sieves for 24 h prior to use. Briefly, metabolite (0.344 mmol) was dissolved in 5 mL of DMSO and 2M2B was slowly added to a final volume of 25 mL. The biocatalyst (Lewatit lipase EL1, 2.5 g) and 3 Å molecular sieves (2.5 g) were then added and the suspension maintained 30 min at 40° C. with magnetic stirring. Then, histidine (1.38 mmol) was added. When the conversion to the corresponding amide reached the maximum value, the mixture was cooled, filtered and washed with 2M2B. The product was recovered by evaporating the tert-amyl alcohol and the crude product was purified by semi-preparative HPLC. Fractions containing the product were combined and dried by lyophilisation. I-metabolite was reconstituted and diluted in DMSO and stored at −70° C. until used at a concentration of 10 μg/ml.

Synthetic Method 21:

this method is designed to perform direct esterification through a —COOH group and further halogenation to a sp3 carbon atom to link it to Cy3 (grey arrow in FIG. 9) and His (grey arrow in FIG. 9) components.

Example of metabolite subjected to this synthetic protocol is the metabolite nr. 1409 shown in Table 1 and FIGS. 8 and 9.

1. Formation of 1-Metabolite.

The metabolite was subjected to direct esterification with ethanol using EL1-Lewatit. Briefly, metabolite (0.029 mmol) was dissolved in 5 mL of DMSO and 2M2B was slowly added to a final volume of 25 mL. The biocatalyst (Lewatit lipase EL1, 2.5 g) and 3 Å molecular sieves (2.5 g) were then added and the suspension maintained 30 min at 40° C. with magnetic stirring. Then, ethanol (0.29 mmol) was added. When the conversion of the corresponding ester reached the maximum value, the mixture was cooled, filtered and washed with 2M2B. The product was recovered by evaporating the tert-amyl alcohol. The crude product was further purified by semi-preparative HPLC. The product so obtained was further subjected to iodide halogenation via α-KG. The halogenation reaction contained the metabolite (2.1 mM), KI (5.0 mM), and halogenase (32 μM) in phosphate buffer (20 mM, pH 7.8) plus 2 mM α-KG and up to 5% DMSO, in a final volume of 0.5 ml. After 10 h of incubation at 37° C., the reaction mixture was transferred to a 1.5 ml filtration unit (3000 NMWL membrane cut-off) to remove the enzyme, and the product was purified by semi-preparative HPLC. I-metabolite was reconstituted and diluted in DMSO and stored at −70° C. until used at a concentration of 10 μg/ml. HRMS data clearly show that the enzyme incorporated two “I” per molecule.

2. Incorporation of Histidine.

I-metabolite was further functionalized via incorporation of a histidine tag. The incorporation of histidine was performed in the presence of the non-nucleophilic base and proton sponge 1,8-BDN as described by R. A. Kaufman et al. (loc. cit.) and F. Mazzetti and R. M. Lemmon (loc. cit.) with small modifications. Briefly, to a solution of 1-metabolite (concentration range from 0.1 to 2.3 mmol), histidine (concentration range from 0.44 to 1 mmol), and 1,8-BDN at a final concentration of 10 mM were added, in CH₃CN (10 ml final volume). The temperature was controlled at 32° C. After incubation (range from 11.52 to 424 min) the product was recovered by evaporating the CH₃CN. The corresponding product was purified by semi-preparative HPLC. The purified metabolite was found to be 98% pure.

Synthetic Method 22:

this method is designed to perform direct halogenation to one sp2 carbon atom (e.g. a carbon atom of an aromatic carbon bond or a carbon atom of an unsaturated hydrocarbon bond, e.g. terminal, or within a chain or ring) and further incorporation of His (black arrow in FIG. 9) and Cy3 (grey arrow in FIG. 9) components to this moiety.

Example of metabolite subjected to this synthetic protocol is the metabolite nr. 1614 shown in Table 1 and FIGS. 8 and 9.

1-1. Formation of 1-Metabolite I.

The iodide halogenation of metabolite was performed via I⁻ as follows. The general halogenation procedure via cofactor is as follows. The reaction mixtures were incubated at 37° C. with metabolite (0.080 mmol), KI (75 mM), 2 mM NADH, and halogenase (4 mg/ml, 100 μL) in phosphate buffer (20 mM, pH=7.8), containing up to 20% DMSO (to increase metabolite solubility), in a final volume of 0.5 ml. After 24 hour of incubation, reaction product(s) were separated by semi-preparative HPLC. I-metabolite was reconstituted and diluted in DMSO and stored at −70° C. until used at a concentration of 10 μg/ml. HRMS data clearly show that the enzyme incorporated one “I” per molecule.

1-2. Incorporation of Ethyl Amine.

The corresponding metabolite was used to incorporate an ethyl group throught the reaction with ethyl amine in the presence of 1,8-BDN as described by R. A. Kaufman et al. (loc. cit.) and F. Mazzetti and R. M. Lemmon (loc. cit.) with small modifications. Briefly, reaction mixture (2 ml) contains I-metabolite (0.078 mmol), 0.78 mmol of ethyl amine and 1,8-BDN at a final concentration of 100 mM in DMSO (10 ml final volume). The temperature was controlled at 32° C. After incubation (range from 11.52 to 424 min) the product was recovered by evaporating the CH₃CN. The labeled product was purified by semi-preparative HPLC. The purified metabolite was found to be 98% pure.

1-3. Formation of 1-Metabolite II.

The iodide halogenation of metabolite was performed via α-KG. The halogenation reaction contained the metabolite (2.1 mM), KI (5.0 mM), and halogenase (32 μM) in phosphate buffer (20 mM, pH 7.8) plus 2 mM α-KG and up to 5% DMSO, in a final volume of 0.5 ml. After 10 h of incubation at 37° C., the reaction mixture was transferred to a 1.5 ml filtration unit (3000 NMWL membrane cut-off) to remove the enzyme, and the product was purified by semi-preparative HPLC. I-metabolite was reconstituted and diluted in DMSO and stored at −70° C. until used at a concentration of 10 μg/ml. HRMS data clearly show that the enzyme incorporated two “I” per molecule.

2. Incorporation of Histidine.

I-metabolite was further functionalized via incorporation of a histidine tag. The incorporation of histidine was performed in the presence of the non-nucleophilic base and proton sponge 1,8-BDN as described by R. A. Kaufman et al. (loc. cit.) and F. Mazzetti and R. M. Lemmon (loc. cit.) with small modifications. Briefly, to a solution of 1-metabolite (concentration range from 0.1 to 2.3 mmol), histidine (concentration range from 0.44 to 1 mmol), and 1,8-BDN at a final concentration of 10 mM were added, in CH₃CN (10 ml final volume). The temperature was controlled at 32° C. After incubation (range from 11.52 to 424 min) the product was recovered by evaporating the CH₃CN. The corresponding product was purified by semi-preparative HPLC. The purified metabolite was found to be 98% pure.

Synthetic Method 23:

this method is designed to perform direct halogenation to a sp3 carbon atom (e.g. a carbon atom of a saturated hydrocarbon bond, e.g. a terminal methyl carbon atom or a methylene carbon atom, e.g. within a chain or ring) and further incorporation of Cy3 (grey arrow in FIG. 9) component and direct amidation through a —COOH group to link it to His (grey arrow in FIG. 9) component.

Example of metabolite subjected to this synthetic protocol is the metabolite nr. 394 shown in Table 1 and FIGS. 8 and 9.

1. Formation of 1-Metabolite.

The iodide halogenation of metabolite was performed via α-KG. The halogenation reaction contained the metabolite (2.1 mM), KI (5.0 mM), and halogenase (32 μM) in phosphate buffer (20 mM, pH 7.8) plus 2 mM α-KG and up to 5% DMSO, in a final volume of 0.5 ml. After 10 h of incubation at 37° C., the reaction mixture was transferred to a 1.5 ml filtration unit (3000 NMWL membrane cut-off) to remove the enzyme, and the product was purified by semi-preparative HPLC. I-metabolite was reconstituted and diluted in DMSO and stored at −70° C. until used at a concentration of 10 μg/ml. HRMS data clearly show that the enzyme incorporated one “I” per molecule.

2. Incorporation of Histidine.

I-metabolite was further functionalized via incorporation of a histidine tag. A histidine molecule was incorporated by enzymatic amidation of the previous intermediate and EL1-Lewatit. Solvents were dried over 3 Å molecular sieves for 24 h prior to use. Briefly, metabolite (0.344 mmol) was dissolved in 5 mL of DMSO and 2M2B was slowly added to a final volume of 25 mL. The biocatalyst (Lewatit lipase EL1, 2.5 g) and 3 Å molecular sieves (2.5 g) were then added and the suspension maintained 30 min at 40° C. with magnetic stirring. Then, histidine (1.38 mmol) was added. When the conversion to the corresponding amide reached the maximum value, the mixture was cooled, filtered and washed with 2M2B. The product was recovered by evaporating the tert-amyl alcohol and the crude product was purified by semi-preparative HPLC. Fractions containing the product were combined and dried by lyophilisation. I-metabolite was reconstituted and diluted in DMSO and stored at −70° C. until used at a concentration of 10 μg/ml.

Synthetic Method 24:

this method is designed to perform partial dehalogenation in an halogenated compound, followed by halogenation (iodization) to an alkyl group and further incorporation of Cy3 (grey arrow) component to this alkyl group and direct esterification through one —OH group to link it to His (black arrow) component.

Example of metabolite subjected to this synthetic protocol is the metabolite nr. 1997 shown in Table 1 and FIG. 9.

1. Formation of 1-Metabolite.

The iodide halogenation of metabolite was performed via α-KG prior selective dehalogenation. First, the metabolite (100 μl of a 10 mM solution in MeOH final concentration of 1 mM) was added to 900 μl in phosphate buffer (20 mM, pH 7.8) and dehalogenase (32 μM). Reaction was allowed to proceed at 40° C. for 5 hours. Then the reaction mixture was transferred to a 1.5 ml filtration unit (3000 NMWL membrane cut-off) to remove the enzyme, and the product (mixture of partial dehalogenated products) was concentrated by liophilization. The purified intermediate (95% pure) was used for a subsequent iodization reaction as follows. The metabolite was subjected to transesterification with vinyl acetate using EL1-Lewatit. Briefly, metabolite (0.029 mmol) was dissolved in 5 mL of DMSO and 2M2B was slowly added to a final volume of 25 mL. The biocatalyst (Lewatit lipase EL1, 2.5 g) and 3 Å molecular sieves (2.5 g) were then added and the suspension maintained 30 min at 40° C. with magnetic stirring. Then, vinyl acetate (0.29 mmol) was added. When the conversion of the corresponding ester reached the maximum value, the mixture was cooled, filtered and washed with 2M2B. The product was recovered by evaporating the tert-amyl alcohol. The crude product was further purified by semi-preparative HPLC. The product so obtained was further subjected to iodide halogenation via α-KG. The halogenation reaction contained the metabolite (2.1 mM), KI (5.0 mM), and halogenase (32 μl) in phosphate buffer (20 mM, pH 7.8) plus 2 mM α-KG and up to 5% DMSO, in a final volume of 0.5 ml. After 10 h of incubation at 37° C., the reaction mixture was transferred to a 1.5 ml filtration unit (3000 NMWL membrane cut-off) to remove the enzyme, and the product was purified by semi-preparative HPLC. I-metabolite was reconstituted and diluted in DMSO and stored at −70° C. until used at a concentration of 10 μg/ml. HRMS data clearly show that the enzyme incorporated two “I” per molecule.

2. Incorporation of Histidine.

I-metabolite was further functionalized via incorporation of a histidine tag. The incorporation of histidine was performed in the presence of the non-nucleophilic base and proton sponge 1,8-BDN as described by R. A. Kaufman et al. (loc. cit.) and F. Mazzetti and R. M. Lemmon (loc. cit.) with small modifications. Briefly, to a solution of 1-metabolite (concentration range from 0.1 to 2.3 mmol), histidine (concentration range from 0.44 to 1 mmol), and 1,8-BDN at a final concentration of 10 mM were added were added, in CH₃CN (10 ml final volume). The temperature was controlled at 32° C. After incubation (range from 11.52 to 424 min) the product was recovered by evaporating the CH₃CN. The corresponding product was purified by semi-preparative HPLC. The purified metabolite was found to be 98% pure.

Synthetic Method 25:

this method is designed to perform direct transamidation through one —NH₂ group and further halogenation to a sp3 carbon atom to link it to Cy3 (grey arrow in FIG. 9) and His (black arrow in FIG. 9) components.

Example of metabolite subjected to this synthetic protocol is the metabolite nr. 1481 shown in Table 1 and FIGS. 8 and 9.

1. Formation of 1-Metabolite.

The metabolite was subjected to direct transamidation with vinyl propionate using EL1-Lewatit. Briefly, metabolite (0.029 mmol) was dissolved in 5 mL of DMSO and 2M2B was slowly added to a final volume of 25 mL. The biocatalyst (Lewatit lipase EL1, 2.5 g) and 3 Å molecular sieves (2.5 g) were then added and the suspension maintained 30 min at 40° C. with magnetic stirring. Then, acetate (0.29 mmol) was added. When the conversion of the corresponding amide reached the maximum value, the mixture was cooled, filtered and washed with 2M2B. The product was recovered by evaporating the tert-amyl alcohol. The crude product was further purified by semi-preparative HPLC. The product so obtained was further subjected to iodide halogenation via α-KG. The halogenation reaction contained the metabolite (2.1 mM), KI (5.0 mM), and halogenase (32 μM) in phosphate buffer (20 mM, pH 7.8) plus 2 mM α-KG and up to 5% DMSO, in a final volume of 0.5 ml. After 10 h of incubation at 37° C., the reaction mixture was transferred to a 1.5 ml filtration unit (3000 NMWL membrane cut-off) to remove the enzyme, and the product was purified by semi-preparative HPLC. I-metabolite was reconstituted and diluted in DMSO and stored at −70° C. until used at a concentration of 10 μg/ml. HRMS data clearly show that the enzyme incorporated two “I” per molecule at the end of the two-step process.

2. Incorporation of Histidine.

I-metabolite was further functionalized via incorporation of a histidine tag. The incorporation of histidine was performed in the presence of the non-nucleophilic base and proton sponge 1,8-BDN as described by R. A. Kaufman et al. (loc. cit.) and F. Mazzetti and R. M. Lemmon (loc. cit.) with small modifications. Briefly, to a solution of 1-metabolite (concentration range from 0.1 to 2.3 mmol), histidine (concentration range from 0.44 to 1 mmol), and 1,8-BDN at a final concentration of 10 mM were added, in CH₃CN (10 ml final volume). The temperature was controlled at 32° C. After incubation (range from 11.52 to 424 min) the product was recovered by evaporating the CH₃CN. The corresponding product was purified by semi-preparative HPLC. The purified metabolite was found to be 98% pure.

Synthetic Method 26:

this method is designed to perform partial dehalogenation in an halogenated unsaturated compound, followed by halogenation (iodization) to a sp3 carbon atom and further incorporation of Cy3 (grey arrow in FIG. 9) component to this sp3 carbon atom and direct halogenation to a sp3 carbon atom and further incorporation of His (black arrow in FIG. 9) component to this sp3 carbon atom.

Example of metabolite subjected to this synthetic protocol is the metabolite nr. 242 shown in Table 1 and FIGS. 8 and 9.

1. Formation of 1-Metabolite.

The iodide halogenation of metabolite was performed via α-KG prior selective dehalogenation. First, the metabolite (100 μl of a 10 mM solution in MeOH final concentration of 1 mM) was added to 900 μl in phosphate buffer (20 mM, pH 7.8) and dehalogenase (32 μM). Reaction was allowed to proceed at 40° C. for 5 hours. Then the reaction mixture was transferred to a 1.5 ml filtration unit (3000 NMWL membrane cut-off) to remove the enzyme, and the product (mixture of partial dehalogenated products) was concentrated by liophilization. The purified intermediate (95% pure) was used for a subsequent iodization reaction as follows. The metabolite was subjected to transesterification with vinyl acetate using EL1-Lewatit. Briefly, metabolite (0.029 mmol) was dissolved in 5 mL of DMSO and 2M2B was slowly added to a final volume of 25 mL. The biocatalyst (Lewatit lipase EL1, 2.5 g) and 3 Å molecular sieves (2.5 g) were then added and the suspension maintained 30 min at 40° C. with magnetic stirring. Then, vinyl acetate (0.29 mmol) was added. When the conversion of the corresponding ester reached the maximum value, the mixture was cooled, filtered and washed with 2M2B. The product was recovered by evaporating the tert-amyl alcohol. The crude product was further purified by semi-preparative HPLC. The product so obtained was further subjected to iodide halogenation via α-KG. The halogenation reaction contained the metabolite (2.1 mM), KI (5.0 mM), and halogenase (32 μM) in phosphate buffer (20 mM, pH 7.8) plus 2 mM α-KG and up to 5% DMSO, in a final volume of 0.5 ml. After 10 h of incubation at 37° C., the reaction mixture was transferred to a 1.5 ml filtration unit (3000 NMWL membrane cut-off) to remove the enzyme, and the product was purified by semi-preparative HPLC. I-metabolite was reconstituted and diluted in DMSO and stored at −70° C. until used at a concentration of 10 μg/ml. HRMS data clearly show that the enzyme incorporated two “I” per molecule at the end of the two-step process.

2. Incorporation of Histidine.

I-metabolite was further functionalized via incorporation of a histidine tag. The incorporation of histidine was performed in the presence of the non-nucleophilic base and proton sponge 1,8-BDN as described by R. A. Kaufman et al. (loc. cit.) and F. Mazzetti and R. M. Lemmon (loc. cit.) with small modifications. Briefly, to a solution of 1-metabolite (concentration range from 0.1 to 2.3 mmol), histidine (concentration range from 0.44 to 1 mmol), and 1,8-BDN at a final concentration of 10 mM were added, in CH₃CN (10 ml final volume). The temperature was controlled at 32° C. After incubation (range from 11.52 to 424 min) the product was recovered by evaporating the CH₃CN. The corresponding product was purified by semi-preparative HPLC. The purified metabolite was found to be 98% pure.

Synthetic Method 27 for Labelling DNA 1 (5-GAC GCT GCC GAA TTC TGG CTT GCT AGG ACA TCT TTG CCC ACG TTG ACC C-3):

The substrate contain a mixture of labelled metabolites at different position of the DNA substrate (see attached figure). Only when an endonuclease cut close to the base where the Cy3 and His are close, the Cy3 is released. When attached to G, C or A the synthetic method includes a direct halogenation to a sp3 carbon atom to link it to Cy3 (grey arrow in FIG. 9) component and incorporation of His (black arrow in FIG. 9) through an NH₂ group of the metabolite.

Example of metabolite subjected to this synthetic protocol is the DNA nucleotide shown in FIG. 9.

1. Formation of 1-Metabolite.

The iodide halogenation of metabolite was performed via α-KG. The halogenation reaction contained the oligonucleotide (6.6 nmol), KI (5.0 mM), and halogenase (131 μM) in phosphate buffer (20 mM, pH 7.8) plus 2 mM α-KG, in a final volume of 0.8 ml. After 150 h of incubation at 37° C., the reaction mixture was transferred to 1.5 ml filtration unit (3-kDa membrane cut-off) to remove the enzyme, and the products were purified by semi-preparative HPLC. I-metabolite was reconstituted and diluted in DMSO and stored at −70° C. until used at a concentration of 1.5 nmol/ml. HRMS data clearly show that the enzyme incorporated one “I” per molecule per base. I-metabolite obtained is a complex mixture of halogenated molecules in which G, C, A and T bases are halogenated.

2. Incorporation of Histidine.

I-metabolite was further functionalized via incorporation of a histidine tag. A histidine molecule was incorporated by enzymatic amidation of the previous intermediate and EL1-Lewatit. Solvents were dried over 3 Å molecular sieves for 24 h prior to use. Briefly, metabolite (21.8 nmol) was dissolved in 5 mL of DMSO and 2M2B was slowly added to a final volume of 25 mL. The biocatalyst (Lewatit lipase EL1, 2.5 g) and 3 Å molecular sieves (2.5 g) were then added and the suspension maintained 30 min at 40° C. with magnetic stirring. Then, histidine (12.9 mmol) was added. An excess of histidine was used to facilitate the reaction yield. When the conversion to the corresponding ester reached the maximum value, the mixture was cooled, filtered and washed with 2M2B. The crude product was further purified by semi-preparative HPLC to obtain a mixture of his tagged oligonucleotide. Using this method histidine linking is mainly performed at the G, C and A bases due to the presence of an amine group through which the histidine is incorporated. I-metabolite was reconstituted and diluted in DMSO and stored at −70° C. until used at a concentration of 1.1 nmol/ml.

When attached to thyamine (T) the synthetic method includes a direct halogenation to sp3 carbon atoms and further incorporation of His (black arrow in FIG. 9) and Cy3 (grey arrow in FIG. 9) component to those sp3 carbon atoms.

Example of metabolite subjected to this synthetic protocol is the DNA nucleotide shown in FIG. 9.

1. Formation of 1-Metabolite.

The iodide halogenation of metabolite was performed via α-KG. The halogenation reaction contained the oligonucleotide (6.6 nmol), KI (5.0 mM), and halogenase (131 μM) in phosphate buffer (20 mM, pH 7.8) plus 2 mM α-KG, in a final volume of 0.8 ml. After 150 h of incubation at 37° C., the reaction mixture was transferred to 1.5 ml filtration unit (3-kDa membrane cut-off) to remove the enzyme, and the products were purified by semi-preparative HPLC. I-metabolite was reconstituted and diluted in DMSO and stored at −70° C. until used at a concentration of 1.5 nmol/ml. HRMS data clearly show that the enzyme incorporated one “I” per molecule per G, T and A while incorporated two “I” per thymine (T). I-metabolite obtained is a complex mixture of halogenated molecules in which G, C, A and T bases are halogenated.

2. Incorporation of Histidine.

I-metabolite was further functionalized via incorporation of a histidine tag. The incorporation of histidine was performed in the presence of the non-nucleophilic base and proton sponge 1,8-BDN as described by R. A. Kaufman et al. (loc. cit.) and F. Mazzetti and R. M. Lemmon (loc. cit.) with small modifications. Briefly, to a solution of 1-metabolite (1.3 nmol), histidine (6.8 nmol), and 1,8-BDN at a final concentration of 1 μM were added, in CH₃CN (1 ml final volume). The temperature was controlled at 32° C. After 34.1 min incubation the product was recovered by evaporating the CH₃CN. The corresponding products were purified by semi-preparative HPLC. Using this method a heterogeneous mixture of products is formed: histidine tags can be incorporated to G, A and C through one position of the ribose ring or to the two positions of T (one to the ribose and one to the base). Therefore, the product should be extensively purified by semi-preparative HPLC, to obtain the desired product, namely, that containing the His tag in the pyrimidine ring of thymine (T).

Synthetic Method 28 for Labelling DNA 1 3 (5-TGG TCA TCA GGG CTT TAC CTC CCG GAC AAT CCG GAG CTT ACG GAG TAC CTG TAG AGC TTC CTG TGC AAG C-3):

direct halogenation to a sp3 carbon atom to link it to Cy3 (grey arrow in FIG. 9) component and incorporation of His (black arrow in FIG. 9) through an NH₂ group of the metabolite. Only when an endonuclease cut close to the base where the Cy3 and His are close, the Cy3 is released. When attached to G, C or A the synthetic method includes a direct halogenation to a sp3 carbon atom to link it to Cy3 (grey arrow in FIG. 9) component and incorporation of His (black arrow in FIG. 9) through an NH₂ group of the metabolite. When attached to thyamine (T) the synthetic method includes a direct halogenation to sp3 carbon atoms and further incorporation of His (black arrow in FIG. 9) and Cy3 (grey arrow in FIG. 9) component to those sp3 carbon atoms.

The conditions for synthesis are those described in the method 27.

Synthetic Method 29 for Labelling Lambda DNA Digested with Sau3AI:

direct halogenation to a sp3 carbon atom to link it to Cy3 (grey arrow in FIG. 9) component and incorporation of His (black arrow in FIG. 9) through an NH₂ group of the metabolite (in the G base at the 5′-moiety).

DNA substrate is prepared as follows:

20 μl concentrated lambda DNA (12 μg) (from NEB)

### 7 μl Buffer NEB1 10×

### 7 μl BSA 10×

2 μl MilliQ water

### 36 μl Sau3AI 0.4 U/μl

Total reaction volume: 70 μl

Incubate 40-60 min at 37° C. Stop reactions by adding 65 mM EDTA 0.5 M pH 8 (1.5 μl for each 10 μl reaction volume) and heat the samples to 65° C. 15 min. Sample is loaded on a 20 cm long preparative gel 2% agarose, run it at 30-35 V overnight at 4° C. and cut and stain the slots with the DNA marker. Under UV light cut out the part of the gel blocks with the DNA markers in the range of ca. 100-200 bp. Cut out the desired gel region (25-40 Kb gel region) and trim excess agarose. Then proceed to the agarose gel digestion following the GELase (EPICENTRE) protocol and concentrate DNA. Once isolated the DNA then proceed as described below.

1. Formation of 1-Metabolite.

The iodide halogenation of metabolite was performed via α-KG. The halogenation reaction contained the oligonucleotide (6.6 nmol), KI (5.0 mM), and halogenase (131 μM) in phosphate buffer (20 mM, pH 7.8) plus 2 mM α-KG, in a final volume of 0.8 ml. After 150 h of incubation at 37° C., the reaction mixture was transferred to 1.5 ml filtration unit (3-kDa membrane cut-off) to remove the enzyme, and the products were purified by semi-preparative HPLC. I-metabolite was reconstituted and diluted in DMSO and stored at −70° C. until used at a concentration of 1.5 nmol/ml. HRMS data clearly show that the enzyme incorporated one “I” per molecule per G, T and A while incorporated two “I” per thymine (T). I-metabolite obtained is a complex mixture of halogenated molecules in which G, C, A and T bases are halogenated.

Steps 2 is similar to those described in synthesis 27.

Synthetic Method 30 for Labelling a Protein Substrate (Rhodonase):

the protein is partially unfolded and then a direct halogenation is performed to the valine, leucine and isoleucine amino acids to link them to Cy3 (grey arrow in FIG. 9) and His (black arrow in FIG. 9) components.

Partially unfolded rhodonase was prepared and purified as described elsewhere (Ferrer et al., Mol. Microbiol. 53, 167-182 (2005).

1. Formation of 1-Metabolite.

The iodide halogenation of protein substrate was performed via α-KG at the N-terminal position. The halogenase used in this study showed a catalytic core which was accessible to the linearized protein, namely at its N-terminal position. The halogenation reaction contained the protein (1.0 nmol), KI (5.0 mM), and halogenase (108 μM) in phosphate buffer (20 mM, pH 7.8) plus 2 mM α-KG, in a final volume of 0.5 ml. After 96 h of incubation at 37° C., the reaction mixture was transferred to a 1.5 ml filtration unit (3000 NMWL membrane cut-off) to remove the non-enzymatic reaction components, and the product was purified by semi-preparative HPLC using a Bio-Sil SEC 400 column (Bio-Rad) pre-equilibrated with phosphate buffer. Separation was performed at room temperature at a flow rate of 1 ml min¹. The following standards were used to calibrate the gel filtration column and to ensure than the protein substrate and the halogenase proteins are perfectly separated from the reaction mixture: E.GroEL (840 kDa), tyroglobulin (669 kDa), ferritin (440 kDa), gamma-globulin (158 kDa) (Ferrer et al., Mol. Microbiol. 53, 167-182 (2005). Iodide protein was reconstituted and diluted in PBS buffer and stored at −70° C. until used at a concentration of 2 pmol/ml.

2. Incorporation of Histidine.

Iodide protein was further functionalized via incorporation of a histidine tag. The incorporation of histidine was performed in the presence of the non-nucleophilic base and proton sponge 1,8-BDN as described by R. A. Kaufman et al. (loc. cit.) and F. Mazzetti and R. M. Lemmon (loc. cit.) with small modifications. Briefly, to a solution of halogenated protein (1.2 pmol), histidine (4.2 μmol), and 1,8-BDN at a final concentration of 10 mM were added, in CH₃CN (10 ml final volume). The temperature was controlled at 32° C. After 148 min incubation the product was recovered by evaporating the CH₃CN. The corresponding product was purified by fast preparative liquid chromatography (FPLC) as described elsewhere (Ferrer et al., Mol. Microbiol. 53, 167-182 (2005). Under these conditions the protein suffers an extensive denaturalization process.

To purify any of the above intermediates of final products, any of the following HPLC purification methods can be used.

### Method 1 (Standard for the Purification of the Final Labelled Metabolite)

Prontosil-AQ, 5 μm, 120 A, 250·8 mm column equipped with a Prontosil-AQ, 5 μm, 120 A, 33·8 mm pre-column, and compounds were eluted with acetonitrile (20% for 5 min, followed by linear gradients to 45% in 5 min, to 50% in 7 min and to 100% in 2 min) in triethylammonium hydrogen carbonate buffer (0.01 M, pH 8.6) at a flow of 0.4 to 3 mL/min.

### Method 2 (Standard for Medium Polar Compounds)

Analytic: on a Mediterránea 5 μM C-18 column (250×4.6 mm, Macherey-Nagel), equilibrated with 85% MeOH:15% H₂O plus 0.1% acetic acid. Column was kept at 35° C. and elution was performed at 55 atm and 0.7 ml/ml. Detection was performed both by light scattering (temp 68.2° C., 1.8 l/min) and PDA (250, 300 and 400 nm).

Semi-Preparative:

on a Mediterránea 5 μM C-18 column (250×4.6 mm, Macherey-Nagel), equilibrated with 85% MeOH:15% H₂O plus 0.1% acetic acid. Column was kept at 35° C. and elution was performed at 55 atm and 3.0 ml/ml. Detection was performed both by light scattering (temp 68.2° C., 1.8 l/min) and PDA (250, 300 and 400 nm).

### Method 3 (Standard for Medium Polar Esters)

Analytic:

using a ternary pump (model 9012, Varian) coupled to a thermostatized (25° C.) autosampler (model L-2200, VWR International). The temperature of the column was kept constant at 45° C. Detection was performed using a photodiode array detector (ProStar, Varian) in series with an evaporative light scattering detector (ELSD, model 2000ES, Alltech), and integration was carried out using the Varian Star LC workstation 6.41. For the analysis the column was a Lichrospher 100 RP8 (4.6×125 mm, 5 μm, Analisis Vinicos), and the mobile phase was 70:30 (v/v) H₂O/methanol (H₂O contained 0.1% of acetic acid) at 1 mL/min for 5 min. Then, a gradient from this mobile phase to 50:50 (v/v) H₂O/methanol was performed in 5 min, and this eluent was maintained during 15 min. For preparative scale the column was Mediterranea-C18 (4.6×150 mm, 5 μm, Teknokroma, Spain). The mobile phase was 90:10 (v/v) methanol/H₂O(H₂O contained 0.1% of formic acid) at 1.5 mL/min.

Semi-Preparative:

using a ternary pump (model 9012, Varian) coupled to a thermostatized (25° C.) autosampler (model L-2200, VWR International). The temperature of the column was kept constant at 45° C. Detection was performed using a photodiode array detector (ProStar, Varian) in series with an evaporative light scattering detector (ELSD, model 2000ES, Alltech), and integration was carried out using the Varian Star LC workstation 6.41. For preparative scale the column was Mediterranea-C18 (4.6×150 mm, 5 μm, Teknokroma, Spain). The mobile phase was 90:10 (v/v) methanol/H₂O(H₂O contained 0.1% of formic acid) at 1.5 mL/min.

### Method 4 (Standard for Short Sugars)

Analytic:

Using a pump (Spectra-Physics Inc., Model SP 8810) coupled to a Nucleosil 100-C18 column (250 mm×4.6 mm) (Sugelabor, Spain). The mobile phase was water at 0.5 ml min¹. The column was kept constant at 40° C. A differential refractometer (Waters, model 2410) was used and set to a constant temperature of 45° C.

Semi-Preparative 1:

using a system equipped with a Waters Delta 600 pump, a Nucleosil 100-C18 column (250 mm×10 mm) (Sugelabor, Spain) coupled to a precolumn (50 mm×10 mm) packed with the same stationary phase. A differential refractometer (Varian, model 9040) set to 35° C. and a fraction collector (Waters) were used. Water was the mobile phase (2.4 ml min¹), and the column temperature was kept constant at 40° C.

Semi-Preparative 2:

with a quaternary pump (Delta 600, Waters) coupled to a 5 μM Lichrosorb-NH₂ column (4.6 mm×250 mm) (Merck). Detection was performed using an evaporative light scattering detector DDL-31 (Eurosep) equilibrated at 85 C. Acetonitrile:water 85:15 (v/v), degassed with helium, was used as mobile phase at 0.9 ml min¹ for 8.1 min. Then, a gradient from this eluent to acetonitrile:water 75:25 (v/v) was performed in 2 min, and held for 6 min. A new gradient to acetonitrile:water 70:30 (v/v) was performed in 5 min and held for 14 min. Total analysis time was 35 min. The column temperature was kept constant at 25° C.

### Method 5 (Standard for Short Length Esters)

Analytic:

The pump (Spectra-Physics Inc., Model SP 8810) was coupled to a Nucleosil 100-C18 column (250 mm×4.6 mm) (Sugelabor, Spain). The mobile phase was 80% MeOH:20% H₂O at 0.5 ml min¹. The column was kept constant at 40° C. A differential refractometer (Waters, model 2410) was used and set to a constant temperature of 45° C.

Semi-Preparative:

using a system equipped with a Waters Delta 600 pump, a Nucleosil 100-C18 column (250 mm×10 mm) (Sugelabor, Spain) coupled to a precolumn (50 mm×10 mm) packed with the same stationary phase. A differential refractometer (Varian, model 9040) set to 35° C. and a fraction collector (Waters) were used. 80% MeOH:20% H₂O was the mobile phase (2.4 ml min¹), and the column temperature was kept constant at 40° C.

Method 6 (Standard for Long Length Fatty and their Sugar Derivatives)

Analytic:

using a system equipped with a Spectra-Physics pump, a Sugelabor Nucleosil 100-C18 column (250×4.6 mm) and a refraction index detector (Spectra-Physics). For the analysis methanol/water 95:5 (v/v) was used as mobile phase (flow rate 1.5 mL/min) and the temperature of the column was kept constant at 40° C.

Semi-Preparative:

using a system equipped with a Waters Delta 600 pump, a Nucleosil 100-C18 column (250 mm×10 mm) (Sugelabor, Spain) coupled to a precolumn (50 mm×10 mm) packed with the same stationary phase. A differential refractometer (Varian, model 9040) set to 35° C. and a fraction collector (Waters) were used. 90% MeOH:10% H₂O or 95% MeOH:5% H₂O was the mobile phase (2.8 ml min¹), and the column temperature was kept constant at 40° C.

### Method 7 (Standard for Folate Derivatives)

Analytic:

on a Nucleosil 100-C18 column (250×4.6 mm) using isocratic program of 88% A and 12% B in 30 min at a flow rate of 0.3 ml/min. Mobile phase A consisted of 0.1% formic acid while mobile phase B consisted of 0.1% formic acid in a 95:5 acetonitrile/water solution. Diode array detector (DAD) was set at 290 nm.

Semi-Preparative:

using a system equipped with a Waters Delta 600 pump, a Nucleosil 100-C18 column (250 mm×10 mm) (Sugelabor, Spain) coupled to a precolumn (50 mm×10 mm) packed with the same stationary phase. A differential refractometer (Varian, model 9040) set to 35° C. and a fraction collector (Waters) were used. 80% MeOH:20% H₂O was the mobile phase (2.4 ml min¹), and the column temperature was kept constant at 40° C.

### Method 8 (Standard for Short Length Alkanes)

Analytic:

on a 5 μM LiChroCart 125-4 RP 18 column (Merck). The initial solvent composition was 20% methanol-80% phosphate buffer (20 mM), pH 4.8, reaching 100% methanol within 14 min at a flow rate of 1 ml min⁻¹. Detection was performed both by light scattering (temp 68.2° C., 1.8 l/min).

Semi-Preparative:

on a 5 μM LiChroCart 125-4 RP 18 column (Merck) at a flow rate of 1 ml min⁻¹. For sufficient separation, the solvent system, consisting of methanol (eluent A) and ammonium acetate buffer (20 mM, pH 4.8) (eluent B), was started in a ratio of 20% A and 80% B and reached 70% A and 30% B within 12.5 min, and then it was changed to 100% A within 30 s and held constant for another min.

### Method 9 (Standard for Medium and Long Length Alkanes)

Analytic:

on a 2.7 μM Halo C8 (150×4.6 mm) column with MeOH:H₂O:acetic acid (750:250:4) as buffer A and acetonitrile:methanol:THF:acetic acid (500:375:125:4) as buffer B at a flow rate of 0.8 ml min⁻¹. Gradient was performed from 100 buffer A to 100% buffer B in 60 min. Column was kept at 35° C. and elution was performed at 55 atm and 0.8 ml/ml. Detection was performed both by light scattering (temp 68.2° C., 1.8 l/min).

Semi-Preparative:

on a 2.7 μM Halo C8 (150×4.6 mm) column with MeOH:H₂O:acetic acid (750:250:4) as buffer A and acetonitrile:methanol:THF:acetic acid (500:375:125:4) as buffer B and at a flow rate of 2.2 ml min⁻¹. Gradient was performed from 100 buffer A to 100% buffer B in 60 min. Column was kept at 35° C. and elution was performed at 55 atm and 0.8 ml/ml. Detection was performed both by light scattering (temp 68.2° C., 1.8 l/min).

### Method 10

Analytic 1:

on a Nucleosil 100-C18 column (250×4.6 mm) using isocratic program of 88% A and 12% B in 30 min at a flow rate of 0.3 ml/min. Mobile phase A consisted of 0.1% formic acid while mobile phase B consisted of 0.1% formic acid in a 95:5 acetonitrile/water solution. Diode array detector (DAD) was set at 290 nm.

Analytic 1:

on a SC125/Lichrospher column (250×4.6 mm). The mobile phase was 0.01% (vol/vol) H₃PO₄ (87%) and 50% (vol/vol) methanol at 1.0 ml min'. The column was kept constant at 40° C. A differential refractometer (Waters, model 2410) was used and set to a constant temperature of 35° C.

Semi-Preparative:

using a system equipped with a Waters Delta 600 pump, a Nucleosil 100-C18 column (250 mm×10 mm) (Sugelabor, Spain) coupled to a precolumn (50 mm×10 mm) packed with the same stationary phase. A differential refractometer (Varian, model 9040) set to 35° C. and a fraction collector (Waters) were used. 0.01% (vol/vol) H₃PO₄ (87%) and 50% (vol/vol) methanol was the mobile phase (2.0 ml min¹), and the column temperature was kept constant at 40° C.

### 8. Incorporation of (H) derivatives to the poly(A)-nitrilotriacetic-Co(II) complex

The final step in the labeled metabolite development is the incubation of the histidine functionalized Cy3-metabolites with poly(A)-nitrilotriacetic-Co(II) complexes in 50 mM phosphate buffer, 50 mM NaCl, pH 7.5 for 1 hour at 25° C. When required, DMSO was added to increase substrate solubility up to 50%. Briefly, 0.015 mmol (13 mg) (H) was dissolved in 5 ml 50 mM phosphate buffer, 150 mM NaCl, pH 7.5 (PBS), containing up to 50% DMSO depending on the solubility of the molecule, with 0.025 mmol (B) in a 15 ml falcon tube which was placed on a rotatory shaker for 1 h at 25° C. To ensure that each (H) molecule binds to (B) through both of its His residues, analytical HPLC and ⁵⁹Co-NMR analyses were performed (see data in Table S1), and only derivatives incorporating single Cy3-labelled molecules (I) were purified by semipreparative reverse-phase HPLC (Prontosil-AQ, 5 μm, 120 A, 250·8 mm column equipped with a Prontosil-AQ, 5 μm, 120 A, 33·8 mm pre-column, and compounds were eluted with acetonitrile (20% for 5 min, followed by linear gradients to 45% in 5 min, to 50% in 7 min and to 100% in 2 min) in triethylammonium hydrogen carbonate buffer (0.01 M, pH 8.6) at a flow of 0.4 to 3 mL/min. Overall, yields higher than 93% were achieved. Fractions containing labelled metabolites were pooled and solvent evaporated. Molecules were dissolved in PBS buffer containing 50% DMSO and stored in 384 microtiter plates at −70° C. until used at concentration of 40 μM.

### Reaction Scheme (a General Schematic Metabolite “M” is Represented)*:

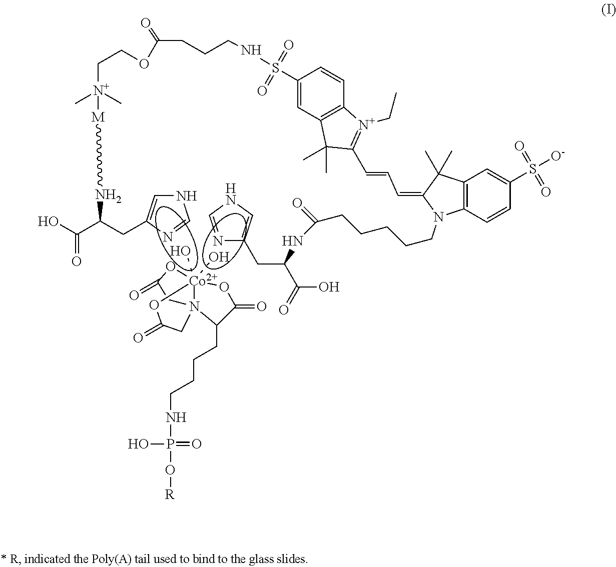

### 9. Binding of (H) to gold nanoparticles

Au-6,8-dithioctic acid (TA) clusters were synthesized as described by Abad et al., J Am Chem Soc 127, 5689 (2005) and used to create Au-TA-ANTA-Co(II)-metabolite-Cy3 clusters. The Au-TA clusters were linked to (H) by overnight amidation in a single step in the presence of 3 mM N-hydroxysuccinimide (NHS, Fluka) and 3 mM 1-ethyl-3-[3-(dimethylamino)propyl]carbodiimide (EDC, Sigma) in 20 mM HEPES buffer (pH 7.5). Further purification of nanoparticles (J) containing Cy3 labeled metabolites was carried out by ultrafiltration through low-adsorption hydrophilic 30000 NMWL cutoff membranes (regenerated cellulose, Amicon). As an average value, a concentration of 9×10¹⁰ particles/ml of diameter ˜2.9±0.8 nm that corresponds to a surface area of ˜141±3 cm²/ml, binds 62.5 pmol of (H).

### Reaction Scheme (a General Schematic Metabolite “M” is Represented):

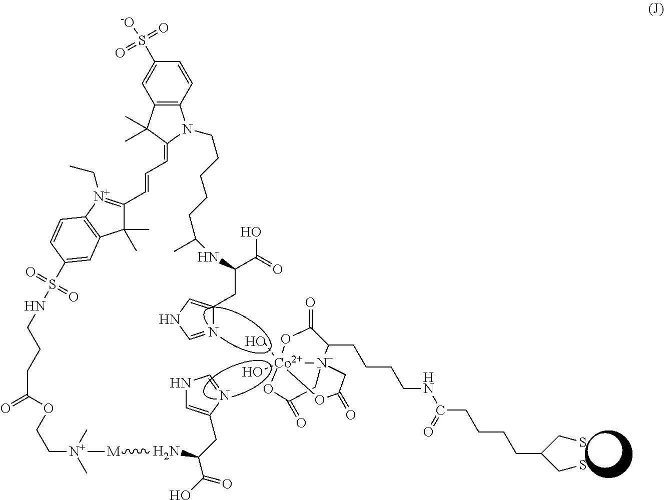

### Synthesis Example 1

Step (1) to (5) are described above.

Step 6. X-Gal Source

5-Bromo-4-chloro-3-indolyl β-D-galactopyranoside (X-Gal) was provided by Roche Diagnostics (1N, USA) (ref. 10651745001), further reconstituted and diluted in DMSO, and stored at −70° C. until used at a concentration of 100 mg/ml.

Step 7. Formation of Iodide X-Gal, Further Incorporation of Histidine to Iodide-X-Gal and Formation of Cy3-Labeled X-Gal

The iodide halogenation of X-Gal was performed via I⁻ as follows. The reaction mixture was incubated at 37° C. with X-Gal (0.080 mmol; 32.69 mg dissolved in 0.1 ml DMSO), KI (75 mM), 2 mM NADH, and REBr dehalogenase (4 mg/ml, 100 μL) in phosphate buffer (20 mM, pH 7.8) at a final volume of 0.5 ml. The final volume of DMSO was keep at 20% v/v in order to maintain the solubility of the X-Gal. After 24 hour of incubation reaction product was separated by HPLC on a Hypersil 5 μM C-18 column (250×4.6 mm, Macherey-Nagel) equilibrated with KH₂PO₄ (50 mM) and acetonitrile (85:15 v/v). Runs were performed by gradient elution from a starting mobile phase of KH₂PO₄ (50 mM) and acetonitrile (85:15 v/v) to a final mobile phase consisting of KH₂PO₄ (50 mM) and acetonitrile (60:40 v/v). The purified iodide X-Gal was found to be 95% pure (13.8 g, 26% yield; white solid crystalline powder). Iodide X-Gal was reconstituted and diluted in DMSO and stored at −70° C. until used at a concentration of 10 mg/ml. HRMS data clearly show that the enzyme incorporated two “I” per molecule. HRMS: calculated for C₁₄H₁₃BrClI₂NO₆, 658.7704, [M⁺H⁺]. found was 659.7770.

### Reaction Scheme:

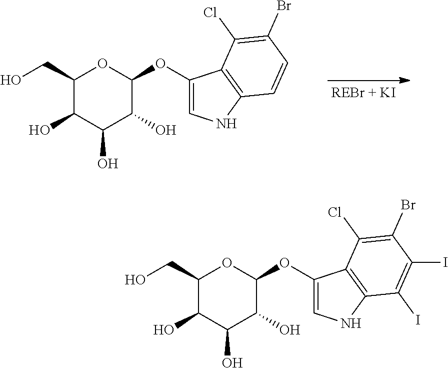

Iodide X-Gal was further functionalized via incorporation of a histidine tag. The incorporation of histidine was performed in the presence of the non-nucleophilic base and proton sponge 1,8-bis-(dimethylamino)-napthalene (Sigma) as described by R. A. Kaufman et al. (loc. cit.) and F. Mazzetti and R. M. Lemmon (loc. cit.) with small modifications. Briefly, to a solution of iodide X-Gal (0.1 mmol; 65.8 mg), histidine (1 mmol, 156 mg), and 1,8-bis-(dimethylamino)-napthalene at a final concentration of 10 mM, in CH₃CN. The temperature was controlled at 32° C. After 100 min incubation the product was recovered by evaporating the CH₃CN. The corresponding product was purified by semipreparative reverse-phase HPLC (Prontosil-AQ, 5 μm, 120 A, 250·8 mm column equipped with a Prontosil-AQ, 5 μm, 120 A, 33·8 mm pre-column, and compounds were eluted with acetonitrile (20% for 5 min, followed by linear gradients to 45% in 5 min, to 50% in 7 min and to 100% in 2 min) in triethylammonium hydrogen carbonate buffer (0.01 M, pH 8.6) at a flow of 0.4 to 3 mL/min. The purified metabolite was found to be 98% pure (21 mg, 32% yield; white solid crystalline powder). Mass spectrometry was used to confirm the structure. HRMS: calculated for C₂₀H₂₁BrClIN₄O₈ was 685.9276, [M⁺H⁺]. found 686.9266. As shown, the result was within of the calculated molecular mass.

### Reaction Scheme:

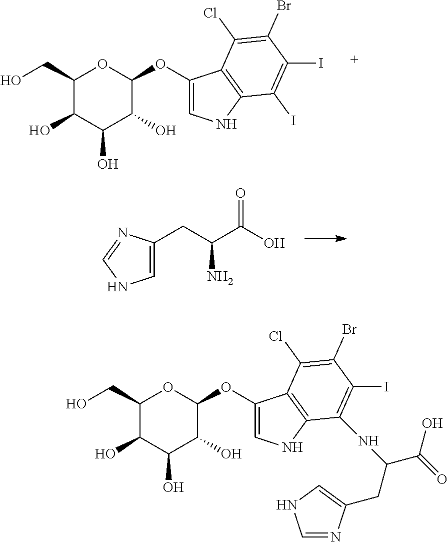

The corresponding labeled quaternary ammonium X-Gal were obtained in the presence of 1,8-bis-(dimethylamino)-napthalene (Sigma) as described by R. A. Kaufman et al. (loc. cit.) and F. Mazzetti and R. M. Lemmon (loc. cit.) with small modifications. Briefly, the general method for the synthesis of quaternary amines is as follows. Reaction mixture (2 ml) contains histidine tagged iodide-X-Gal (0.078 mmol, 53.4 mg), 0.78 mmol of (E) and 1,8-bis-(dimethylamino)-napthalene at a final concentration of 100 mM in DMSO. The temperature was controlled at 32° C. After 100 min incubation the product was recovered by evaporating the CH₃CN. The labeled product was purified by semipreparative reverse-phase HPLC (Prontosil-AQ, 5 μm, 120 A, 250·8 mm column equipped with a Prontosil-AQ, 5 μm, 120 A, 33·8 mm pre-column, and compounds were eluted with acetonitrile (20% for 5 min, followed by linear gradients to 45% in 5 min, to 50% in 7 min and to 100% in 2 min) in triethylammonium hydrogen carbonate buffer (0.01 M, pH 8.6) at a flow of 0.4 to 3 mL/min. The purified metabolite was found to be 98% pure (47.3 mg, 41% yield; white solid crystalline powder). Mass spectrometry was used to confirm the structure. HRMS: calculated for C₆₅H₈₂BrClN₁₁O₁₈S₂ was 1482.4153, [M^{+H}⁺]. found 1483.4106.

### Reaction Scheme

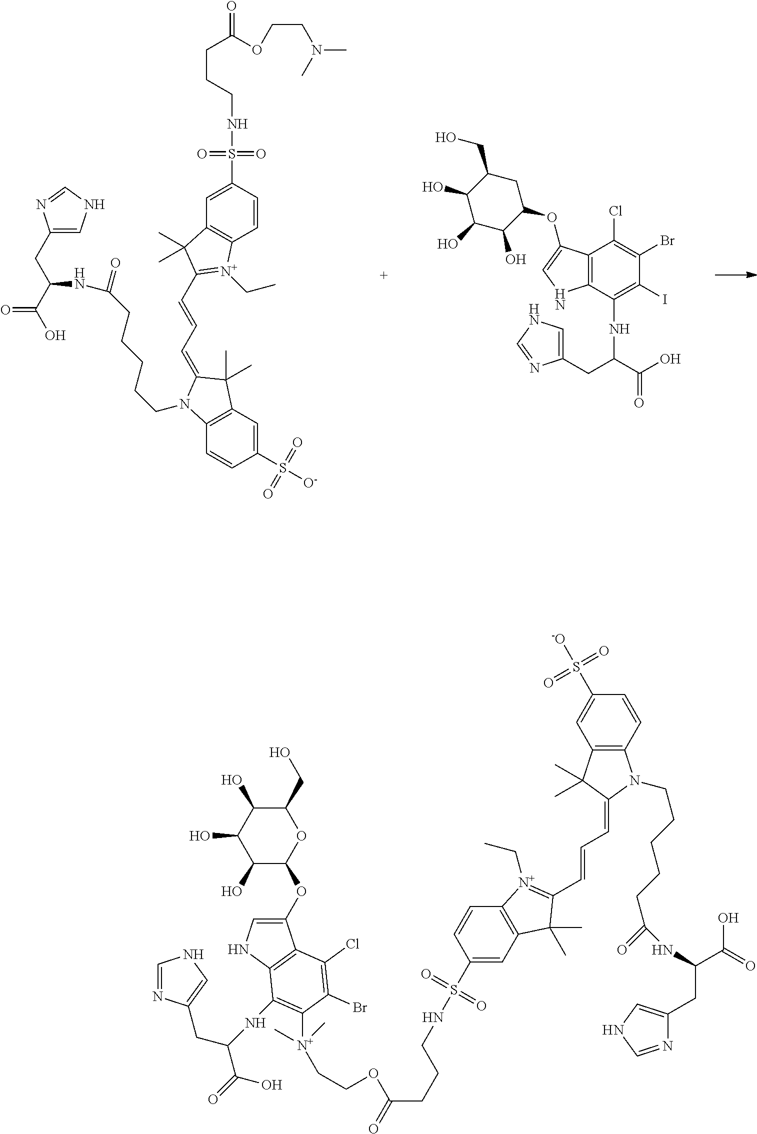

Steps (8) and (9) are as described above.

### Example 6

SM Spotting and Detection of Protein-SM Transformations.

SM-Cy3s (0.25 nL droplets of 3.5 pmol/l in DMSO/PBS=1:1; spot size 400 μm diameter) were spotted by means of a MicroGrid II micro-arrayer (Biorobotics) onto a glass slide, followed by fixation by cross-linking through the poly A tail. Each SM was spotted in triplicate on each slide. Buffer controls were applied for comparison. Sixty μl of cell lysate at a protein concentration of 0.1 mg/ml in PBS buffer was deposited on the slide and incubated at room temperature for 30 to 180 min. PBS buffer was used as a control. The slide was then washed with PBS and deionized water, dried by standard array slide centrifugation protocol and fluorescence intensities of the spots were measured with a microarray laser scanning system (Axon) set to a pmt of 500 and 100% laser power. Signals were analyzed and quantified using GenePix pro 4.1 software (Axon). Through the analysis of three micro-arrays of three biological replicates, average values and standard deviations were calculated using the Microsoft Excel programme. The average deviation is given in the caption of Table 3. Each data point was normalized to the signal intensity obtained with X-Gal-Cy3 and pure β-galactosidase (150 pg/ml), and normalized signal intensities were compared to those produced by the control array incubated with buffer. Normalization with the Cy3 β-Gal-signal intensity eliminated errors of signal intensity variation between arrays. All values given in Table 3 are therefore Cy3-signal corrected signal intensities after lysate incubation compared to buffer-only incubation. On the average of four Cy3-signal intensity measurements, the signal intensity of SM-Cy3 on the buffer-incubated array was 0.2% lower than the signal intensity of lysate-incubated array, showing high reproducibility of the Cy3-signal intensity and thus the legitimacy of using it as standardization factor.

### Example 7

Data Analysis.

After background subtraction, signal intensities for each replica were normalized and manually analyzed using Excel program (Microsoft) and GenePix Pro 6 Demo Program (http://www.moleculardevices.com/product literature/family links.php?familyid=14). MultiExperiment Viewer software (Sun Microsystems Inc) was used to visualize and compare differences in signal intensity.

### Example 8

Construction of (Meta)Genomic Libraries.

DNA was extracted from three environments which differdiffering by in regard to the species composition and richness and main environmental constraints and the corresponding insert-libraries were constructed.

Kolguev Island Coastal Seawater (KOL):

A 200 ml sample of the coastal seawater of Kolguev Island (Barents Sea, Russia) was placed into a 1 L Erlenmeyer flask containing sterile crude oil (Arabian light, 0.5% (vol/vol)) and nutrients ([NH_{4]}₂PO₄, 0.05% (w/vol)). After four weeks of incubation at 4° C. on a rotary shaker, total DNA was extracted the culture with G'NOME DNA Extraction Kit (Qbiogene; Carlsbad, Calif.), and a metagenome expression library in the bacteriophage lambda-based ZAP phagemid vector (ZAP Express Kit, Stratagene), was constructed as described in the manufacturers' protocols. A library of 8×10⁶ phage particles, average insert size about 6 kbp, was thereby generated. Phage particles were used to infect E. coli XL1 Blue MRF′ and subsequent mass excision was performed by using E. coli XLOLR cells, as recommended by the supplier. Vulcano Island (VUL): The fosmid library was established from the DNA isolated from the enrichment of microbial community from acidic pool of Porto Levante on Vulcano Island, Italy. Sulphfur and iron-containing sandy volcanic acidic (pH 1.5-4) hydrothermal pool sample was enriched with the medium 874 (pH 1.7) (DSMZ, http://www.dsmz.de) containing 0.1% (w/vol) yeast extract and was incubated for 4 weeks at 45° C. with shaking. The total amount of fosmid clones was 11520. The DNA was extracted using G'NOME DNA Extraction Kit (BIO101, Qbiogene) and was cloned using Fosmid Library Production Kit (Epicentre) as recommended by the suppliers.

L'Atalante Seawater-Brine Interface Sample (L'A):

The brine-seawater interface above hypersaline anoxic basin L'Atalante (Eastern Mediterranean Sea) was sampled during the MedBio2 oceanographic cruise in December 2006 from the depth of 3.431 meters. The 50 mL-samples were placed into sterile 100 ml Hungate bottles containing resazurine (anoxia indicator), 1 g/L yeast extract and 2 mM ¹³C-glucose. The salinity measured immediately after the cast was 180 g/L (NaCl). After six months of incubation at 14° C. in the dark, the total DNA was extracted with G'NOME DNA Extraction Kit (BIO101, Qbiogene) and was further cloned using Fosmid Library Production Kit (Epicentre) as recommended by the suppliers.

P. putida KT2440: the total DNA was extracted with GNOME DNA Extraction Kit (BIO101, Qbiogene) from 100 ml culture of this bacterium grown as described above and was further cloned using Fosmid Library Production Kit (Epicentre) as recommended by the suppliers.

### Example 9

Culture Conditions and General Procedure for the Preparation of Protein Lysates.

Cells of P. putida KT2440 were grown at 30° C. in 100 mL-flasks filled with 10 mL minimal medium (MM) prepared as follows. A solution with “Epure” water containing (NH₄)₂SO₄ (2 g/L), Na₂HPO₄12H₂0 (6 g/L), KH₂PO₄ (3 g/L) and NaCl (3 g/L) was adjusted to pH 7.0±0.2 and then autoclaved. The medium was supplemented with 20 mM MgSO₄, 10 mM FeSO₄ and 15 mM Na-succinate (from a stock solution sterilized by filtration through a 0.22 μm filter (Millipore)). Like in case of L'A and Volcano libraries, the individual clones were incubated overnight without shaking at 37° C. in LB medium, 12.5 g/ml chloramphenicol, in 384 microtiter plates. An aliquot of 10 μl each culture were pooled together in appropriate flasks filled with 1L medium and subsequently incubated 6 additional hours (OD_{600 nm} 1.5) at 37° C. For lambda phage Kolguev library, mass excision in E. coli XLOLR was done following the protocol recommended by the supplier. The pool of clones (from phagemids of fosmids) was grown with shaking at 37° C. in LB medium, with 50 g/ml kanamycin until OD_{600 nm} 1.5. After cultivation the cells were harvested by centrifugation (5000 g) for 15 min to yield 2-3 g/L of pellet. The cell pellet was frozen at −80° C. overnight and then thawed. Cold PBS buffer was added directly to the frozen pellets (1.2 ml per 0.3 grs cell pellet). The mixture was vortexed to homogeneity and subsequently sonicated for 2 min (total time). The extract was centrifuged for 15 min at 15,000 g to separate cell debris and intact cells. The supernatant was carefully collected avoiding disturbing the pellet, and transferred to new tubes. Then, the extracts were immersed in liquid nitrogen and subjected to lyophilisation in order to avoid volatile contaminants. After that, extracts were resuspended in 1.2 mL PBS buffer and protein concentration was determined by a standard procedure (38) and further fixed to 0.1 mg/ml.

### Example 10

Representative Procedure for the Synthesis of SMs-Cy3 Gold Nanoparticles for Identification and N-Terminal Sequencing of SM-Acting Proteins.

Au-6,8-dithioctic acid (TA) clusters were synthesized as described by Abad et al. (36) and used to create Au-TA-ANTA-Co(II)-SMs-Cy3 clusters. Briefly, the Au-TA clusters were incorporated to the ANTA-Co(II)-SMs-Cy3 by overnight amidation in a single step in the presence of 3 mM N-hydroxysuccinimide (NHS, Fluka) and 3 mM 1-ethyl-3-[3-(dimethylamino)propyl]carbodiimide (EDC, Sigma) in 20 mM HEPES buffer (pH 7.5).13 Further purification was carried out by ultrafiltration through low-adsorption hydrophilic 30 000 NMWL cutoff membranes (regenerated cellulose, Amicon). Enzymatic binding was carried out by incubation of the functionalized nanoparticles (40 g cm⁻³) in PBS buffer, pH 7.5 overnight at room temperature with protein solution (0.1 mg/ml in PBS). Au-TA-ANTA-Co2+-protein nanoparticles were separated from unbound enzyme molecules by ultrafiltration through low-adsorption hydrophilic 100 000 NMWL cutoff membranes (Amicon). Identification of bound proteins was performed by in situ trypsin digestion and ESI-Q-TOF MS/MS mapping (M. Ferrer et al., Nature 445, 91 (2007)) that provides improved mass measurement accuracy for peptide sequencing and enables unambiguous protein identification. ESI-Q-TOF MS/MS analyses were performed at the Sequencing Core Facility of the Autonomous University of Madrid. For each experiment, up to three binding experiments were prepared and analyzed.

### Example 11

PCR Amplification of Genes Encoding Hypothetical Proteins from P. putida KT2440 and Metagenomic Libraries.

P. putida KT2440 and metagenomic hypothetical proteins were amplified by PCR from genomic and metagenomic DNA using the set of primers detailed in Table 4. Cycling parameters were 2 min at 95° C. followed by 25 cycles at 95° C. for 30 s, 66° C. for 30 s, and 72° C. for 20-140 s (see specific elongation temperature for each protein in Table 4), and ending with 10 min at 72° C. KOL-1, -2 and -7 and VUL-9 proteins were amplified by PCR from the corresponding metagenomic DNA library using the set of primers detailed in Table 4. Cycling parameters were 5 min at 95° C. followed by 25 cycles at 95° C. for 1 min, 55° C. for 1 min, and 72° C. for 1 min, and ending with 7 min at 72° C. PCR products were ligated in pGEMT plasmid (Promega).

### Example 12

Gene Cloning, Expression and Purification.

All proteins characterized in this study were cloned into pET-41 Ek/LIC vector (Novagen) and expressed with an N-terminal fusion to 6×His tag, according to manufacturer's instructions and plasmids were subsequently isolated and introduced into E. coli ORIGAMI(DE3) pLysS expression host. Full-length of gene coding for proteins were amplified from the P. putida genome by polymerase chain reaction (PCR), whereas full-length of proteins from libraries were amplified with degenerated primers based on the Q-TOF peptide obtained (Table 4). Proteins were expressed in Escherichia coli strain ORIGAMI(DE3) pLysS (Novagen). Cultures were grown overnight in Luria-Bertani medium containing 100 mg/ml ampicillin and 50 mg/ml kanamycin, then diluted 1:100 in fresh medium. Cells were grown at 37° C. to a final OD₆₀₀ of 0.5, induced with 1 mM isopropyl-D-thiogalactopyranoside (IPTG) and incubated at 37° C. for an additional 4 h. Cell pellets were collected by centrifugation at 4 1C for 20 min at 8,000 g. Pellets were then suspended in 25 ml prechilled lysis buffer (Complete EDTA-free protease inhibitor tablet (Roche), 150 mM NaCl, 1 mM dithiothreitol, 50 mM Hepes, pH 7.0, 5 mM imidazole) and lysed by sonication on ice for 3 min with 30 intervals. Cell lysates were centrifuged once more at 4° C. for 20 min at 30,000 g, and the soluble fractions were retained. Proteins were purified using a 1-ml HisTag (Novagen) according to the manufacturer's protocols and eluted in 250 mM imidazole and 50 mM HEPES, pH 7.0. Elutions of 500 l were collected, pooled and concentrated 100-fold using Microcon YM-3 spin columns (Millipore). The molarities of all purified proteins were determined by using the corresponding extinction coefficient. Purified proteins were stored at −80° C. until further use.

### Example 13

Receiver Operating Characteristic (ROC) Analysis.

The “receiver operating characteristic” (ROC) analysis is used to evaluate the performance of a binary classifier separating two populations (positive and negative cases) and it is becoming a standard in evaluating and comparing prediction methods (T. Fawcett, Pattern Recogn Lett 27, 861 (2006)). This analysis can be applied to classifiers (prediction methods) which associate a numerical score to each case. Ideally the classifier would tend to associate high scores to the positive cases and low scores to the negative ones (or the other way around). The analysis is performed by sorting all the cases (positives and negatives) by their associated scores. This sorted list is then cut at different score thresholds and for each cut the true and false positive rates (TPR and FPR) are calculated as

TPR=TP/(TP+FN)

FPR=1−(TN/(TN+FP))

Where, TP: “true positives”—cases predicted as positives (above the threshold for that particular cut) which are in fact positives (correct predictions); FN: “false negatives”—cases predicted as negatives (below the threshold) which are actually positives (wrong predictions); TN: “true negatives”; FP: “false positives”. “TP/(TP+FN) is also known as “sensitivity” and gives an idea of the fraction of positives recovered at this particular cut of the list (note that TP+FN is the total number of positive cases). “TN/(TN+FP)” is also known as “specificity”.

The ROC plot is constructed by plotting TPR against FPR for the different cuts of the list. In such a representation, a random method (a classifier without discriminative power which spreads positives and negatives uniformly across the sorted list of scores) will produce a diagonal line from (0,0) to (1,1). Methods with some discriminative power will generate curves above this line. The higher the curve (closer to the (0,1) corner) the better the method. A method with a perfect discriminative power (which puts all the positives together at the top of the list, associated to the highest scores) would be represented by a single point at (0,1). A parameter based in this representation which quantifies the global performance of a method in the AUC (“area under curve”). The random method commented above will produce an AUC value of 0.50, while discriminative methods will produce AUC values between 0.50 and 1.00 (perfect discrimination).

In this study case, we used this analysis for evaluating the ability of our array to discriminate compounds which are actually metabolized by P. putida from those which are not. The score is the fluorescence intensity, under the idea that there will be a positive relationship between the intensity in the array and the compound being metabolized in P putida. We assume that compounds metabolized by P. putida are those which act as substrates in one or more chemical reactions in the metabolism of P. putida as reconstructed from KEGG (cf. above) for that organism. We took as P. putida reactions those for which the EC code or the KEGG orthologs code (ko) are associated to a P. putida K2440 gene (“PPU” in KEGG nomenclature).

### Example 14

Calculation of Z-Scores for Comparing Samples in Terms of Functional Classes.

The z-score (Zᵢ) for a given intensity value (Iᵢ) is calculated as:

$${Z}_{i} = \frac{{I}_{i} - \overline{I}}{σ}$$

Where Ī and σ are the average and standard deviation of the all the intensity values in the array respectively. A Zi value of 0 means that the intensity is right in the average. A positive value means that the intensity is higher than the average, and the other way around for negative values. For each KEGG functional class for which more than 1676 compounds are in the array, we calculate the average Zᵢ value of all the compounds belonging to that class ( Z̅ᵢ̅). A high Z̅ᵢ̅ value indicates that this class of compounds is highly metabolized in that particular sample (array). Comparing the Z̅ᵢ̅ values for a given class in two arrays (samples) it is possible to known which metabolic activities are “emphasized” or “repressed” from one condition to the other. To quantify that, for each class we calculate the difference of its Z̅ᵢ̅ values in the two samples

(Δ Z̅ᵢ̅= Z̅ᵢ̅₂̅− Z̅ᵢ̅₁̅).

It is important to note that the results in terms of which classes are over/under expressed are exactly the same using Zᵢ or Iᵢ values since, by definition, they are correlative (see equation above). We used Zi values simply because they are easier to interpret in terms of relative intensities.

### Example 15

Hydrogenase Activity and IR Measurements.

The oxidation of H₂ (H₂ uptake) was followed spectrophotometrically due to the reduction of methyl viologen (MV) as described by De Lacey et al., J Biol Anorg Chem 9, 636 (2004):

H₂2e⁻+2H⁺+2BV²⁺2BV⁺

Buffers:

The buffers used for the activity assay were: 1 mM MV in Tris-HCl 20 mM buffer pH 8.1 and sodium dithionite 10 or 100 mM in Tris-HCl 20 mM buffer pH 8.1.

Sample preparation: 0.2 mg/mL L'A62 protein in buffer Tris-HCl 20 mM pH 8.1. To this solution 1 μL of dithionite 10 mM solution is added.

The IR spectra was measured as described by De Lacey et al. using a protein solution of 12 mM in Tris-HCl 50 mM pH 8.1.

### Example 16

Construction rRNA Gene Clone Libraries and Clone Sequencing.

PCR amplification was performed with 0.1 ng DNA template. 16S rRNA genes were amplified using the Eubacteria-specific forward primer F27 (5′-AGAGTTTGATCMTGGCTCAG-3′) in case of KOL and L'A and a universal F530 primer (5′-TCCGTGCCAGCAGCCGCCG-3′) for VUL library, in all cases in the combination with the universal reverse primer R1492 (5′-CGGYTACCTTGTTACGACTT-3′). Amplification was done in 20 μl reaction volume with recombinant Taq DNA Polymerase (Invitrogen, Germany) and original reagents, according to the basic PCR protocol, with an annealing temperature of 45° C. (VUL) and 50° C. C (L'A and KOL), for 30 cycles. PCR amplicons were purified by electrophoresis on 0.8% agarose gels, followed by isolation from excised bands using a QIAEX II Gel Extraction Kit (Qiagen, Germany). The purified PCR products were ligated into plasmid vector pCRII-TOPO (TOPO TA Cloning kit, Invitrogen, Germany) with subsequent transformation into electrocompetent cells of E. coli (TOP 10) (Invitrogen, Germany). After blue/white screening, randomly picked clones were resuspended in PCR-lysis solution A without proteinase K (67 mM Tris-Cl (pH 8.8); 16 mM NH₄SO₄; 5 M-mercaptoethanol; 6.7 mM MgCl 2; 6.7 M EDTA (pH 8.0) (Sambrook and Russel, 2002) and heated at 95 C C for 5 min. The lysate (1 μl) was used as DNA template for PCR amplification using primers M13F (5′-GACGTTGTAAAACGACGGCCAG-3′) and M13R (5′-GAGGAAACAGCTATGACCATG-3′). After verification on the agarose gel, PCR products were purified with MinElute 96 UF PCR purification kit (Qiagen, Germany) and sequenced with M13 and M13R primers according to the protocol for BigDye Terminator v1.1 Cycle Sequencing Kit from Applied Biosystems (USA).

### Example 17

Dose-Response Curves Determined with E. coli β-Galactosidase (β-Gal).

To prove that the present array can discern active and non-active proteins and in parallel to determine the sensitivity of the system, we determined the molecule dose-response behaviour of active and inactive -Gal and vice versa. 5-Bromo-4-chloro-3-indolyl-D-galactopyranoside (X-Gal) modified with Cy3 as described in SYNTHESIS EXAMPLE 1, was used as substrate. FIG. 4 shows the X-Gal-associated pixel intensity from the scanned slides plotted against the amount of X-Gal. As shown, 30 min incubation with a solution of only 5 ng pure β-Gal ml⁻¹ at 20° C. was sufficient to ensure 50% of maximum fluorescence (F₅₀) when 0.25 nl of a solution containing 0.12 pmol ml⁻¹ substrate was spotted (signal to noise (S/N) ratio above 71), while inactive protein was unable to release the Cy3 dye. Further, using 2.52 pmol X-Gal-Cy3 ml⁻¹ 50% of maximum fluorescence was reached above 1.86 ng β-Gal ml⁻¹ (signal saturation above 95 ng ml⁻¹). According to this data, micro-array analysis were further performed by spotting 0.25 nl of substrate solutions at concentration of 2.52 pmol ml⁻¹ and by arraying the slides with at least 0.10 mg protein lysate ml⁻¹ to ensure detection of all proteins in the extract (it should be noted that enzyme concentrations as low as 1.5 ng ml⁻¹ were sufficient to ensure appropriate detection with a S/N 10).

### Example 18

Characteristics of Microbial Communities Examined.

In order to assess the utility of the array for the analysis of complex microbial communities as represented in metagenomic libraries, we obtained samples from three habitats with distinct physico-chemical characteristics and therefore distinct microbial communities and metabolic characteristics. The general characteristics of the three habitats are as follows: (i) acidic (pH 1.0-3) sulfur- and iron-rich (from 3 to more than 500 mg/l) sediments of a hydrothermal pool (25-75° C.) of Porto Di Levante, Vulcano Island (Italy) (ii) oil-polluted, cold (1° C.) coastal seawater sampled near Kolguev Island, Barents Sea, Russia, and (iii) the seawater-brine interface of the deep hypersaline anoxic brine lake of the L'Atalante Basin, Eastern Mediterranean Sea (14° C.). All samples were used to produce enrichments cultures to obtain higher biomass levels for the subsequent analyses. The Vulcano Island sediment was introduced into an acidic (pH 1.7) ferrous iron- and sulfate-rich liquid medium and incubated for 4 weeks at 45° C. to produce enrichment VOL; the Koluev Island coastal water was enriched with crude oil and incubated for 4 weeks at 4° C. to produce enrichment KOL; and the seawater:brine interface sample from the L'Atalante Basin was supplemented with glucose and yeast extract and incubated anaerobically for 6 months at 14° C., to produce enrichment L'A. Thus: the microbial communities obtained for analysis represent communities from very distinct, rather extreme habitats: a low energy, low nutrient, heavy metal-rich habitat (VOL), a nutrient and energy-rich, organic pollutant-contaminated habitat (KOL), and a hypersaline, anaerobic environment, inoculated with a sample taken also from a high pressure habitat but which was subsequently maintained at atmospheric pressure, so should contain facultative barophiles (L'A).

### Example 19

General Pan-Reactome Considerations.

As shown in FIG. 6, the VUL reactome consisted of 807 compounds, the KOL reactome consisted of 1493 compounds, and the L'A reactome 2386. The restricted metabolic activity of the VUL sample was not unexpected, since the diversity of the community is low, the biomass concentration is low, and the prevailing physico-chemical conditions highly selective of a restricted metabolism. Similarly, the reactome of KOL was also not unexpected, since the excess carbon in the hydrocarbon-based enrichment leads to high cell densities and much recycling of cellular carbon in all its diverse forms. At first sight, it might seem that the extremely diverse metabolic profile of the L'A metagenome library is surprising, given the highly restricted diversity of the original community. However, it has been shown that a wide range of physico-chemical conditions prevail in the extremely steep chemoclines of the seawater:hypersaline brine interfaces of the brine lakes and this might select for organisms expressing a broader range of metabolic activities. In addition, it should be kept in mind that, on one hand, the oligotrophic environment selects for organisms expressing a wide range of nutrient scavenging systems constitutively at low levels, and in addition, anaerobic metabolism and salt and pressure tolerance systems not specified or expressed by the other two communities, and, on the other, the E. coli cellular environment may result in expression of a range of aerobic metabolism systems encoded, but not necessarily expressed under natural conditions, by the community organisms. Nevertheless, the exceptional richness of the metabolic profile of the L'A library is impressive.

### Example 20

The Micro-Array Served as Experimental Platform to Identify Gene Functions.

The methodology presented here provides a new window to study the functional composition of single cells and microbial communities without apparent need of sequence information as well as to identify many uncharacterized gene-coding enzymes. This has been shown by combining the array concept with high-throughput mass spectrometry peptide sequencing using metabolite-containing nanoparticles. To further prove this hypothesis we extent this analysis not only for P. putida extracts but also for the metagenome-derived extracts to analyse the possibility of mining protein diversity.

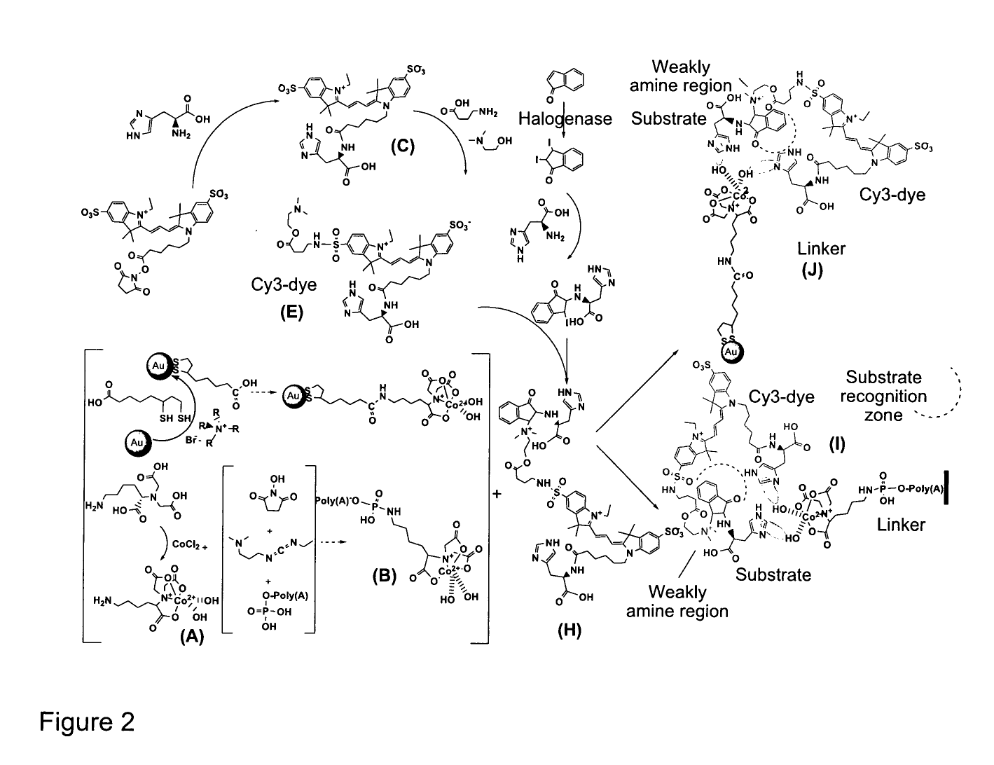

To test this, we randomly select nine SMs exhibiting positive signals (fluorescence values up to 35512) in the micro-array: four for P. putida lysates (cis-2-hydroxypenta-2,4-dienoate, γ-carboxymuconolactone, 3-hydroxyanthranilate and dimethylallyl diphosphate) and five for KOL, VUL and L'A lysates (undecane, (S)-4,5-dihydroxypentan-2,3-dione, 2-bromo-1-chloropropane, phosphatidylinositol-4,5-bisphosphate and methyl viologen). To identify the proteins responsible for their transformation the corresponding Cy3 derivatives were synthesized, immobilized in a gold-magnetic nano-particle, and further incubated with P. putida or library lysates (FIG. 2, right). After 30 min at 20° C. incubation, the gold particles were separated by either magnetic attachment or centrifugation (5000 g×15 min) and the attached proteins were identified by trypsin digestion followed by Q-TOF sequencing. Using peptide sequence fragments, degenerated oligonucleotides were designed, the corresponding genes were amplified using (meta)genomic DNA, cloned into the pET30 Ek/LIC expression vector and the proteins purified with a 6×His tag. Analysis of fluorescence emission when different amounts of pure protein were incubated with different concentration of substrates and vice versa revealed that the obtained results matched the reactome data (Table 3): Cy3 fluorescence emission increased when increasing the amount of both protein and substrate while inactive proteins do not (FIG. 7).

P. putida proteins: Sequence analysis revealed the identity of proteins acting against cis-2-hydroxypenta-2,4-dienoate, γ-carboxymuconolactone, 3-hydroxyanthranilate and dimethylallyl diphosphate —all of them correspond to the hypothetical proteins with no assigned function annotated PP_{—}1394, PP_{—}1752, PP_{—}2949 and PP_{—}1642 (Table 4). However, the experimental analyses provided here suggest that these enzymes are accordingly new 2-keto-4-pentenoate hydratase, 3-carboxy-cis,cis-muconate cycloisomerase, 3-hydroxyanthranilate 3,4-dioxygenase and isopentenyl-diphosphate delta-isomerase (see Table 4). This has also been shown by examining the activity of the pure proteins against standard substrates (data not shown). The correlation of this identity with genomic context is as follows: here we observed that surrounding compounds are also metabolized by P. putida lysates, thus suggesting the presence of new metabolic pathways. A case of interest is the ability of protein PP_{—}1394 to transform the conversion of cis-2-hydroxypenta-2,4-dienoate to cis-2-hydroxypenta-2,4-dienoate as a part of the biphenyl degradation pathway. This pathway is not annotated in KEGG for P. putida KT2440 strain (cf. above), in spite of the fact that strains F1, GB1 and W619 clearly possess some genes responsible for p-cymene and 4-chlorobiphenyl degradation (see http://www.genome.jp/kegg/). In contrast, array hits were found for the entire set of metabolites ranging from p-cymene to 2-hydroxy-6-oxo-7-methylocta-2,4-dienoate as well as from 4-chlorobiphenyl to cis-2,3-dihydro-2,3-dihydroxy-4′-chlorobiphenyl (fluorescence values up to 6931) (FIG. 7) and further analysis of nanoparticle proteome analysis have also revealed the identity of proteins doing those transformations. These data suggest that the lysate of KT2440 contains enzymes that are able to metabolize those intermediates, even though genome information per se does not provide any evidence for that. This suggests in turn that many proteins may potentially enable, yet unknown, important catabolic activities expanding our knowledge on microbial metabolism of this bacterium.

Metagenomic Proteins:

Sequence analysis revealed that all of metagenomic proteins acting against undecane, (S)-4,5-dihydroxypentan-2,3-dione, 2-bromo-1-chloropropane, phosphatidylinositol-4,5-bisphosphate and methyl viologen exhibited a high degree of similarity to predicted hypothetical proteins with no function assigned (Table 4), except L'A62, a 162-amino acid polypeptide with a predicted molecular mass of 18,068 Da along with estimated pI of 4.55, that exhibited high similarity to a predicted [NiFe] hydrogenase from Carboxythermus hydrogenoformans (7e⁻⁵⁴). Data suggest these enzymes are new alkane hydroxylase, S-ribosylhomocysteine lyase, haloalkane dehalogenase, phosphatidylinositol-bisphosphatase and hydrogenase. To prove that assigned function are correct we select and preliminary characterized one of the most promising enzyme candidates, the L'A62 protein and reaffirmed this was indeed the case. A FTIR (Fourier Transform Infrared) spectrum of protein 62 was recorded at a final concentration of 8 M (FIG. 7). The bands that appear in the 2150-1900 cm⁻¹ region are typical of the 1 carbonyl and 2 cyanide ligands of the active site of standard NiFe-hydrogenases (J. C. Fontecilla-Camps et al., Chem Rev 107, 4273 (2007)). The band of frequency value of 1947 cm⁻¹ and the group of bands between 2080 and 2095 cm⁻¹ correspond to the carbonyl ligand and the cyanide ligands respectively of this type of hydrogenases in the “unready” and “ready” oxidized states (A. De Lacey et al., Biochem Biophys Acta 832, 69 (1985)). The band at 1936 cm⁻¹ can be assigned to the carbonyl ligand of irreversibly inactivated enzyme, whereas the bands at 2060 and 2073 cm⁻¹ can be assigned to the cyanide ligands of the same state. Further, the H₂-uptake activity of protein 62 was measured in a spectrophotometer using methyl viologen as electron acceptor (FIG. 7, inset). The protein isolated under aerobic conditions had the typical lag-phase of several minutes in the activity profile of standard NiFe-hydrogenases in the “unready” oxidized state (V. M. Fernandez et al., Biochim Biophys Acta 832, 69 (1985)). Incubation under H₂ of the protein during several hours led to disappearing of the lag-phase and an increase of the maximum activity. This activation process is also a functional characteristic signature of standard NiFe-hydrogenases. Remarkably, the structural and functional data measured suggest an active site very similar to that of standard NiFe-hydrogenases, although the overall protein structure is unique up to date for this type enzymes (just 1 subunit of very small size, an amino acid sequence with no motifs for iron-sulfur clusters nor with the typical L1 or L2 signatures) (P. M. Vignais, B. Billoud, Chem Rev 107, 4206 (2007)).

Lengthy table referenced here
US20120231972A1-20120913-T00001
Please refer to the end of the specification for access instructions.

Lengthy table referenced here
US20120231972A1-20120913-T00002
Please refer to the end of the specification for access instructions.

Lengthy table referenced here
US20120231972A1-20120913-T00003
Please refer to the end of the specification for access instructions.

Lengthy table referenced here
US20120231972A1-20120913-T00004
Please refer to the end of the specification for access instructions.

LENGTHY TABLES
The patent application contains a lengthy table section. A copy of the table is available in electronic form from the USPTO web site (http://seqdata.uspto.gov/?pageRequest=docDetail&DocID=US20120231972A1). An electronic copy of the table will also be available from the USPTO upon request and payment of the fee set forth in 37 CFR 1.19(b)(3).

1. A probe compound for detecting specific enzyme-substrate interactions comprising a transition metal complex and a reactive component of general formula (X):
His-L_{His-TC}-TC-L_{TC-IC}-IC-L_{IC-His}-His formula (X)
wherein His represents a histidine residue, TC represents a test component, IC represents an indicator component, and each of L_{His-TC}, L_{TC-IC} and L_{IC-His} independently represents optional linker components, wherein the reactive component is linked to the transition metal complex by the two histidine residues.

2. The probe compound according to claim 1, wherein the transition metal complex comprises a cobalt or copper atom.

3. The probe compound according to claim 1, wherein the transition metal complex further comprises a multidentate ligand.

4. The probe compound according to claim 1, wherein the transition metal complex comprises a nitrotriacetic acid cobalt(II) moiety.

5. The probe compound according to claim 3, wherein the multidentate ligand of the transition metal complex further comprises an anchoring component.

6. The probe compound according to claim 1, wherein the transition metal complex is a cobalt(II) complex of N_{α},N_{α}-bis-(carboxymethyl)-L-lysine.

7. The probe compound according to claim 1, wherein the indicator component comprises a dye.

8. The probe compound according to claim 1, wherein the test component comprises a substrate selected from the group listed in Table 1.

9. The probe compound according to claim 1, wherein the indicator component comprises a fluorescence dye and wherein the linker component L_{TC-IC} comprises a quaternary ammine function.

10. A method of preparing a probe compound according to claim 1, which comprises the steps of:
(a) preparing a transition metal complex by reacting a salt of a transition metal with a ligand molecule;
(b) preparing a reactive component by
(i) linking a test component to a first histidine residue, optionally using a first linker component,
(ii) linking a dye component to a second histidine component, optionally using a second linker component, and
(iii) linking the test component to the dye component, optionally using a third linker component; and
(c) linking the reactive component to the transition metal complex using the first and second histidine residues.

11. An array for detecting enzymes comprising a plurality of different probe compounds according to claim 1.

12. A method for producing an array according to claim 11, comprising
(a) linking an anchoring component to the ligand of the transition metal complex of the probe compound, and
(b) arranging the different probe compounds in an array.

13. An isolation means comprising a nanoparticle and a probe compound according to claim 1.

14. A method for producing an isolation means according to claim 13, comprising
(a) linking an anchoring component to the ligand of the transition metal complex of the probe compound, and
(b) attaching the probe compound to a nanoparticle.

15-16. (canceled)

17. A method for detecting enzymes comprising contacting an analyte solution containing enzymes with the probe compound of claim 1 so that enzymes of the analyte solution are detected.

18. A method for detecting enzymes comprising contacting an analyte solution containing enzymes with the array of claim 11 so that enzymes of the analyte solution are detected.

19. A method for isolating enzymes comprising contacting a sample with the isolation means of claim 13 thereby isolating enzymes of the sample.
